SEQUENCE LISTING (51 sequences)

SEQ ID NO: 1 - protein, 679 aa, synthetic construct
TLQLDDEYRLYSPQVKPDQDIQSWLEQFPQAWAETAGMGLAKQVPPQVIQLKASATPVSVRQYPLSREAREGIWPHVQRLIQQGILVPVQSPWNTPLLPVRKPGTNDYRPVQDLREVNKRVQDIHPTVPNPYNLLSALPPERNWYTVLDLKDAFFCLRLHPTSQPLFAFEWRDPGTGRTGQLTWTRLPQGFKNSPTIFDEALHRDLANFRIQHPQVTLLQYVDDLLLAGATKQDCLEGTKALLLELSDLGYRASAKKAQICRREVTYLGYSLRGGQRWLTEARKKTVVQIPAPTTAKQVREFLGTAGFCRLWIPGFATLAAPLYPLTKEKGEFSWAPEHQKTFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTHTIWASSLPEGTSAQKAELMALTQALRLAEGKSINIYTDSRYAFATAHVHGAIYKQRGLLTSAGREIKNKEEILSLLEALHLPKRLAIIHCPGHQKAKDLISRGNQMADRVAKQAAQAVNLLPIIETPKAPEPRRQ

SEQ ID NO: 2 - protein, 679 aa, synthetic construct
TLQLDDEYRLYSPLVKPDQNIQFWLEQFPQAWAETAGMGLAKQVPPQVIQLKASATPVSVRQYPLSKEAQEGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGTGRTGQLTWTRLPQGFKNSPTIFDEALHRDLANFRIQHPQVTLLQYVDDLLLAGATKQDCLEGTKALLLELSDLGYRASAKKAQICRREVTYLGYSLRDGQRWLTEARKKTVVQIPAPTTAKQVREFLGTAGFCRLWIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTRTIWASSLPEGTSAQKAELMALTQALRLAEGKSINIYTDSRYAFATAHVHGAIYKQRGLLTSAGREIKNKEEILSLLEALHLPKRLAIIHCPGHQKAKDPISRGNQMADRVAKQAAQGVNLLPMIETPKAPEPGRQ

SEQ ID NO: 3 - protein, 676 aa, synthetic construct
TLQLDDEYRLYSPLVKPDQNIQFWLEQFPQAWAETAGMGLAKQVPPQVIQLKASATPVSVRQYPLSKEAREGIRSHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFDEALHRDLANFRIQHPQVTLLQYVDDLLLAGATKQDCLEGTKALLLELSDLGYRASAKKAQICRREVTYLGYSLRGGQRWLMEARKKTVVQIPAPTTAKQVREFLGTAGFCRLWIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEEADEPVTHDCHQLLIEETGVRKDLTDIPLTGEVLTWFTDRSSYVVEGKRMAGAAVVDGTHTIWASSLPEGTSAQKAELMALTQALRLAEGKSINIYTDSRYAFATAHVHRAIYKQRGLLTSAGREIKNKEEILSLLEALHLPKRLAIIHCPGHQKAKDPISRGNQMADRVTKQAAQGVNLLPIIETPKAPEP

SEQ ID NO: 4 - protein, 676 aa, synthetic construct
TLQLDDEYRLYSPQVKPDQDIQSWLEQFPQAWAETAGMGLAKQVPPQVIQLKASATPVSVRQYPLSREAREGIWPHVQRLIQQGILVPVQSPWNTPLLPVRKPGTNDYRPVQDLREVNKRVQDIHPTVPNPYNLLSALPPEQNWYTVLDLKDAFFCLRLHPTSQPLFAFKWRDPGTGRTGQLTWTRLPQGFKNSPTIFDEALHRDLANFRIQHPQVTLLQYVDDLLLAGATKQDCLEGTKALLLELSDLGYRASAKKAQICRREVTYLGYSLRGGQRWLTEARKKTVVQIPAPTTAKQVREFLGTAGFCRLWIPGFATLAAPLYLLTKEKGEFSWAPEHQKAFDAIKKSLLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNACMTHYQSLLLTERVTFAPPAALNPATLLPEETDEPVTHDCHQLLIEETGVRKDLTDIPLTGEVLTWFTDGSSYVVEGKRMAGAAVVDGTRTIWASSLPEGTSAQKAELMALTQALRLAEGKSINIYTDSRYAFATAHVHGAIYKQRGLLTSAGREIKNKEEILSLLEALHLPKRLAIIHCPGHQKAKDLISRGNQMADRVAKQAAQAVNLLPIIETPKAPEP

SEQ ID NO: 5 - protein, 676 aa, synthetic construct
TLQLDDEYRLYSPLVKPDQNIQFWLEQFPQAWAETAGVGLAKQVPPQVIQLKASATPVSVRQYPLSKEAREGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFDEALHRDLANFRIQHPQVTLLQYVDDLLLAGATKQDCLEGTKALLLELSDLGYRASAKKAQICRREVTYLGYSLRGGQRWLTEARKRTVVQIPAPTTAKQVREFLGTAGFCRLWIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTRTIWASSLPEGTSAQKAELMALTQALRLAEGKSINIYTDSRYAFATAHVHGAIYKQRGLLTSAGREIKNKEEILSLLEALHLPKRLAIIHCPGHQKAKDPISRGNQMADRVAKQAAQGVNLLPIIETPKAPEP

SEQ ID NO: 6 - protein, 513 aa, synthetic construct
TLQLDDEYRLYSPLVKPDQNIQFWLEQFPQAWAETAGMGLAKQVPPQVIQLKASATPVSVRQYPLSKEAQEGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGTGRTGQLTWTRLPQGFKNSPTIFDEALHRDLANFRIQHPQVTLLQYVDDLLLAGATKQDCLEGTKALLLELSDLGYRASAKKAQICRREVTYLGYSLRDGQRWLTEARKKTVVQIPAPTTAKQVREFLGTAGFCRLWIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPL

SEQ ID NO: 7 - protein, 513 aa, synthetic construct
TLQLDDEYRLYSPLVKPDQNIQFWLEQFPQAWAETAGMGLAKQVPPQVIQLKASATPVSVRQYPLSKEAQEGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGTGRTGQLTWTRLPQGFKNSPTIFNEALHRDLANFRIQHPQVTLLQYVDDLLLAGATKQDCLEGTKALLLELSDLGYRASAKKAQICRREVTYLGYSLRDGQRWLTEARKKTVVQIPAPTTAKQVREFLGKAGFCRLFIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPL

SEQ ID NO: 8 - protein, 513 aa, synthetic construct
TLQLDDEYRLYSPLVKPDQNIQFWLEQFPQAWAETAGMGLAKQVPPQVIQLKASATPVSVRQYPLSKEAQEGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGTGRTGQLTWTRLPQGFKNSPTIFNEALHRDLANFRIQHPQVTLLQYVDDLLLAGATKQDCLEGTKALLLELSDLGYRASAKKAQICRREVTYLGYSLRDGQRWLTEARKKTVVQIPAPTTAKQVREFLGKAGFCRLFIPGFATLAAPLYPLTKPGTLFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPL

SEQ ID NO: 9 - protein, 513 aa, synthetic construct
TLQLDDEYRLYSPQVKPDQDIQSWLEQFPQAWAETAGMGLAKQVPPQVIQLKASATPVSVRQYPLSREAREGIWPHVQRLIQQGILVPVQSPWNTPLLPVRKPGTNDYRPVQDLREVNKRVQDIHPTVPNPYNLLSALPPERNWYTVLDLKDAFFCLRLHPTSQPLFAFEWRDPGTGRTGQLTWTRLPQGFKNSPTIFDEALHRDLANFRIQHPQVTLLQYVDDLLLAGATKQDCLEGTKALLLELSDLGYRASAKKAQICRREVTYLGYSLRGGQRWLTEARKKTVVQIPAPTTAKQVREFLGTAGFCRLWIPGFATLAAPLYPLTKEKGEFSWAPEHQKTFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPL

SEQ ID NO: 10 - protein, 513 aa, synthetic construct
TLQLDDEYRLYSPQVKPDQDIQSWLEQFPQAWAETAGMGLAKQVPPQVIQLKASATPVSVRQYPLSREAREGIWPHVQRLIQQGILVPVQSPWNTPLLPVRKPGTNDYRPVQDLREVNKRVQDIHPTVPNPYNLLSALPPERNWYTVLDLKDAFFCLRLHPTSQPLFAFEWRDPGTGRTGQLTWTRLPQGFKNSPTIFNEALHRDLANFRIQHPQVTLLQYVDDLLLAGATKQDCLEGTKALLLELSDLGYRASAKKAQICRREVTYLGYSLRGGQRWLTEARKKTVVQIPAPTTAKQVREFLGKAGFCRLFIPGFATLAAPLYPLTKEKGEFSWAPEHQKTFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPL

SEQ ID NO: 11 - protein, 513 aa, synthetic construct
TLQLDDEYRLYSPQVKPDQDIQSWLEQFPQAWAETAGMGLAKQVPPQVIQLKASATPVSVRQYPLSREAREGIWPHVQRLIQQGILVPVQSPWNTPLLPVRKPGTNDYRPVQDLREVNKRVQDIHPTVPNPYNLLSALPPERNWYTVLDLKDAFFCLRLHPTSQPLFAFEWRDPGTGRTGQLTWTRLPQGFKNSPTIFNEALHRDLANFRIQHPQVTLLQYVDDLLLAGATKQDCLEGTKALLLELSDLGYRASAKKAQICRREVTYLGYSLRGGQRWLTEARKKTVVQIPAPTTAKQVREFLGKAGFCRLFIPGFATLAAPLYPLTKPGTLFSWAPEHQKTFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPL

SEQ ID NO: 12 - protein, 682 aa, synthetic construct
TLQLDDEYRLYSPLVKPDQNIQFWLEQFPQAWAETAGMGLAKQVPPQVIQLKASATPVSVRQYPLSKEAREGIRSHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFNEALHRDLANFRIQHPQVTLLQYVDDLLLAGATKQDCLEGTKALLLELSDLGYRASAKKAQICRREVTYLGYSLRGGQRWLMEARKKTVVQIPAPTTAKQVREFLGKAGFCRLFIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEEADEPVTHDCHQLLIEETGVRKDLTDIPLTGEVLTWFTDRSSYVVEGKRMAGAAVVDGTHTIWASSLPEGTSAQKAELMALTQALRLAEGKSINIYTDSRYAFATAHVHRAIYKQRGWLTSAGREIKNKEEILSLLEALHLPKRLAIIHCPGHQKAKDPISRGNQMADRVTKQAAQGVNLLPIIETPKAPEPRNASEH

SEQ ID NO: 13 - protein, 676 aa, synthetic construct
TLQLDDEYRLYSPQVKPDQDIQSWLEQFPQAWAETAGMGLAKQVPPQVIQLKASATPVSVRQYPLSREAREGIWPHVQRLIQQGILVPVQSPWNTPLLPVRKPGTNDYRPVQDLREVNKRVQDIHPTVPNPYNLLSALPPEQNWYTVLDLKDAFFCLRLHPTSQPLFAFKWRDPGTGRTGQLTWTRLPQGFKNSPTIFNEALHRDLANFRIQHPQVTLLQYVDDLLLAGATKQDCLEGTKALLLELSDLGYRASAKKAQICRREVTYLGYSLRGGQRWLTEARKKTVVQIPAPTTAKQVREFLGKAGFCRLFIPGFATLAAPLYLLTKEKGEFSWAPEHQKAFDAIKKSLLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNACMTHYQSLLLTERVTFAPPAALNPATLLPEETDEPVTHDCHQLLIEETGVRKDLTDIPLTGEVLTWFTDGSSYVVEGKRMAGAAVVDGTRTIWASSLPEGTSAQKAELMALTQALRLAEGKSINIYTDSRYAFATAHVHGAIYKQRGWLTSAGREIKNKEEILSLLEALHLPKRLAIIHCPGHQKAKDLISRGNQMADRVAKQAAQAVNLLPIIETPKAPEP

SEQ ID NO: 14 - protein, 676 aa, synthetic construct
TLQLDDEYRLYSPLVKPDQNIQFWLEQFPQAWAETAGVGLAKQVPPQVIQLKASATPVSVRQYPLSKEAREGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFNEALHRDLANFRIQHPQVTLLQYVDDLLLAGATKQDCLEGTKALLLELSDLGYRASAKKAQICRREVTYLGYSLRGGQRWLTEARKRTVVQIPAPTTAKQVREFLGKAGFCRLFIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTRTIWASSLPEGTSAQKAELMALTQALRLAEGKSINIYTDSRYAFATAHVHGAIYKQRGWLTSAGREIKNKEEILSLLEALHLPKRLAIIHCPGHQKAKDPISRGNQMADRVAKQAAQGVNLLPIIETPKAPEP

SEQ ID NO: 15 - protein, 513 aa, synthetic construct
TLQLDDEYRLYSPLVKPDQNIQFWLEQFPQAWAETAGMGLAKQVPPQVIQLKASATPVSVRQYPLSKEAREGIRSHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFNEALHRDLANFRIQHPQVTLLQYVDDLLLAGATKQDCLEGTKALLLELSDLGYRASAKKAQICRREVTYLGYSLRGGQRWLMEARKKTVVQIPAPTTAKQVREFLGKAGFCRLFIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEEADEPVTHDCHQLLIEETGVRKDLTDIPL

SEQ ID NO: 16 - protein, 513 aa, synthetic construct
TLQLDDEYRLYSPQVKPDQDIQSWLEQFPQAWAETAGMGLAKQVPPQVIQLKASATPVSVRQYPLSREAREGIWPHVQRLIQQGILVPVQSPWNTPLLPVRKPGTNDYRPVQDLREVNKRVQDIHPTVPNPYNLLSALPPEQNWYTVLDLKDAFFCLRLHPTSQPLFAFKWRDPGTGRTGQLTWTRLPQGFKNSPTIFNEALHRDLANFRIQHPQVTLLQYVDDLLLAGATKQDCLEGTKALLLELSDLGYRASAKKAQICRREVTYLGYSLRGGQRWLTEARKKTVVQIPAPTTAKQVREFLGKAGFCRLFIPGFATLAAPLYLLTKEKGEFSWAPEHQKAFDAIKKSLLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNACMTHYQSLLLTERVTFAPPAALNPATLLPEETDEPVTHDCHQLLIEETGVRKDLTDIPL

SEQ ID NO: 17 - protein, 513 aa, synthetic construct
TLQLDDEYRLYSPLVKPDQNIQFWLEQFPQAWAETAGVGLAKQVPPQVIQLKASATPVSVRQYPLSKEAREGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFNEALHRDLANFRIQHPQVTLLQYVDDLLLAGATKQDCLEGTKALLLELSDLGYRASAKKAQICRREVTYLGYSLRGGQRWLTEARKRTVVQIPAPTTAKQVREFLGKAGFCRLFIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPL

SEQ ID NO: 18 - protein, 1386 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 19 - protein, 56 aa, synthetic construct
DFRKIRSGPRQSGNLGNRTPLDKDQCAYCKEKGHWARNCPKKGNKGLKVLALEEDK

SEQ ID NO: 20 - protein, 33 aa, synthetic construct
SGGSSGGSSGSETPGTSESATPESSGGSSGGSS

SEQ ID NO: 21 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 22 - protein, 2025 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSDFRKIRSGPRQSGNLGNRTPLDKDQCAYCKEKGHWARNCPKKGNKGLKVLALEEDKSGSETPGTSESATPESTLQLDDEYRLYSPLVKPDQNIQFWLEQFPQAWAETAGMGLAKQVPPQVIQLKASATPVSVRQYPLSKEAQEGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGTGRTGQLTWTRLPQGFKNSPTIFNEALHRDLANFRIQHPQVTLLQYVDDLLLAGATKQDCLEGTKALLLELSDLGYRASAKKAQICRREVTYLGYSLRDGQRWLTEARKKTVVQIPAPTTAKQVREFLGKAGFCRLFIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLSGGSKRTADGSEFEPKKKRKV

SEQ ID NO: 23 - protein, 2025 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSDFRKIRSGPRQSGNLGNRTPLDKDQCAYCKEKGHWARNCPKKGNKGLKVLALEEDKSGSETPGTSESATPESTLQLDDEYRLYSPLVKPDQNIQFWLEQFPQAWAETAGMGLAKQVPPQVIQLKASATPVSVRQYPLSKEAQEGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGTGRTGQLTWTRLPQGFKNSPTIFNEALHRDLANFRIQHPQVTLLQYVDDLLLAGATKQDCLEGTKALLLELSDLGYRASAKKAQICRREVTYLGYSLRDGQRWLTEARKKTVVQIPAPTTAKQVREFLGKAGFCRLFIPGFATLAAPLYPLTKPGTLFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLSGGSKRTADGSEFEPKKKRKV

SEQ ID NO: 24 - protein, 2025 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSDFRKIRSGPRQSGNLGNRTPLDKDQCAYCKEKGHWARNCPKKGNKGPKVLALEEDKSGSETPGTSESATPESTLQLDDEYRLYSPQVKPDQDIQSWLEQFPQAWAETAGMGLAKQVPPQVIQLKASATPVSVRQYPLSREAREGIWPHVQRLIQQGILVPVQSPWNTPLLPVRKPGTNDYRPVQDLREVNKRVQDIHPTVPNPYNLLSALPPERNWYTVLDLKDAFFCLRLHPTSQPLFAFEWRDPGTGRTGQLTWTRLPQGFKNSPTIFNEALHRDLANFRIQHPQVTLLQYVDDLLLAGATKQDCLEGTKALLLELSDLGYRASAKKAQICRREVTYLGYSLRGGQRWLTEARKKTVVQIPAPTTAKQVREFLGKAGFCRLFIPGFATLAAPLYPLTKEKGEFSWAPEHQKTFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLSGGSKRTADGSEFEPKKKRKV

SEQ ID NO: 25 - protein, 2025 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSDFRKIRSGPRQSGNLGNRTPLDKDQCAYCKEKGHWARNCPKKGNKGPKVLALEEDKSGSETPGTSESATPESTLQLDDEYRLYSPQVKPDQDIQSWLEQFPQAWAETAGMGLAKQVPPQVIQLKASATPVSVRQYPLSREAREGIWPHVQRLIQQGILVPVQSPWNTPLLPVRKPGTNDYRPVQDLREVNKRVQDIHPTVPNPYNLLSALPPERNWYTVLDLKDAFFCLRLHPTSQPLFAFEWRDPGTGRTGQLTWTRLPQGFKNSPTIFNEALHRDLANFRIQHPQVTLLQYVDDLLLAGATKQDCLEGTKALLLELSDLGYRASAKKAQICRREVTYLGYSLRGGQRWLTEARKKTVVQIPAPTTAKQVREFLGKAGFCRLFIPGFATLAAPLYPLTKPGTLFSWAPEHQKTFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLSGGSKRTADGSEFEPKKKRKV

SEQ ID NO: 26 - protein, 2116 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLQLDDEYRLYSPLVKPDQNIQFWLEQFPQAWAETAGMGLAKQVPPQVIQLKASATPVSVRQYPLSKEAREGIRSHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFDEALHRDLANFRIQHPQVTLLQYVDDLLLAGATKQDCLEGTKALLLELSDLGYRASAKKAQICRREVTYLGYSLRGGQRWLMEARKKTVVQIPAPTTAKQVREFLGTAGFCRLWIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEEADEPVTHDCHQLLIEETGVRKDLTDIPLTGEVLTWFTDRSSYVVEGKRMAGAAVVDGTHTIWASSLPEGTSAQKAELMALTQALRLAEGKSINIYTDSRYAFATAHVHRAIYKQRGLLTSAGREIKNKEEILSLLEALHLPKRLAIIHCPGHQKAKDPISRGNQMADRVTKQAAQGVNLLPIIETPKAPEPSGGSKRTADGSEFEPKKKRKV

SEQ ID NO: 27 - protein, 2116 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLQLDDEYRLYSPQVKPDQDIQSWLEQFPQAWAETAGMGLAKQVPPQVIQLKASATPVSVRQYPLSREAREGIWPHVQRLIQQGILVPVQSPWNTPLLPVRKPGTNDYRPVQDLREVNKRVQDIHPTVPNPYNLLSALPPEQNWYTVLDLKDAFFCLRLHPTSQPLFAFKWRDPGTGRTGQLTWTRLPQGFKNSPTIFDEALHRDLANFRIQHPQVTLLQYVDDLLLAGATKQDCLEGTKALLLELSDLGYRASAKKAQICRREVTYLGYSLRGGQRWLTEARKKTVVQIPAPTTAKQVREFLGTAGFCRLWIPGFATLAAPLYLLTKEKGEFSWAPEHQKAFDAIKKSLLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNACMTHYQSLLLTERVTFAPPAALNPATLLPEETDEPVTHDCHQLLIEETGVRKDLTDIPLTGEVLTWFTDGSSYVVEGKRMAGAAVVDGTRTIWASSLPEGTSAQKAELMALTQALRLAEGKSINIYTDSRYAFATAHVHGAIYKQRGLLTSAGREIKNKEEILSLLEALHLPKRLAIIHCPGHQKAKDLISRGNQMADRVAKQAAQAVNLLPIIETPKAPEPSGGSKRTADGSEFEPKKKRKV

SEQ ID NO: 28 - protein, 2116 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLQLDDEYRLYSPLVKPDQNIQFWLEQFPQAWAETAGVGLAKQVPPQVIQLKASATPVSVRQYPLSKEAREGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFDEALHRDLANFRIQHPQVTLLQYVDDLLLAGATKQDCLEGTKALLLELSDLGYRASAKKAQICRREVTYLGYSLRGGQRWLTEARKRTVVQIPAPTTAKQVREFLGTAGFCRLWIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTRTIWASSLPEGTSAQKAELMALTQALRLAEGKSINIYTDSRYAFATAHVHGAIYKQRGLLTSAGREIKNKEEILSLLEALHLPKRLAIIHCPGHQKAKDPISRGNQMADRVAKQAAQGVNLLPIIETPKAPEPSGGSKRTADGSEFEPKKKRKV

SEQ ID NO: 29 - protein, 2116 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLQLDDEYRLYSPLVKPDQNIQFWLEQFPQAWAETAGMGLAKQVPPQVIQLKASATPVSVRQYPLSKEAREGIRSHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFNEALHRDLANFRIQHPQVTLLQYVDDLLLAGATKQDCLEGTKALLLELSDLGYRASAKKAQICRREVTYLGYSLRGGQRWLMEARKKTVVQIPAPTTAKQVREFLGKAGFCRLFIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEEADEPVTHDCHQLLIEETGVRKDLTDIPLTGEVLTWFTDRSSYVVEGKRMAGAAVVDGTHTIWASSLPEGTSAQKAELMALTQALRLAEGKSINIYTDSRYAFATAHVHRAIYKQRGWLTSAGREIKNKEEILSLLEALHLPKRLAIIHCPGHQKAKDPISRGNQMADRVTKQAAQGVNLLPIIETPKAPEPSGGSKRTADGSEFEPKKKRKV

SEQ ID NO: 30 - protein, 2116 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLQLDDEYRLYSPQVKPDQDIQSWLEQFPQAWAETAGMGLAKQVPPQVIQLKASATPVSVRQYPLSREAREGIWPHVQRLIQQGILVPVQSPWNTPLLPVRKPGTNDYRPVQDLREVNKRVQDIHPTVPNPYNLLSALPPEQNWYTVLDLKDAFFCLRLHPTSQPLFAFKWRDPGTGRTGQLTWTRLPQGFKNSPTIFNEALHRDLANFRIQHPQVTLLQYVDDLLLAGATKQDCLEGTKALLLELSDLGYRASAKKAQICRREVTYLGYSLRGGQRWLTEARKKTVVQIPAPTTAKQVREFLGKAGFCRLFIPGFATLAAPLYLLTKEKGEFSWAPEHQKAFDAIKKSLLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNACMTHYQSLLLTERVTFAPPAALNPATLLPEETDEPVTHDCHQLLIEETGVRKDLTDIPLTGEVLTWFTDGSSYVVEGKRMAGAAVVDGTRTIWASSLPEGTSAQKAELMALTQALRLAEGKSINIYTDSRYAFATAHVHGAIYKQRGWLTSAGREIKNKEEILSLLEALHLPKRLAIIHCPGHQKAKDLISRGNQMADRVAKQAAQAVNLLPIIETPKAPEPSGGSKRTADGSEFEPKKKRKV

SEQ ID NO: 31 - protein, 2116 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLQLDDEYRLYSPLVKPDQNIQFWLEQFPQAWAETAGVGLAKQVPPQVIQLKASATPVSVRQYPLSKEAREGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFNEALHRDLANFRIQHPQVTLLQYVDDLLLAGATKQDCLEGTKALLLELSDLGYRASAKKAQICRREVTYLGYSLRGGQRWLTEARKRTVVQIPAPTTAKQVREFLGKAGFCRLFIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTRTIWASSLPEGTSAQKAELMALTQALRLAEGKSINIYTDSRYAFATAHVHGAIYKQRGWLTSAGREIKNKEEILSLLEALHLPKRLAIIHCPGHQKAKDPISRGNQMADRVAKQAAQGVNLLPIIETPKAPEPSGGSKRTADGSEFEPKKKRKV

SEQ ID NO: 32 - protein, 2188 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSDFRKIRSGPRQSGNLGNRTPLDKDQCAYCKEKGHWARDCPKKGNKGLKVLALEEDKSGSETPGTSESATPESTLQLDDEYRLYSPLVKPDQNIQFWLEQFPQAWAETAGMGLAKQVPPQVIQLKASATPVSVRQYPLSKEAREGIRSHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFNEALHRDLANFRIQHPQVTLLQYVDDLLLAGATKQDCLEGTKALLLELSDLGYRASAKKAQICRREVTYLGYSLRGGQRWLMEARKKTVVQIPAPTTAKQVREFLGKAGFCRLFIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEEADEPVTHDCHQLLIEETGVRKDLTDIPLTGEVLTWFTDRSSYVVEGKRMAGAAVVDGTHTIWASSLPEGTSAQKAELMALTQALRLAEGKSINIYTDSRYAFATAHVHRAIYKQRGWLTSAGREIKNKEEILSLLEALHLPKRLAIIHCPGHQKAKDPISRGNQMADRVTKQAAQGVNLLPIIETPKAPEPSGGSKRTADGSEFEPKKKRKV

SEQ ID NO: 33 - protein, 2188 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSDFRKIRSGPRQSGNLGNRTPLDKDQCAYCKKKGHWARNCPKKGNKGPKVLALEEDKSGSETPGTSESATPESTLQLDDEYRLYSPQVKPDQDIQSWLEQFPQAWAETAGMGLAKQVPPQVIQLKASATPVSVRQYPLSREAREGIWPHVQRLIQQGILVPVQSPWNTPLLPVRKPGTNDYRPVQDLREVNKRVQDIHPTVPNPYNLLSALPPEQNWYTVLDLKDAFFCLRLHPTSQPLFAFKWRDPGTGRTGQLTWTRLPQGFKNSPTIFNEALHRDLANFRIQHPQVTLLQYVDDLLLAGATKQDCLEGTKALLLELSDLGYRASAKKAQICRREVTYLGYSLRGGQRWLTEARKKTVVQIPAPTTAKQVREFLGKAGFCRLFIPGFATLAAPLYLLTKEKGEFSWAPEHQKAFDAIKKSLLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNACMTHYQSLLLTERVTFAPPAALNPATLLPEETDEPVTHDCHQLLIEETGVRKDLTDIPLTGEVLTWFTDGSSYVVEGKRMAGAAVVDGTRTIWASSLPEGTSAQKAELMALTQALRLAEGKSINIYTDSRYAFATAHVHGAIYKQRGWLTSAGREIKNKEEILSLLEALHLPKRLAIIHCPGHQKAKDLISRGNQMADRVAKQAAQAVNLLPIIETPKAPEPSGGSKRTADGSEFEPKKKRKV

SEQ ID NO: 34 - protein, 2188 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSDFRKIRSGPRQLGNLGNRTPLDKDQCAYCKEKGHWARDCPKKGNKGLKVLALEEDKSGSETPGTSESATPESTLQLDDEYRLYSPLVKPDQNIQFWLEQFPQAWAETAGVGLAKQVPPQVIQLKASATPVSVRQYPLSKEAREGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFNEALHRDLANFRIQHPQVTLLQYVDDLLLAGATKQDCLEGTKALLLELSDLGYRASAKKAQICRREVTYLGYSLRGGQRWLTEARKRTVVQIPAPTTAKQVREFLGKAGFCRLFIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTRTIWASSLPEGTSAQKAELMALTQALRLAEGKSINIYTDSRYAFATAHVHGAIYKQRGWLTSAGREIKNKEEILSLLEALHLPKRLAIIHCPGHQKAKDPISRGNQMADRVAKQAAQGVNLLPIIETPKAPEPSGGSKRTADGSEFEPKKKRKV

SEQ ID NO: 35 - protein, 199 aa, synthetic construct
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 36 - protein, 56 aa, synthetic construct
DFRKIRSGPRQSGNLGNRTPLDKDQCAYCKEKGHWARNCPKKGNKGPKVLALEEDK

SEQ ID NO: 37 - protein, 56 aa, synthetic construct
DFRKIRSGPRQSGNLGNRTPLDKDQCAYCKEKGHWARDCPKKGNKGLKVLALEEDK

SEQ ID NO: 38 - protein, 56 aa, synthetic construct
DFRKIRSGPRQSGNLGNRTPLDKDQCAYCKKKGHWARNCPKKGNKGPKVLALEEDK

SEQ ID NO: 39 - protein, 56 aa, synthetic construct
DFRKIRSGPRQLGNLGNRTPLDKDQCAYCKEKGHWARDCPKKGNKGLKVLALEEDK

SEQ ID NO: 40 - protein, 659 aa, synthetic construct
TLQLDDEYRLYSPLVKPDQNIQFWLEQFPQAWAETAGVGLAKQVPPQVIQLKASATPVSVRQYPLSKEAREGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFNEALHRDLANFRIQHPQVTLLQYADDLLLAGATKQDCLEGTKALLLELSDLGYRASAKKAQICRREVTYLGYSLRGGQRWLTEARKRTVVQIPAPTTAKQVREFLGKAGFCRLFIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTRTIWASSLPEGTSAQKAELMALTQALRLAEGKSINIYTDSRYAFATAHVHGAIYKQRGWLTSAGREIKNKEEILSLLEALHLPKRLAIIHCPGHQKAKDPISRGNQMADRVAKQAA

SEQ ID NO: 41 - protein, 659 aa, synthetic construct
TLQLDDEYRLYSPLVKPDQNIQFWLEQFPQAWAETAGVGLAKQVPPQVIQLKASATPVSVRQRPLSKEAREGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFNEALHRDLANFRIQHPQVTLLQYADDLLLAGATKQDCLEGTKALLLELSDLGYRASAKKAQICRREVTYLGYSLRGGQRWLTEARKRTVVQIPAPTTAKQVREFLGKAGFCRLFIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTRTIWASSLPEGTSAQKAELMALTQALRLAEGKSINIYTDSRYAFATAHVHGAIYKQRGWLTSAGREIKNKEEILSLLEALHLPKRLAIIHCPGHQKAKDPISRGNQMADRVAKQAA

SEQ ID NO: 42 - protein, 659 aa, synthetic construct
TLQLDDEYRLYSPLVKPDQNIQFWLEQFPQAWAETAGVGLAKQVPPQVIQLKASATPVSVRQYPLSKEAREGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFNEALHRDLANFRIQHPQVTLLQYADDLLLAGATKQDCLEGTKALLLELSDLGYRASAKKAQICRREVTYLGYSLRGGQRWLTEARKRTVVQIPAPTTAKQVREFLGKAGFCRLFIPGFATLAAPLYPLTKEKGEFSWAPEHQKAFDAIKKALLSAPALALPDVTKPFTLYVDERKGVARGVLTQTLGPWRRPVAYLSKKLDPVASGWPVRLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTRTIWASSLPEGTSAQKAELMALTQALRLAEGKSINIYTDSRYAFATAHVHGAIYKQRGWLTSAGREIKNKEEILSLLEALHLPKRLAIIHCPGHQKAKDPISRGNQMADRVAKQAA

SEQ ID NO: 43 - protein, 659 aa, synthetic construct
TLQLDDEYRLYSPLVKPDQNIQFWLEQFPQAWAETAGVGLAKQVPPQVIQLKASATPVSVRQRPLSKEAREGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFNEALHRDLANFRIQHPQVTLLQYADDLLLAGATKQDCLEGTKALLLELSDLGYRASAKKAQICRREVTYLGYSLRGGQRWLTEARKRTVVQIPAPTTAKQVREFLGKAGFCRLFIPGFATLAAPLYPLTKEKGEFSWAPEHQKAFDAIKKALLSAPALALPDVTKPFTLYVDERKGVARGVLTQTLGPWRRPVAYLSKKLDPVASGWPVRLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTRTIWASSLPEGTSAQKAELMALTQALRLAEGKSINIYTDSRYAFATAHVHGAIYKQRGWLTSAGREIKNKEEILSLLEALHLPKRLAIIHCPGHQKAKDPISRGNQMADRVAKQAA

SEQ ID NO: 44 - protein, 513 aa, synthetic construct
TLQLDDEYRLYSPLVKPDQNIQFWLEQFPQAWAETAGVGLAKQVPPQVIQLKASATPVSVRQRPLSKEAREGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFNEALHRDLANFRIQHPQVTLLQYADDLLLAGATKQDCLEGTKALLLELSDLGYRASAKKAQICRREVTYLGYSLRGGQRWLTEARKRTVVQIPAPTTAKQVREFLGKAGFCRLFIPGFATLAAPLYPLTKEKGEFSWAPEHQKAFDAIKKALLSAPALALPDVTKPFTLYVDERKGVARGVLTQTLGPWRRPVAYLSKKLDPVASGWPVRLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPL

SEQ ID NO: 45 - protein, 2078 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLQLDDEYRLYSPLVKPDQNIQFWLEQFPQAWAETAGVGLAKQVPPQVIQLKASATPVSVRQYPLSKEAREGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFNEALHRDLANFRIQHPQVTLLQYADDLLLAGATKQDCLEGTKALLLELSDLGYRASAKKAQICRREVTYLGYSLRGGQRWLTEARKRTVVQIPAPTTAKQVREFLGKAGFCRLFIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTRTIWASSLPEGTSAQKAELMALTQALRLAEGKSINIYTDSRYAFATAHVHGAIYKQRGWLTSAGREIKNKEEILSLLEALHLPKRLAIIHCPGHQKAKDPISRGNQMADRVAKQAA

SEQ ID NO: 46 - protein, 2078 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLQLDDEYRLYSPLVKPDQNIQFWLEQFPQAWAETAGVGLAKQVPPQVIQLKASATPVSVRQRPLSKEAREGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFNEALHRDLANFRIQHPQVTLLQYADDLLLAGATKQDCLEGTKALLLELSDLGYRASAKKAQICRREVTYLGYSLRGGQRWLTEARKRTVVQIPAPTTAKQVREFLGKAGFCRLFIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTRTIWASSLPEGTSAQKAELMALTQALRLAEGKSINIYTDSRYAFATAHVHGAIYKQRGWLTSAGREIKNKEEILSLLEALHLPKRLAIIHCPGHQKAKDPISRGNQMADRVAKQAA

SEQ ID NO: 47 - protein, 2078 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLQLDDEYRLYSPLVKPDQNIQFWLEQFPQAWAETAGVGLAKQVPPQVIQLKASATPVSVRQYPLSKEAREGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFNEALHRDLANFRIQHPQVTLLQYADDLLLAGATKQDCLEGTKALLLELSDLGYRASAKKAQICRREVTYLGYSLRGGQRWLTEARKRTVVQIPAPTTAKQVREFLGKAGFCRLFIPGFATLAAPLYPLTKEKGEFSWAPEHQKAFDAIKKALLSAPALALPDVTKPFTLYVDERKGVARGVLTQTLGPWRRPVAYLSKKLDPVASGWPVRLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTRTIWASSLPEGTSAQKAELMALTQALRLAEGKSINIYTDSRYAFATAHVHGAIYKQRGWLTSAGREIKNKEEILSLLEALHLPKRLAIIHCPGHQKAKDPISRGNQMADRVAKQAA

SEQ ID NO: 48 - protein, 2078 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLQLDDEYRLYSPLVKPDQNIQFWLEQFPQAWAETAGVGLAKQVPPQVIQLKASATPVSVRQRPLSKEAREGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFNEALHRDLANFRIQHPQVTLLQYADDLLLAGATKQDCLEGTKALLLELSDLGYRASAKKAQICRREVTYLGYSLRGGQRWLTEARKRTVVQIPAPTTAKQVREFLGKAGFCRLFIPGFATLAAPLYPLTKEKGEFSWAPEHQKAFDAIKKALLSAPALALPDVTKPFTLYVDERKGVARGVLTQTLGPWRRPVAYLSKKLDPVASGWPVRLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTRTIWASSLPEGTSAQKAELMALTQALRLAEGKSINIYTDSRYAFATAHVHGAIYKQRGWLTSAGREIKNKEEILSLLEALHLPKRLAIIHCPGHQKAKDPISRGNQMADRVAKQAA

SEQ ID NO: 49 - protein, 1932 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLQLDDEYRLYSPLVKPDQNIQFWLEQFPQAWAETAGVGLAKQVPPQVIQLKASATPVSVRQRPLSKEAREGIRPHVQRLIQQGILVPVQSPWNTPLLPVRKPGTNDYRPVQDLREVNKRVQDIHPTVPNPYNLLCALPPQRSWYTVLDLKDAFFCLRLHPTSQPLFAFEWRDPGAGRTGQLTWTRLPQGFKNSPTIFNEALHRDLANFRIQHPQVTLLQYADDLLLAGATKQDCLEGTKALLLELSDLGYRASAKKAQICRREVTYLGYSLRGGQRWLTEARKRTVVQIPAPTTAKQVREFLGKAGFCRLFIPGFATLAAPLYPLTKEKGEFSWAPEHQKAFDAIKKALLSAPALALPDVTKPFTLYVDERKGVARGVLTQTLGPWRRPVAYLSKKLDPVASGWPVRLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPL

SEQ ID NO: 50 - RNA, 20 nt, synthetic construct
  source 1..20: mol_type = other RNA
gttcactgccgtataggcag

SEQ ID NO: 51 - RNA, 15 nt, synthetic construct
  source 1..15: mol_type = other RNA
ctgccgtataggcag

(19) United States
(12) Patent Application Publication
(10) Pub. No.: US 2026/0275307 A1
(43) Pub. Date: Sep. 17, 2026

(54) PRIME EDITING SYSTEM BASED ON PERV REVERSE TRANSCRIPTASE

(71) Applicants: Jiangxi Agricultural University, Nanchang, Jiangxi (CN); lnstitute of Genetics and Developmental Biology, Chinese Academy of Sciences, Beijing (CN)

(72) Inventors: Lusheng HUANG, Nanchang, Jiangxi (CN); Yuyun XING, Nanchang, Jiangxi (CN); Weiwei LIU, Nanchang, Jiangxi (CN); Wenxin DUAN, Nanchang, Jiangxi (CN); Caixia GAO, Chaoyang District, Beijing (CN)

(21) Appl. No.: 18/995,978

(22) PCT Filed: May 21, 2024
(86) PCT No.: PCT/CN2024/094408

§ 371 Date: Mar. 31, 2026

(30) Foreign Application Priority Data
May 22, 2023 (CN) 202310578085.0

(51) Int. Cl. C12N 9/12; C12N 9/22; C12N 15/11; C12N 15/90

(52) U.S. Cl. CPC C12N 9/1276; C12N 9/226; C12N 15/11; C12N 15/907; C12Y 207/07049; C12N 2310/20

(57) ABSTRACT

The present invention relates to the field of genetic engineering. Specifically, the present invention relates to a prime editing system based on PERV reverse transcriptase, and a method for gene editing by said system. More specifically, the present invention provides: 1. a prime editing system using a PERV-RT with PERV-NC fusion, RNase H domain deletion and multiple mutations; 2. a pegRNA fused with a truncated Csy4 (tCsy4-pegRNA); 3. one or more small molecule compounds and combinations thereof for treating cells; to improve the efficiency of prime editing in mammals.

### TECHNICAL FIELD

The present invention relates to the field of genetic engineering. Specifically, the present invention relates to a prime editing system based on PERV reverse transcriptase, and a method for gene editing by the system.

### BACKGROUND OF THE INVENTION

Prime Editing (PE) is a new gene editing technology emerging in recent years, which can realize various types of editing such as point mutation, insertion, deletion, and substitution without generating DNA double-strand breaks, overcomes the main shortcomings of the current CRISPR/Cas9 and single-base editing technologies, and is currently the most accurate genome editing technology. The PE editing system consists of a PE editor and a prime editing guide RNA (pegRNA and/or ngRNA). The PE editor is the most important component of prime editing, and the PE editor is formed by fusing a Cas9 single nickase (nCas9 H840A) and a reverse transcriptase (RT). At present, editor PE2 and its modified version PEmax, which are more efficient and widely used, use the reverse transcriptase of murine mooney murine leukemia virus (MMLV-RT) carrying five mutations D200N, L603W, T330P, T306K and W313F.

There are teams using other types of RTs for prime editing, but they are less efficient or significantly less efficient than PE2. In addition, it is reported that the fusion of the nucleocapsid protein (NC) to RT helps to improve the prime editing efficiency, and an efficiency-enhancing PE editor ePPE has been developed by fusing the nucleocapsid protein (NC) of MMLV to MMLV-RT with the RNase H structure deleted (MMLV-RT (ΔRNase H)). However, reports to date have shown that ePPE only significantly improves editing efficiency in plant cells and does not significantly improve editing efficiency in mammalian cells.

There remains a need in the art for efficient prime editing systems, particularly those capable of efficient editing in mammalian cells.

### SUMMARY OF THE INVENTION

The present invention uses a PERV-NC fused PERV-RT having the RNase H structure deleted and carrying multiple mutation sites to replace MMLV-RT in PE2 editor to improve the efficiency of prime editing in mammals.

### BRIEF DESCRIPTION OF THE DRAWINGS

FIG. 1 shows the amino acid sequence alignment of MMLV-RT (ΔRNase H), PERV-AC-RT (ΔRNase H) and PERV-B-RT (ΔRNase H).

FIG. 2 is a schematic diagram showing vectors of the PERV reverse transcriptase editors of different NCBI sequences of the present invention.

FIG. 3 shows the editing efficiency of PE2c-Puro, epPE-B-M3Δ-Puro, epPE-B-M4Δ*-Puro editors in BFP reporter cell lines.

FIG. 4 shows the editing efficiency of PE2c-Puro, epPE-M3Δ-Puro editors in primary PFF.

FIG. 5 shows the editing efficiency of PE2c-Puro and epPE-AC-M3Δ-Puro editors in HEK293T cells, dog fetal fibroblasts (DFF) and sheep fetal fibroblasts (SFF).

FIG. 6 is a schematic diagram of vectors based on different Bama pig PERV reverse transcriptase editors of the present invention.

FIG. 7 shows the editing activity of the pPE editors containing Bama pig PERV reverse transcriptase with 4 point mutations or wild type Bama pig PERV reverse transcriptase in BFP reporter cell line.

FIG. 8 shows the editing efficiency of pPE editor containing the Bama pig PERV reverse transcriptase with 4 point mutations in PFF.

FIG. 9 shows the editing efficiency of pPE editor containing the Bama pig PERV reverse transcriptase with 4 point mutations in HEK293T, dog fetal fibroblasts (DFF), and sheep fetal fibroblasts (SFF).

FIG. 10 shows the editing efficiency of epPE editor containing the Bama pig PERV reverse transcriptase with 4 point mutations in PFF.

FIG. 11 shows the editing efficiency of epPE editor containing the Bama pig PERV reverse transcriptase with 4 point mutations in HEK293T, dog fetal fibroblasts (DFF), and sheep fetal fibroblasts (SFF).

FIG. 12 shows the editing efficiency of pPE editor containing Bama pig PERV reverse transcriptase with 1-6 point mutations when co-transfected with the fluorescent reporter gene in HEK293T cells.

FIG. 13 shows the editing efficiency of pPE editor containing the Bama pig PERV reverse transcriptase based on M5.1 together with different point mutations in HEK293T cells.

FIG. 14 shows the editing efficiency of pPE editor containing the Bama pig PERV reverse transcriptase based on 7 point mutations in HEK293T cells.

FIG. 15 shows the editing efficiency of epPE editor containing the Bama pig PERV reverse transcriptase based on 7 point mutations in HEK293T cells.

FIG. 16 shows the efficiency of epPE editor containing the Bama pig PERV reverse transcriptase based on 7 point mutations versus PEmax, PE6c and PE6d.

FIG. 17 shows that Csy4 and tCsy4-pegRNA improve the editing efficiency of the PE editor.

FIG. 18 shows that the small molecule compounds improve the editing efficiency of the PE editor.

### DETAILED DESCRIPTION OF THE INVENTION

### I. Definitions

In the present disclosure, unless otherwise specified, the scientific and technical terms used herein have the meanings commonly understood by those skilled in the art. Moreover, the terms related to protein and nucleic acid chemistry, molecular biology, cell and tissue culture, microbiology, immunology and laboratory operation steps used herein are terms and conventional steps widely used in the corresponding fields. For example, standard recombinant DNA and molecular cloning techniques used in the present invention are well known to those skilled in the art and are more fully described in Sambrook, J., Fritsch, E. F. and Maniatis, T., Molecular Cloning: A Laboratory Manual; Cold Spring Harbor Laboratory Press: Cold Spring Harbor, 1989 (hereinafter “Sambrook”). Meanwhile, in order to better understand the present disclosure, definitions and explanations of related terms are provided below.

As used herein, the term “and/or” encompasses all combinations of the items joined by the term, and it is recognized that each combination has been listed individually herein. For example, “A and/or B” encompasses “A,” “A and B,” and “B.” For example, “A, B, and/or C” encompasses “A”, “B”, “C”, “A and B”, “A and C”, “B and C”, and “A and B and C”.

When the term “comprising” is used herein to describe a sequence of a protein or a nucleic acid, the protein or nucleic acid may consist of the sequence, or may have additional amino acids or nucleotides at one or both ends of the protein or nucleic acid, but still have the activities described in the present invention. In addition, it is clear to those skilled in the art that methionine encoded by the start codon at the N-terminus of the polypeptide will be retained under certain practical conditions (for example, when expressed in a specific expression system), but will not substantially affect the function of the polypeptide. Therefore, in the description and claims of the present application, when describing a specific polypeptide amino acid sequence, although it may not contain a methionine at the N-terminus encoded by a start codon, the sequence containing the methionine is also covered herein, and accordingly, the encoding nucleotide sequence may also contain a start codon; and vice versa.

“Genome” as used herein encompasses not only chromosomal DNA present in the nucleus, but also organelle DNA present in subcellular components (e.g., mitochondria, plastid) of a cell.

As used herein, “organism” includes any organism suitable for genome editing, preferably a eukaryote. Examples of organisms include, but are not limited to, mammals such as humans, mice, rats, monkeys, dogs, pigs, sheep, cattle, cats; poultry such as chickens, ducks, geese; plants including monocotyledonous plants and dicotyledonous plants such as rice, corn, wheat, sorghum, barley, soybean, peanut, Arabidopsis thaliana, etc.

“Genetically modified organism” means an organism comprising an exogenous polynucleotide or comprising a modified gene or expression control sequence within its genome. For example, an exogenous polynucleotide can be stably integrated into the genome of the organism and inherited through successive generations. The exogenous polynucleotide may be integrated into the genome alone or as part of a recombinant DNA construct. A modified gene or expression control sequence is a gene or expression control sequence comprising one or more nucleotide substitutions, deletions or additions in the genome of an organism.

“Exogenous” with respect to a sequence means a sequence from a foreign species or, if from the same species, a sequence that has undergone a significant change in composition and/or locus from its native form by deliberate human intervention.

“Polynucleotide,” “nucleic acid sequence,” “nucleotide sequence,” or “nucleic acid fragment” can be used interchangeably and is a single-stranded or double-stranded RNA or DNA polymer, optionally may contain synthetic, non-natural, or altered nucleotide bases. Nucleotides are referred to by their single letter designation as follows: “A” is adenosine or deoxyadenosine (corresponding to RNA or DNA, respectively), “C” represents cytidine or deoxycytidine, “G” represents guanosine or deoxyguanosine, “U” represents uridine, “T” represents deoxythymidine, “R” represents purine (A or G), “Y” represents pyrimidine (C or T), “K” represents G or T, “H” represents A or C or T, “D” represents A, T or G, “I” represents inosine, and “N” represents any nucleotide. Although the nucleotide sequence herein may be represented as a DNA sequence (comprising T), when referring to RNA, those skilled in the art can readily determine the corresponding RNA sequence (i.e. replace T with U).

“Polypeptide,” “peptide,” and “protein” are used interchangeably herein to refer to a polymer of amino acid residues. The term applies to amino acid polymers in which one or more amino acid residues are an artificially chemical analog of a corresponding naturally occurring amino acid, as well as to polymers of naturally occurring amino acids. The terms “polypeptide”, “peptide”, “amino acid sequence” and “protein” may also include modified forms, including but not limited to glycosylation, lipid linking, sulfation, gamma carboxylation of glutamic acid residues, hydroxylation, and ADP-ribosylation.

The sequence “identity” has the meaning recognized in the art, and the sequence identity percentage between two nucleic acid or polypeptide molecules or regions can be calculated using disclosed techniques. Sequence identity can be measured along the full length of a polynucleotide or polypeptide or along a region of the molecule. (See, for example: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there are many ways to measure the identity between two polynucleotides or polypeptides, the term “identity” is well known to the skilled artisan (Carrillo, H. & Lipman, D., SIAMJ Applied Math 48:1073 (1988)).

As used herein, “amino acid position refers to SEQ ID NO: x” (SEQ ID NO: x is a specific sequence listed herein) means that the position number of the specific amino acid described is the position number of the corresponding amino acid on SEQ ID NO: x. The correspondence of amino acids in different sequences can be determined according to sequence alignment methods well known in the art. For example, amino acid correspondence can be determined by the online alignment tool of EMBL-EBI (https:/www.ebi.ac.uk/Tools/psai), where the two sequences can be aligned using the Needleman-Wunsch algorithm using default parameters. For example, if the amino acid alanine at position 360 from the N-terminus of a polypeptide is aligned with the amino acid at position 365 of SEQ ID NO: x in sequence alignment, the amino acid in the polypeptide can also be described herein as “alanine at position 365 of the polypeptide, with the amino acid position referring to SEQ ID NO: x”.

As used herein, “expression construct” refers to a vector, such as a recombinant vector, suitable for expression of a nucleotide sequence of interest in an organism. “Expression” refers to the production of a functional product. For example, expression of a nucleotide sequence may refer to transcription of the nucleotide sequence (e.g., transcription to generate mRNA or functional RNA) and/or translation of RNA into a precursor or mature protein.

The “expression construct” of the present invention may be a linear nucleic acid fragment, a circular plasmid, a viral vector, or, in some embodiments, may be an RNA (such as mRNA) capable of translation, for example, an RNA generated by in vitro transcription. If used in mammals such as humans, the expression construct is preferably a viral vector, e.g., adeno-associated virus (AAV), lentivirus, adenovirus vector, most preferably an AAV vector.

An “expression construct” of the present invention may comprise regulatory sequences and nucleotide sequences of interest from different sources, or regulatory sequences and nucleotide sequences of interest from the same source but arranged in a different manner than normally found in nature.

A “promoter” refers to a nucleic acid fragment capable of controlling transcription of another nucleic acid fragment. In some embodiments of the invention, the promoter is a promoter capable of controlling gene transcription in a cell, whether or not it is derived from the cell. The promoter may be a constitutive promoter or a tissue-specific promoter or a developmentally regulated promoter or an inducible promoter.

Examples of promoters include, but are not limited to, polymerase pol I, pol II, or pol III promoters. Examples of pol I promoters include the chicken RNA pol I promoter. Examples of pol II promoters include, but are not limited to, the cytomegalovirus immediate early (CMV) promoter, the Rous sarcoma virus long terminal repeat (RSV-LTR) promoter, and the simian virus 40 (SV40) immediate early promoter. Examples of pol III promoters include the U6, U3, and H1 promoters. Inducible promoters such as metallothionein promoters may be used. Other examples of promoters include the T7 phage promoter, the T3 phage promoter, the β-galactosidase promoter, and the Sp6 phage promoter. When used in plants, the promoter may be a cauliflower mosaic virus 35S promoter, a corn Ubi-1 promoter, a wheat U6 promoter, a rice U3 promoter, a corn U3 promoter, a rice actin promoter.

“Introducing” a nucleic acid molecule (e.g., plasmid, linear nucleic acid fragment, RNA, etc.) or a protein into an organism refers to transforming an organism cell with the nucleic acid or protein such that the nucleic acid or protein is capable of functioning in the cell. As used herein, “transformation” includes both stable transformation and transient transformation. “Stable transformation” refers to the introduction of exogenous nucleotide sequences into the genome, resulting in stable inheritance of exogenous genes. Once stably transformed, the exogenous nucleic acid sequence is stably integrated into the genome of the organism and any successive generations thereof “Transient transformation” refers to the introduction of a nucleic acid molecule or protein into a cell to perform a function without the stable inheritance of exogenous genes. In transient transformation, exogenous nucleic acid sequences do not integrate into the genome.

A “trait” refers to a physiological, morphological, biochemical, or physical characteristic of a cell or organism.

### II. Reverse Transcriptase from PERV and Variants Thereof

In one aspect, the present invention provides a reverse transcriptase from Porcine Endogenous Retrovirus (PERV) or a functional variant thereof. The reverse transcriptase has reverse transcription activity for generating a complementary DNA strand with a single-stranded RNA as a template, while its functional variant substantially retains or has enhanced reverse transcription activity. The reverse transcriptase from PERV or functional variant thereof of the present invention can be used for prime editing, especially for prime editing in mammalian cells.

In some embodiments, the reverse transcriptase from PERV (also referred to herein as PERV reverse transcriptase) is from type A, type B, or type C PERV, or from a recombinant of different types of PERV, such as a type A/C PERV recombinant.

In some embodiments, the PERV is PERV isolated from Bama miniature pigs. In some embodiments, the PERV is isolated from pigs of other breeds.

In some embodiments, the PERV reverse transcriptase or a functional variant thereof comprises an amino acid sequence of one of SEQ ID NOs: 1-5 or an amino acid sequence having at least 75%, at least 80%, at least 90%, at least 95%, at least 96%, at least 97%, at least 98%, at least 99%, or even 100% sequence identity to one of SEQ ID NOs: 1-5.

In some embodiments, the functional variant of the PERV reverse transcriptase has the RNase H domain deleted. The RNase H domain of the PERV reverse transcriptase, for example, comprises an amino acid sequence corresponding to positions from about 514 to about 616 of SEQ ID NO: 1.

In some embodiments, the functional variant of the PERV reverse transcriptase comprises an amino acid substitution at one or more positions selected from position 63, position 199, position 222, position 305, position 312, position 329, position 330, position 331, position 332, position 408, or position 602.

When referring to the amino acid position of PERV reverse transcriptase or functional variant thereof herein, the amino acid position refers to SEQ ID NO: 5.

In some embodiments, the functional variant of the PERV reverse transcriptase comprises amino acid substitutions at positions 199, 305, and 312. In some embodiments, the functional variant of the PERV reverse transcriptase comprises amino acid substitutions D199N, T305K, and W312F.

In some embodiments, the functional variant of the PERV reverse transcriptase comprises amino acid substitutions at positions 199, 305, 312, and 602. In some embodiments, the functional variant of the PERV reverse transcriptase comprises amino acid substitutions D199N, T305K, W312F, and L602W.

In some embodiments, the functional variant of the PERV reverse transcriptase comprises amino acid substitutions at positions 199, 305, 312, 329, 330, 331, and 332. In some embodiments, the functional variant of the PERV reverse transcriptase comprises amino acid substitutions D199N, T305K, W312F, E329P, K330G, G331T, and E332C.

In some embodiments, the functional variant of the PERV reverse transcriptase comprises amino acid substitutions at positions 199, 305, 312, 329, 330, 331, 332, and 602. In some embodiments, the functional variant of the PERV reverse transcriptase comprises amino acid substitutions D199N, T305K, W312F, E329P, K330G, G331T, E332C, and L602W.

In some embodiments, the functional variant of the PERV reverse transcriptase comprises amino acid substitutions at positions 199, 222, 305, 312, and 602. In some embodiments, the functional variant of the PERV reverse transcriptase comprises amino acid substitutions D199N, V222A, T305K, W312F, and L602W.

In some embodiments, the functional variant of the PERV reverse transcriptase comprises amino acid substitutions at positions 63, 199, 222, 305, 312, and 602. In some embodiments, the functional variant of the PERV reverse transcriptase comprises amino acid substitutions Y63R, D199N, V222A, T305K, W312F, and L602W.

In some embodiments, the functional variant of the PERV reverse transcriptase comprises amino acid substitutions at positions 199, 222, 305, 312, 408, and 602. In some embodiments, the functional variant of the PERV reverse transcriptase comprises amino acid substitutions D199N, V222A, T305K, W312F, C408R, and L602W.

In some embodiments, the functional variant of the PERV reverse transcriptase comprises an amino acid substitution at positions 63, 199, 222, 305, 312, 408, and 602. In some embodiments, the functional variant of the PERV reverse transcriptase comprises amino acid substitutions Y63R, D199N, V222A, T305K, W312F, C408R, and L602W.

In some embodiments, the functional variant of the PERV reverse transcriptase comprises an amino acid sequence selected from the group consisting of SEQ ID NO: 6 (corresponding to AC-RT-M0Δ), SEQ ID NO: 7 (corresponding to AC-RT-M3Δ), SEQ ID NO: 8 (corresponding to AC-RT-M4Δ*), SEQ ID NO: 9 (corresponding to B-RT-M0Δ), SEQ ID NO: 10 (corresponding to B-RT-M3Δ), SEQ ID NO: 11 (corresponding to B-RT-M4Δ*), and SEQ ID NO: 12 (corresponding to bmx-A-RT-M4), SEQ ID NO: 13 (corresponding to bmx-B-RT-M4), SEQ ID NO: 14 (corresponding to bmx-C-RT-M4), SEQ ID NO: 15 (corresponding to bmx-A-RT-M3Δ), SEQ ID NO: 16 (corresponding to bmx-B-RT-M3Δ), SEQ ID NO: 17 (corresponding to bmx-C-RT-M3Δ), SEQ ID NO: 40 (corresponding to bmx-C-RT-M5.1), SEQ ID NO: 41 (corresponding to bmx-C-RT-M5.1+Y63R), SEQ ID NO: 42 (corresponding to bmx-C-RT-M5.1+C408R), SEQ ID NO: 43 (corresponding to bmx-C-RT-M7), and SEQ ID NO: 44 (corresponding to bmx-C-RT-M7Δ).

In another aspect, the present invention provides use of the reverse transcriptase from PERV or a functional variant thereof according to the present invention in prime editing, especially in prime editing in a mammalian cell.

As used herein, “mammal” includes, but is not limited to, human, mouse, rat, monkey, dog, pig, sheep, cow, cat, etc. In some preferred embodiments, the mammal is a pig. In some preferred embodiments, the mammal is a human.

### III. Prime Editing System Based on Reverse Transcriptase from PERV

In one aspect, the present invention relates to a prime editing system for targeted modification of a genomic DNA sequence of an organism, comprising:
i) a CRISPR nuclease such as a CRISPR nickase and/or an expression construct comprising a nucleotide sequence encoding said CRISPR nuclease such as a CRISPR nickase, and a PERV reverse transcriptase or a functional variant thereof and/or an expression construct comprising a nucleotide sequence encoding said PERV reverse transcriptase or a functional variant thereof; and/or
ii) at least one pegRNA and/or an expression construct comprising a nucleotide sequence encoding the at least one pegRNA.

In some embodiments, wherein the at least one pegRNA comprises, from 5′ to 3′ direction, a leader sequence, a scaffold sequence, a reverse transcription template (RTT) sequence, and a primer binding site (PBS) sequence.

In some embodiments, the CRISPR nuclease such as CRISPR nickase and the PERV reverse transcriptase or a functional variant thereof form a fusion protein.

In one aspect, the present invention relates to a prime editing system for targeted modification of a genomic DNA sequence of an organism, comprising:
i) a prime editing fusion protein and/or an expression construct comprising a nucleotide sequence encoding the prime editing fusion protein, wherein the prime editing fusion protein comprises a CRISPR nuclease such as CRISPR nickase and PERV reverse transcriptase or a functional variant thereof; and/or
ii) at least one pegRNA and/or an expression construct comprising a nucleotide sequence encoding the at least one pegRNA.

In some embodiments, wherein the at least one pegRNA comprises, from 5′ to 3′ direction, a leader sequence, a scaffold sequence, a reverse transcription template (RTT) sequence, and a primer binding site (PBS) sequence.

In another aspect, the present invention also provides a prime editing fusion protein comprising a CRISPR nuclease such as CRISPR nickase and a PERV reverse transcriptase or a functional variant thereof. The present invention also provides use of the prime editing fusion protein in prime editing, especially in prime editing in a mammalian cell.

In some embodiments, the CRISPR nuclease such as CRISPR nickase and the PERV reverse transcriptase or a functional variant thereof in the prime editing fusion protein are linked by a linker.

In some embodiments, the at least one pegRNA is capable of forming a complex with the CRISPR nuclease, such as a CRISPR nickase, the PERV reverse transcriptase or a functional variant thereof and targeting the fusion protein to a target sequence in the genome, resulting in a nick within the target sequence.

In some embodiments, the at least one pegRNA is capable of forming a complex with the fusion protein and targeting the fusion protein to a target sequence in the genome, resulting in a nick within the target sequence.

As used herein, a “prime editing system” refers to a combination of components required for reverse transcription-based genome editing of a genome in a cell. The components of the system, for example, CRISPR nuclease such as CRISPR nickase, PERV reverse transcriptase or functional variants thereof, prime editing fusion protein, pegRNA, and the like, may each be present independently, or may be present in any combination in the form as a composition.

As used herein, a “target sequence” refers to a sequence of about 20 nucleotides in length characterized by a 5′ or 3′ flanking PAM (Protospacer Adjacent Motif) sequence in the genome. In general, PAM is necessary for a complex formed by a CRISPR nuclease or a variant thereof and a guide RNA to recognize a target sequence. For example, for a Cas9 nuclease and variant thereof, its target sequence is immediately adjacent to PAM at the 3′ end, such as 5′-NGG-3′. Based on the presence of PAM, one skilled in the art can readily determine the target sequences available for targeting in the genome. Also depending on the location of the PAM, the target sequence may be located on either strand of the genomic DNA molecule. For Cas9 or its derivatives such as Cas9 nickase, the target sequence is preferably of 20 nucleotides.

The CRISPR nuclease of the invention may be a CRISPR nuclease of different origin. Examples of CRISPR nucleases suitable for the present invention include, but are not limited to, Cas9, Cas12a (i.e. Cpf1), Cas12b (i.e. C2c1), Cas12c, Cas12d (i.e. CasY), Cas12e (i.e. CasX), Cas12f (i.e. Cas14), Cas12h, Cas12i, Cas12j (i.e. Cas Φ), Cas12k, Cas12m, IsrB, IscB, and TnpB.

In some embodiments, the CRISPR nuclease also encompasses the nickase form thereof. A nickase form of a CRISPR nuclease may also be referred to herein as a CRISPR nickase, which forms a nick in only one strand of a double-stranded nucleic acid molecule, but does not completely cleave the double-stranded nucleic acid. In some embodiments, the CRISPR nickase is capable of forming a nick within a target sequence in the genomic DNA. One skilled in the art can obtain nickase forms of different CRISPR nucleases, for example by mutating the nucleic acid cleavage domain of the CRISPR nuclease.

In some embodiments, the CRISPR nickase is a Cas9 nickase. However, CRISPR nickase may also be derived from other CRISPR nucleases.

In some embodiments, the Cas9 nickase is derived from SpCas9 of S. pyogenes and comprises at least the amino acid substitution H840A relative to wild-type SpCas9. In some embodiments, the Cas9 nickase comprises the amino acid sequence shown in SEQ ID NO: 18. In some embodiments, the Cas9 nickase in the fusion protein can form a nick between nucleotide −3 and nucleotide −4 from the PAM of the target sequence (the first nucleotide at the 5′ end of the PAM sequence is +1).

In some embodiments, the Cas9 nickase is a Cas9 nickase variant capable of recognizing an altered PAM sequence. Many Cas9 nickase variants capable of recognizing altered PAM sequences are known in the art. For example, the Cas9 nickase is a Cas9 variant that recognizes the PAM sequence 5′-NG-3′. These various variants may be applied in the present invention.

The nick formed by the Cas9 nickase of the present invention can cause the target sequence to form a free single strand having a 3′ end (3′ free single strand) and a free single strand having a 5′ end (5′ free single strand).

In some embodiments, the PERV reverse transcription or the functional variant thereof in the fusion protein is the PERV reverse transcriptase or the functional variant thereof described above in the present invention.

In some embodiments, the PERV reverse transcriptase is from type A, type B, or type C PERV, or from a recombinant of different types of PERV, such as A/C PERV recombinant.

In some embodiments, the PERV is PERV isolated from Bama pigs. In some embodiments, the PERV is isolated from pigs of other breeds.

In some embodiments, the PERV reverse transcriptase or a functional variant thereof comprises an amino acid sequence of one of SEQ ID NOs: 1-5 or an amino acid sequence having at least 75%, at least 80%, at least 90%, at least 95%, at least 96%, at least 97%, at least 98%, at least 99%, or even at least 100% sequence identity to one of SEQ ID NOs: 1-5.

In some embodiments, the functional variant of the PERV reverse transcriptase has the RNase H domain deleted. The RNase H domain of the PERV reverse transcriptase, for example, comprises an amino acid sequence corresponding to positions from about 514 to about 616 of SEQ ID NO: 5.

In some embodiments, the functional variant of the PERV reverse transcriptase comprises an amino acid substitution at one or more positions selected from position 199, position 305, position 312, position 329, position 330, position 331, position 332, or position 602.

When referring to the amino acid position of PERV reverse transcriptase or functional variant thereof herein, the amino acid position refers to SEQ ID NO: 1.

In some embodiments, the functional variant of the PERV reverse transcriptase comprises amino acid substitutions at positions 199, 305, and 312. In some embodiments, the functional variant of the PERV reverse transcriptase comprises amino acid substitutions D199N, T305K, and W312F.

In some embodiments, the functional variant of the PERV reverse transcriptase comprises amino acid substitutions at positions 199, 305, 312, and 602. In some embodiments, the functional variant of the PERV reverse transcriptase comprises amino acid substitutions D199N, T305K, W312F, and L602W.

In some embodiments, the functional variant of the PERV reverse transcriptase comprises amino acid substitutions at positions 199, 305, 312, 329, 330, 331, and 332. In some embodiments, the functional variant of the PERV reverse transcriptase comprises amino acid substitutions D199N, T305K, W312F, E329P, K330G, G331T, and E332C.

In some embodiments, the functional variant of the PERV reverse transcriptase comprises amino acid substitutions at positions 199, 305, 312, 329, 330, 331, 332, and 602. In some embodiments, the functional variant of the PERV reverse transcriptase comprises amino acid substitutions D199N, T305K, W312F, E329P, K330G, G 331T, E332C, and L602W.

In some embodiments, the functional variant of the PERV reverse transcriptase comprises amino acid substitutions at positions 199, 222, 305, 312, and 602. In some embodiments, the functional variant of the PERV reverse transcriptase comprises amino acid substitutions D199N, V222A, T305K, W312F, and L602W.

In some embodiments, the functional variant of the PERV reverse transcriptase comprises amino acid substitutions at positions 63, 199, 222, 305, 312, and 602. In some embodiments, the functional variant of the PERV reverse transcriptase comprises amino acid substitutions Y63R, D199N, V222A, T305K, W312F, and L602W.

In some embodiments, the functional variant of the PERV reverse transcriptase comprises amino acid substitutions at positions 199, 222, 305, 312, 408, and 602. In some embodiments, the functional variant of the PERV reverse transcriptase comprises amino acid substitutions D199N, V222A, T305K, W312F, C408R, and L602W.

In some embodiments, the functional variant of the PERV reverse transcriptase comprises an amino acid substitution at positions 63, 199, 222, 305, 312, 408, and 602. In some embodiments, the functional variant of the PERV reverse transcriptase comprises amino acid substitutions Y63R, D199N, V222A, T305K, W312F, C408R, and L602W.

In some embodiments, the functional variant of the PERV reverse transcriptase comprises an amino acid sequence selected from the group consisting of SEQ ID NO: 6 (corresponding to AC-RT-M0Δ), SEQ ID NO: 7 (corresponding to AC-RT-M3Δ), SEQ ID NO: 8 (corresponding to AC-RT-M4Δ*), SEQ ID NO: 9 (corresponding to B-RT-M0Δ), SEQ ID NO: 10 (corresponding to B-RT-M3Δ), SEQ ID NO: 11 (corresponding to B-RT-M4Δ*), and SEQ ID NO: 12 (corresponding to bmx-A-RT-M4), SEQ ID NO: 13 (corresponding to bmx-B-RT-M4), SEQ ID NO: 14 (corresponding to bmx-C-RT-M4), SEQ ID NO: 15 (corresponding to bmx-A-RT-M3Δ), SEQ ID NO: 16 (corresponding to bmx-B-RT-M3Δ), SEQ ID NO: 17 (corresponding to bmx-C-RT-M3Δ), SEQ ID NO: 40 (corresponding to bmx-C-RT-M5.1), SEQ ID NO: 41 (corresponding to bmx-C-RT-M5.1+Y63R), SEQ ID NO: 42 (corresponding to bmx-C-RT-M5.1+C408R), SEQ ID NO: 43 (corresponding to bmx-C-RT-M7), and SEQ ID NO: 44 (corresponding to bmx-C-RT-M7Δ).

In some embodiments, in the fusion protein, the reverse transcriptase or a functional variant thereof is fused with a nucleocapsid protein (NC) at the N-terminus or C-terminus, either directly or through a linker. The nucleocapsid protein (NC) is, for example, from PERV. Preferably, the nucleocapsid protein and the reverse transcriptase are from the same type of PERV.

In some embodiments, the nucleocapsid protein comprises an amino acid sequence set forth in one of SEQ ID NO: 19 or SEQ ID NO: 36-39.

In some embodiments, the PERV reverse transcriptase or a functional variant thereof is fused to the nucleocapsid protein (NC) at the N-terminus, either directly or through a linker.

In some embodiments, the PERV reverse transcriptase or a functional variant thereof is fused to the nucleocapsid protein (NC) at the C-terminus, either directly or through a linker.

As used herein, a “linker” may be a non-functional amino acid sequence that is 1-50 (e.g., 1, 2, 3, 4, 5, 6, 7, 8, 9, 10, 11, 12, 13, 14, 15, 16, 17, 18, 19, 20 or 20-25, 25-50) or more amino acids in length, without a secondary or higher structure. For example, the linker may be a flexible linker or the like. For example, it may be the XTEN linker shown in SEQ ID NO: 20.

In some embodiments, the CRISPR nuclease such as CRISPR nickase in the fusion protein is located at the N-terminus of the reverse transcriptase and/or nucleocapsid protein. In some embodiments, the CRISPR nuclease such as CRISPR nickase in the fusion protein is located at the C-terminus of the reverse transcriptase and/or nucleocapsid protein.

In some embodiments of the present invention, the fusion protein of the present invention may further comprise a nuclear localization sequence (NLS). In general, one or more of the NLSs in the fusion protein should be of sufficient strength so as to drive the fusion protein in the nucleus of the cell to accumulate in an amount that can achieve its base editing function. In general, the strength of nuclear localization activity is determined by the number of NLSs in the fusion protein, the location, the particular NLS or NLSs used, or a combination of these factors. However, depending on the location of the DNA to be edited, the prime editing fusion protein of the present invention may also include one or more other localization sequences, such as cytoplasmic localization sequences, chloroplast localization sequences, mitochondrial localization sequences, etc.

In some embodiments, the fusion protein may also include a selectable marker protein linked directly or through a self-cleaving peptide.

“Self-cleaving peptide” as used herein means a peptide that can achieve self-cleavage within a cell. For example, the self-cleaving peptide may comprise a protease recognition site, thereby being recognized and specifically cleaved by a protease within the cell.

Alternatively, the self-cleaving peptide may be a 2A polypeptide. 2A polypeptides are a class of short peptides from viruses whose self-cleavage occurs during translation. When two different proteins of interest are linked with a 2A polypeptide and expressed in the same reading frame, the two proteins of interest are produced at a ratio of almost 1:1. Common 2A polypeptides can be P2A from porcine techovirus-1, T2A from Thosea asigna virus, E2A from equine rhinitis A virus, and F2A from foot-and-mouth disease virus. P2A has the highest cleavage efficiency and thus is preferred. A variety of functional variants of these 2A polypeptides are also known in the art, and these variants can also be used in the present invention.

An exemplary selectable marker protein is, for example, a Puro-R protein. The Puro-R protein can provide puromycin resistance for selecting positive cells. An exemplary Puro-R protein comprises the amino acid sequence set forth in SEQ ID NO: 35.

In some embodiments, the fusion protein comprises an amino acid sequence as set forth in one of SEQ ID NOs: 22-34 and 45-49.

The leader sequence (also referred to as a seed sequence or a spacer sequence) in the at least one pegRNA of the present invention is configured to have sufficient sequence identity (preferably 100% identity) with the target sequence, so that sequence-specific targeting can be achieved by binding to the complementary strand of the target sequence through base pairing.

A variety of scaffold sequences of gRNAs suitable for genome editing based on CRISPR nucleases (e.g., Cas9) are known in the art and can be used in the pegRNAs of the invention. In some specific embodiments, the scaffold sequence of the gRNA is shown in SEQ ID NO: 21.

In some embodiments, the primer binding sequence (PBS) is configured to be complementary to at least a part of the target sequence (preferably fully paired with at least a part of the target sequence), preferably, the primer binding sequence is complementary to at least a part of the 3′ free single strand caused by nicking in the DNA strand where the target sequence is located (preferably fully paired with at least a part of the 3′ free single strand), especially complementary to the nucleotide sequence at the 3′ end of the 3′ free single strand (preferably fully paired). When the 3′ free single strand of the strand binds to the primer binding sequence through base pairing, the 3′ free single strand can be used as a primer, and a reverse transcription template (RTT) sequence immediately adjacent to the primer binding sequence is used as a template, and reverse transcription is performed under the action of a reverse transcriptase or a functional variant thereof in the fusion protein to extend a DNA sequence corresponding to the reverse transcription (RT) template sequence.

The primer binding sequence depends on the length of the free single strand formed in the target sequence by the CRISPR nickase used, however, it should have the minimum length to ensure specific binding. In some embodiments, the primer binding sequence can be 4-20 nucleotides in length, such as 4, 6, 7, 8, 9, 10, 11, 12, 13, 14, 15, 16, 17, 18, 19, 20 nucleotides in length.

The RT template sequence described herein may be any sequence. Through the above reverse transcription, its sequence information can be integrated into the DNA strand where the target sequence is located (i.e., the strand containing the target sequence PAM), and then through the DNA repair effect of the cell, a DNA double strand containing the RT template sequence information is formed. In some embodiments, the RT template sequence comprises a desired modification. For example, the desired modification includes substitution, deletion, and/or addition of one or more nucleotides. For example, the modification comprises one or more substitutions selected from the group consisting of a C to T substitution, a C to G substitution, a C to A substitution, a G to T substitution, a G to C substitution, a G to A substitution, an A to T substitution, an A to G substitution, an A to C substitution, a T to C substitution, a T to G substitution, a T to A substitution; and/or comprises a deletion of one or more nucleotides, such as deletion of 1 to about 100 or more, such as 1, 2, 3, 4, 5, about 10, about 20, about 30, about 40, about 50, about 75, about 100 nucleotides; and/or comprises an insertion of one or more nucleotides, such as insertion of 1 to about 100 or more, such as 1, 2, 3, 4, 5, about 10, about 20, about 30, about 40, about 50, about 75, about 100 nucleotide.

In some embodiments, the RT template sequence is configured to correspond to a sequence downstream the nick of the target sequence (e.g., complementary to at least a portion of the sequence downstream the nick of the target sequence) and comprises a desired modification. The desired modification includes substitution, deletion and/or addition of one or more nucleotides. For example, the modification comprises one or more substitutions selected from the group consisting of a C to T substitution, a C to G substitution, a C to A substitution, a G to T substitution, a G to C substitution, a G to A substitution, an A to T substitution, an A to G substitution, an A to C substitution, a T to C substitution, a T to G substitution a T to A substitution; and/or comprises a deletion of one or more nucleotides, such as deletion of 1 to about 100 or more, such as 1, 2, 3, 4, 5, about 10, about 20, about 30, about 40, about 50, about 75, about 100 nucleotides; and/or comprises an insertion of one or more nucleotides, such as insertion of 1 to about 100 or more, such as 1, 2, 3, 4, 5, about 10, about 20, about 30, about 40, about 50, about 75, about 100 nucleotide.

In some embodiments, the RT template sequence may be about 1-300 nucleotides or more in length, such as 1, 2, 3, 4, 5, about 10, about 20, about 30, about 40, about 50, about 75, about 100, about 125, about 150, about 175, about 200, about 225, about 250, about 275, about 300 or more nucleotides in length. Preferably, the RT template sequence is 7, 8, 9, 10, 11, 12, 13, 14, 15, 16, 17, 18, 19, 20, 21, 22, or 23 nucleotides in length.

In some embodiments, the prime editing system further comprises a nicking gRNA (for generating an additional nick) comprising a lead sequence and a scaffold sequence and/or an expression construct comprising a nucleotide sequence encoding the nicking gRNA. In some preferred embodiments, the nicking gRNA does not comprise a reverse transcription (RT) template sequence or a primer binding site (PBS) sequence.

The leader sequence (also referred to as seed sequence or spacer sequence) in the nicking gRNA of the present invention is configured to have sufficient sequence identity (preferably 100% identity) with a nicking target sequence in the genome, so that the fusion protein of the present invention can be targeted to the nicking target sequence and result in a nick within the nicking target sequence, and the nicking target sequence and the pegRNA-targeted target sequence (pegRNA target sequence) are located on opposite strands of the genomic DNA. In some embodiments, the nick formed by the nicking gRNA and the nick formed by the pegRNA are about 1 to about 300 or more nucleotides apart, such as 1, 2, 3, 4, 5, about 10, about 20, about 30, about 40, about 50, about 75, about 100, about 125, about 150, about 175, about 200, about 225, about 250, about 275, about 300 or more nucleotides apart. In some embodiments, the nick formed by the nicking gRNA is located upstream or downstream of the formation of the pegRNA (the upstream or downstream refer to the DNA strand where the target sequence of the pegRNA is located). In some embodiments, the leader sequence in the nicking gRNA and the opposite strand (modified) of the pegRNA target sequence after the editing event occurs have sufficient sequence identity (preferably 100% identity), so that the nicking gRNA only targets the nicking target sequence generated after the pegRNA-induced target sequence targeting and modification. In some embodiments, the PAM of the nicking target sequence is located within the complement sequence of the pegRNA target sequence.

In some embodiments, the prime editing system comprises at least one pair of pegRNAs and/or an expression construct comprising a nucleotide sequence encoding the at least one pair of pegRNAs. In some embodiments, two pegRNAs of the pair of pegRNAs are configured to target different target sequences on the same strand of genomic DNA. In some embodiments, two pegRNAs of the pair of pegRNAs are configured to target the target sequences on different strands of genomic DNA. In some embodiments, the PAM of the target sequence of one pegRNA of the pair of pegRNAs is located on the sense strand and the PAM of the other pegRNA is located on the antisense strand. In some embodiments, the induced nicks of the two pegRNAs are located on both sides of the site to be modified, respectively. In some embodiments, the pegRNA-induced nick for the sense strand is located upstream (5′ direction) of the site to be modified, and the pegRNA-induced nick for the antisense strand is located downstream (3′ direction) of the site to be modified. The upstream or downstream is relative to the sense chain. In some embodiments, the induced nicks of the two pegRNAs are about 1 to about 300 or more nucleotides apart, for example 1-15 nucleotides apart.

In some embodiments, both pegRNAs of the pair of pegRNAs are configured to introduce the same desired modification. For example, one of the pegRNAs is configured to introduce A to G substitutions at the sense strand, and the other pegRNA is configured to introduce T to C substitutions at the corresponding positions of the antisense strand. For another example, one pegRNA is configured to introduce a deletion of two nucleotides at the sense strand, and the other pegRNA is configured to also introduce a deletion of two nucleotides at the corresponding position of the antisense strand. Other types of modifications may be deduced similarly. It can allow two pegRNAs targeting different strands to achieve a same desired modification by designing suitable RT template sequences.

In some preferred embodiments, the pegRNA of the present invention is an epegRNA. The construction of epegRNA can be found in James W. Nelson et al., Engineered pegRNAs improve prime seeding efficiency. Nature Biotechnology volume 40, 402-410 (2022), which is incorporated herein by reference. In some embodiments, the epegRNA is an epegRNA with a 3′-tevopreQ1-8 nt linker modification.

In some preferred embodiments, the pegRNA of the present invention is Csy4-pegRNA. The Csy4-pegRNA comprises a Csy4 recognition sequence at the 3′ end of the pegRNA, such as the Csy4 recognition sequence shown in SEQ ID NO: 50.

In some preferred embodiments, the pegRNA of the present invention is tCsy4-pegRNA. The tCsy4-pegRNA comprises a truncated Csy4 recognition sequence at the 3′ end of the pegRNA, such as the truncated Csy4 recognition sequence shown in SEQ ID NO: 51.

In order to obtain effective expression in different organisms, in some embodiments of the present invention, the nucleotide sequence encoding the fusion protein is codon optimized for the organism species whose genome is to be modified.

Codon optimization refers to a method of modifying a nucleic acid sequence to enhance expression in a host cell of interest by replacing at least one codon of a native sequence (e.g., about or more than about 1, 2, 3, 4, 5, 10, 15, 20, 25, 50 or more codons) with a codon more frequently or most frequently used in a gene of the host cell while maintaining the native amino acid sequence. Different species exhibit specific preferences for certain codons of specific amino acids. Codon preference, the difference in codon usage between organisms, is often associated with the translation efficiency of messenger RNA (mRNA), which is believed to depend on the nature of the codon being translated and the availability of a particular transport RNA (tRNA) molecule. The advantages of selected tRNAs in cells generally reflect the codons most frequently used for peptide synthesis. Thus, genes can be tailored to optimize gene expression in a given organism based on codon. Codon utilization tables are readily available, for example, in the Codon Usage Database (“Codon Usage Database”) available at www.kazusa. See, Nakamura Y et al., “Codon usage tabulated from the international DNA sequence databases: status for the year 2000. Nucl. Acids Res., 28:292 (2000).

In another aspect, the present invention also provides a truncated Csy4 recognition sequence element having a nucleotide sequence as shown in SEQ ID NO: 51. In another aspect, the invention also provides a nucleic acid molecule, such as an RNA molecule, comprising the truncated Csy4 recognition sequence element. The RNA molecule is, for example, gRNA, preferably pegRNA. The gRNA and/or pegRNA are as defined above. In another aspect, the present invention also provides use of the truncated Csy4 recognition sequence element in gene editing.

### IV. Method for Modifying the Genomic Sequence of a Cell

In another aspect, the present invention provides a method for modifying the genomic sequence of a cell, comprising introducing the prime editing system of the present invention into at least one cell, thereby resulting in modification of the genomic sequence of the at least one cell. The modification includes substitution, deletion and/or addition of one or more nucleotides. For example, the modification comprises one or more substitutions selected from the group consisting of a C to T substitution, a C to G substitution, a C to A substitution, a G to T substitution, a G to C substitution, a G to A substitution, an A to T substitution, an A to G substitution, an A to C substitution, a T to C substitution, a T to G substitution, a T to A substitution; and/or a deletion of one or more nucleotides, such as deletion of 1 to about 100 or more, such as 1, 2, 3, 4, 5, about 10, about 20, about 30, about 40, about 50, about 75, about 100 nucleotides; and/or an insertion of one or more nucleotides, such as insertion of 1 to about 100 or more, such as 1 to about 100 or more, such as 1, 2, 3, 4, 5, about 10, about 20, about 30, about 40, about 50, about 75, about 100 nucleotides. The modification may be located in the target sequence of the pegRNA or near the target sequence of the pegRNA, e.g., downstream of the target sequence.

In some embodiments, the prime editing system is introduced into the cell in the presence of one or more small molecule compounds selected from RS-1, Nocodazole (Noc) and Trichostatin A (TSA) (preferably a combination of RS-1 and TSA or nocodazole, more preferably a combination of RS-1 and nocodazole).

In some embodiments, after the prime editing system is introduced into the cell, the cell is treated with one or more small molecule compounds selected from RS-1, Nocodazole (Noc) and Trichostatin A (TSA) (preferably a combination of RS-1 and TSA or nocodazole, more preferably a combination of RS-1 and nocodazole), for example, the cell is cultured in a medium comprising the one or more small molecule compounds (preferably a combination of RS-1 and TSA or nocodazole, more preferably a combination of RS-1 and nocodazole).

The concentration of nocodazole may range from about 50 ng/ml to about 500 ng/ml, for example from about 100 ng/ml to about 300 ng/ml, preferably about 200 ng/mL. The concentration of RS-1 may be about 5 μM to about 50 μM, for example, about 10 μM to about 30 μM, preferably about 20 μM. The concentration of trichostatin A may be from about 1 nM to about 50 nM, for example from about 5 nM to about 20 nM, preferably about 10 nM.

In another aspect, the invention also provides a method for producing a genetically modified cell comprising introducing the prime editing system of the invention into the cell.

The genetic modification includes substitution, deletion and/or addition of one or more nucleotides. For example, the genetic modification comprises one or more substitutions selected from the group consisting of a C to T substitution, a C to G substitution, a C to A substitution, a G to T substitution, a G to C substitution, a G to A substitution, an A to T substitution, an A to G substitution, an A to C substitution, a T to C substitution, a T to G substitution, a T to A substitution; and/or a deletion of one or more nucleotides, such as deletion of 1 to about 100 or more, such as 1, 2, 3, 4, 5, about 10, about 20, about 30, about 40, about 50, about 75, about 100 nucleotides; and/or an insertion of one or more nucleotides, such as insertion of 1 to about 100 or more, such as 1 to about 100 or more, such as 1, 2, 3, 4, 5, about 10, about 20, about 30, about 40, about 50, about 75, about 100 nucleotides. The modification may be located in the target sequence of the pegRNA or near the target sequence of the pegRNA, e.g., downstream of the target sequence.

In some embodiments, the prime editing system is introduced into the cell in the presence of one or more small molecule compounds selected from RS-1, Nocodazole (Noc) and Trichostatin A (TSA), preferably RS-1 and nocodazole.

In some embodiments, after the prime editing system is introduced into the cell, the cell is treated with one or more small molecule compounds selected from RS-1, Nocodazole (Noc) and Trichostatin A (TSA) (preferably RS-1 and nocodazole), for example, the cell is cultured in a medium comprising the one or more small molecule compounds (preferably RS-1 and nocodazole).

In another aspect, the invention also provides a genetically modified organism comprising the genetically modified cell produced by the method of the invention or progeny cell thereof.

In the present invention, the modification may be located at any position in the genome, for example, in a functional gene such as a protein encoding gene, or may be located in a gene expression regulatory region such as a promoter region or an enhancer region, so as to realize the functional modification of the gene or the modification of the gene expression. The modification in the genomic sequence of the cell can be detected by T7EI, PCR/RE or sequencing methods.

In the method of the present invention, the prime editing system can be introduced into the cell by various methods well known to those skilled in the art.

Methods that can be used to introduce the prime editing system of the present invention into a cell include, but are not limited to, calcium phosphate transfection, protoplast fusion, electroporation, liposome transfection, microinjection, viral infection (such as baculovirus, vaccinia virus, adenovirus, adeno-associated virus, lentivirus, and other viruses), gene gun method, PEG-mediated protoplast transformation, Agrobacterium-mediated transformation.

Cells that can be genetically edited by the methods of the present invention can be derived from, for example, mammals such as human, mouse, rat, monkey, dog, pig, sheep, cattle, cat; poultry such as chicken, duck, goose; plants, including monocots and dicots, such as rice, corn, wheat, sorghum, barley, soybean, peanuts, Arabidopsis, and the like. In some preferred embodiments, the cell is from human. In some preferred embodiments, the cell is from a pig.

In some embodiments, the method of the present invention is performed in vitro. For example, the cell is an isolated cell, or a cell within an isolated tissue or organ.

In other embodiments, the method of the present invention may also be performed in vivo. For example, the cell is a cell in an organism, and the system of the present invention can be introduced into the cell in vivo by, for example, virus- or Agrobacterium-mediated methods.

In another aspect, the present invention provides use of one or more small molecule compounds selected from RS-1, Nocodazole (Noc) and Trichostatin A (TSA) (preferably a combination of RS-1 and TSA or nocodazole, more preferably a combination of RS-1 and nocodazole) in prime editing, especially in prime editing in a mammalian cell.

### V. Therapeutic Uses

The present invention also encompasses the use of the prime editing system of the present invention in the treatment of a disease.

By modifying a disease-related gene through the prime editing system of the present invention, the up-regulation, down-regulation, inactivation, activation or mutation correction of the disease-related gene can be achieved, thereby achieving the prevention and/or treatment of disease. For example, the genome modification in the present invention may be located in a protein coding region of a disease-related gene, or may be located, for example, in a gene expression regulatory region such as a promoter region or an enhancer region, so that the disease-related gene function modification or the disease-related gene expression modification can be realized. Therefore, the modification of the disease-related gene described herein includes the modification of the disease-related gene itself (e.g., the protein coding region) and also includes the modification of its expression regulation region (e.g., promoter, enhancer, intron, etc.).

A “disease-related” gene refers to any gene that produces a transcription or translation product at an abnormal level or in an abnormal form in a cell derived from a disease-affected tissue as compared to a non-disease control tissue or cell. Where altered expression is associated with the occurrence and/or progression of a disease, it may be a gene expressed at an abnormally high level or it may be a gene expressed at an abnormally low level. A disease-related gene also refers to a gene having one or more mutations or genetic variations that are directly responsible for, or in linkage disequilibrium with one or more genes responsible for the etiology of the disease. The mutation or genetic variation is, for example, a single nucleotide variation (SNV). The transcribed or translated product may be known or unknown and may be at a normal or abnormal level.

Accordingly, the present invention also provides a method for treating a disease in a subject in need thereof, comprising delivering to the subject an effective amount of the prime editing system of the present invention to modify a gene related to the disease. The present invention also provides the use of the prime editing system for modifying a gene related to a disease in a subject in need thereof in the preparation of a pharmaceutical composition for treating the disease. The present invention also provides a pharmaceutical composition for treating a disease in a subject in need thereof, comprising the prime editing system of the present invention for modifying a gene related to the disease, and optionally a pharmaceutically acceptable carrier.

Preferably, the pharmaceutical composition of the present invention further comprises one or more small molecule compounds selected from RS-1, Nocodazole and Trichostatin A (TSA) (preferably a combination of RS-1 and TSA or nocodazole, more preferably a combination of RS-1 and nocodazole). In some other preferred embodiments, the pharmaceutical composition of the present invention is used in combination with one or more small molecule compounds selected from RS-1, Nocodazole and Trichostatin A (TSA) (preferably a combination of RS-1 and TSA or nocodazole, more preferably a combination of RS-1 and nocodazole).

Preferably, the “subject” of the present invention is a mammal, such as a human, mouse, rat, monkey, dog, pig, sheep, bovine or cat, preferably a human.

In some embodiments, the prime editing system described herein is used to introduce a point mutation into a nucleic acid.

In some embodiments, the prime editing system described herein is used to cause correction of a genetic defect, such as in correcting a point mutation in a gene product that cause loss of function. In some embodiments, the genetic defect is associated with a disease or disorder (e.g., a lysosomal storage disease or a metabolic disease, such as type I diabetes). In some embodiments, the method provided herein can be used to introduce a deactivation point mutation into a gene or allele encoding a gene product associated with a disease or disorder.

In some embodiments, the embodiment described herein is intended for the treatment of diseases associated with or caused by point mutations that can be corrected by the prime editing system provided herein. In some embodiments, the disease is a proliferative disease. In some embodiments, the disease is a genetic disease. In some embodiments, the disease is a neoplastic disease. In some embodiments, the disease is a metabolic disease. In some embodiments, the disease is a lysosomal storage disease.

In some embodiments, the embodiment described herein is intended to be useful in the treatment of mitochondrial diseases or disorders. As used herein, “mitochondrial disease” refers to diseases caused by abnormal mitochondria, such as mitochondrial gene mutations, enzymatic pathways, etc. Examples of diseases include, but are not limited to, neurological diseases, loss of motor control, muscle weakness and pain, gastrointestinal diseases and dysphagia, poor growth, heart disease, liver disease, diabetes, respiratory complications, epilepsy, visual/hearing problems, lactic poisoning, slow development, and susceptibility to infection.

Examples of the diseases described in the present invention include, but are not limited to, genetic diseases, circulatory diseases, muscle diseases, diseases of the brain, central nervous system and immune system, Alzheimer's disease, secretase disorders, amyotrophic lateral sclerosis (ALS), autism, trinucleotide repeat amplification disorders, hearing diseases, gene targeted treatment of non-dividing cells (neurons, muscles), liver and kidney diseases, epithelial and pulmonary diseases, cancer, Ucher's syndrome or retinitis pigmentosa 39, cystic fibrosis, HIV and AIDS, beta thalassemia, sickle cell diseases, herpes simplex virus, autism, drug addiction, age-related macular degeneration, schizophrenia. Other diseases that are treated by correcting point mutations or introducing inactivation mutations into disease-related genes are known to those skilled in the art, and thus the present disclosure is not limited in this respect. In addition to the diseases exemplarily described in the present invention, other related diseases can also be treated by the strategy and prime editing system provided in the present invention, and such use is obvious to those skilled in the art.

Administration of the prime editing system or pharmaceutical composition of the present invention may be tailored to the weight and species of the patient or subject. The frequency of administration is within the allowable range for medicine or veterinary medicine. It depends on routine factors including the age, gender, general health, other conditions of the patient or subject, and the particular condition or symptom being addressed.

### VI. Kit

The present invention also includes a kit for use in the method of the present invention that comprises the components of the prime editing system of the present invention. The kit may also contain reagents for introducing the prime editing system into an organism or organism cell. Kits generally include labels indicating the intended use and/or method of use of the contents of the kit. The term label includes any written or recorded material provided on or with the kit or otherwise provided with the kit.

### EXAMPLES

### Example 1. PERV-RT Sequences Acquisition and Analysis

PERV sequences acquisition: PERV-A/C (Accession number: AY953542) and PERV-B (Accession number: AY099324) sequences were acquired through the NCBI database, and the positions and sequences of NC, RT and RNase H in the PERV sequence were analyzed using a conservative domain database to obtain PERV-AC-NC, PERV-B-NC and PERV-AC-RT (ΔRNase H) and PERV-B-RT (ΔRNase H).

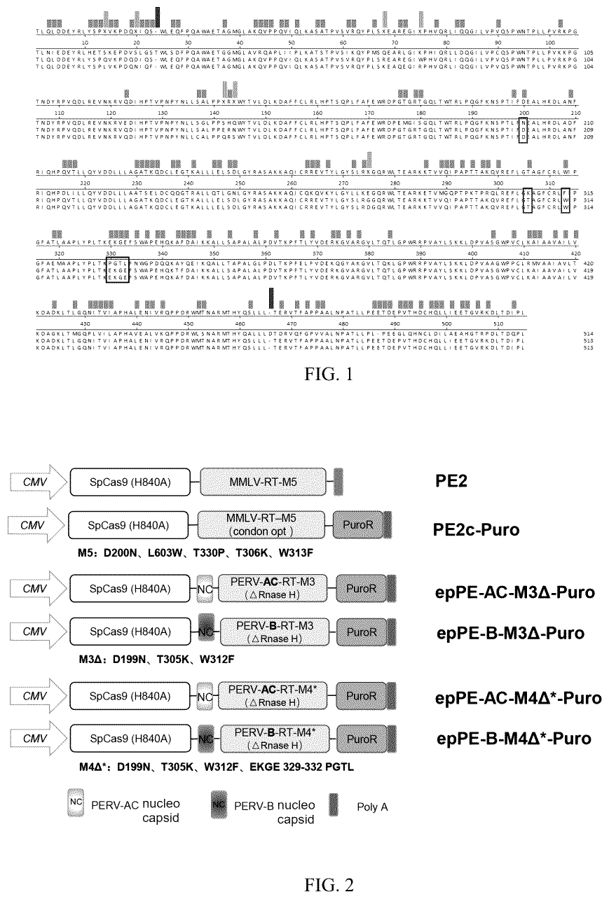

Sequence analysis: DNA sequences of MMLV-RT (ΔRNase H), PERV-AC-RT (ΔRNase H) and PERV-B-RT (ΔRNase H) were converted into protein sequences, and the differences of MMLV-RT (ΔRNase H), PERV-AC-RT (ΔRNase H) and PERV-B-RT (ΔRNase H) were compared using MegAlign (V 7.1.0) software (FIG. 1). It can be seen from the results that PERV-AC-RT and PERV-B-RT are highly similar, and there are only 11 amino acid differences; the similarity between PERV-RT and MMLV-RT is only 70%, and there are 153 and 150 amino acid differences between PERV-AC and PERV-B and MMLV-RT, respectively. It should be noted that among the four mutation sites of MMLV-RT in PE2, three are homologous to PERV-RT (FIG. 1, red box), and one site does not have homology, and the consecutive four bases at this site are different from PERV-RT (FIG. 1, black box).

### Example 2: Construction of Enhanced PERV-PE Editor (epPE)

### 2.1 Upgrading the PE2 Editor to Obtain PE2c-Puro

In order to enhance the editing efficiency of prime editing in mammalian cells, the present disclosure first upgraded the PE2 editor. A PE2 plasmid (No.: #132775) was first purchased from the Addgene plasmid sharing platform in the United States, and an online codon optimization tool (https://www.genscript.com.cn/codon-opt.html) was used to perform specific codon optimization on MMLV-RT in PE2 according to the porcine genome to obtain a MMLV-RT (condon opt) sequence, and Nanjing Jinsiruy Biotechnology Co., Ltd was entrusted to synthesize the sequence which was then used to replace the MMLV-RT sequence in the PE2 vector to obtain a PE2c vector. In addition, a 2A-Puro sequence was synthesized and inserted into the C-terminus of the RT structure of the PE2c vector, and the resulting vector was named PE2c-Puro (FIG. 2).

2.2 Construction of the epPE Editor
2.2.1 Construction of epPE-AC/B-m3-Puro

Using the online codon optimization tool (https://www.genscript.com.cn/codon-opt.html), for the PERV-AC-RT (ΔRNase H) and PERV-B-RT (ΔRNase H) sequences, specific codon optimization was performed according to the pig genome, and mutations were introduced corresponding to the 3 mutation sites (M3) homologous to the MMLV-RT in PE2 (D200N, T306K, W313F in MMLV-RT and the corresponding positions in the PERV-RT were D199N, T305K, W312F), to obtain the PERV-AC-RT-M3Δ and PERV-B-RT-M3Δ sequences, respectively. And the PERV-AC-RT-M3Δ and PERV-B-RT-M3Δ sequences were synthesized and used to replace MMLV-RT in PE2c-Puro by Nanjing Jinsey Biotechnology Co., Ltd., to obtain epPE-AC-M3Δ-Puro and epPE-B-M3Δ-Puro, respectively (FIG. 2).

2.2.2 Construction of epPE-AC/B-m4-Puro

The amino acids 329-332 of RT in epPE-AC-M3Δ-Puro and epPE-B-M3Δ-Puro were mutated from EKGE to PGTL (the DNA sequence was mutated from GAGAAGGGTGAA/GAGAAGGGAGAG to CCAGGCACCCTG) to obtain epPE-AC-M4Δ*-Puro and epPE-B-M4Δ*-Puro (FIG. 2).

### Example 3. Testing the Efficiency of Different epPEs in Immortalized BFP Fluorescence Reporter Porcine Fetal Fibroblast (PFF) Cells

3.1 Construction of pegRNA and ngRNA

pegRNA vector backbone pegRNA-GG (No.: 132777) and ngRNA vector backbone (No.: 107721) were purchased from the Addgene plasmid sharing platform in the United States, and the vector plasmid DNA was extracted using Takata MidiBest Kit (Takara, China) kit.

Vector digestion: 1× CutSmart buffer (Neb, USA), 1 μL of BsaI endonuclease (Neb, USA) and 2 μg pegRNA-GG or ngRNA empty plasmid DNA were contained in a 30 μL reaction system, and were digested for 2 hours at 37° C. and inactivated for 15 minutes at 80° C. The target fragment was separated from the digested product by 1.5% agarose gel, and purified by Axypre DNA Gel Extraction Kit (Axygen, USA).

The forward and reverse primers of peg RNA-gRNA, pegRNA 3′-extension, sgRNA scaffold and ngRNA were annealed into double strands: 25 μL reaction system contained 100 μM/L forward primer (F) and reverse primer (R), double distilled water and 1×T4 Ligation Buffer (Neb, USA), respectively; the EP tube containing the reaction system was sealed and placed in a beaker containing boiling water, and slowly cooled at room temperature.

Ligation of pegRNA: The ligation system was shown in Table 1, and after the system was prepared, the system was placed at room temperature for 10 minutes, then transferred to a PCR instrument for 16° C., 5 minutes; 37° C., 15 minutes (8 cycles); and finally inactivated at 80° C. for 15 minutes.

TABLE 1
pegRNA ligation system

| Components | Volume μL) |
|---|---|
| BsaI Linearized pU6-pegRNA-GG Vector | 30 ng |
| pegRNA-gRNA Annealed Product (2 μM) | 1 |
| pegRNA 3′-extension annealed product (2 μM) | 1 |
| sgRNA Scaffold Annealed Product (2 μM) | 1 |
| 10 × T4 DNA ligase buffer (NEB) | 1 |
| T4 DNA ligase (NEB) | 0.5 |
| BsaI-HFv2 (NEB) | 0.4 |
| Ultrapure Water | Up to 10 |

Ligation of ngRNA: 1 μL of annealed ngRNA was ligated in a 10 μL system at room temperature for 15 minutes under the action of Quick Ligase (Neb, USA), and then stored in a −20° C. refrigerator.

Preparation of High Purity Plasmid: 5 μL of the linked pegRNA/ngRNA was transformed into Trelief® 5α competent cells (Tsingke Biotech, China) according to the steps of the specification, and the transformed cells were plated on a solid LB (containing 10 g peptone, 5 g yeast extract 10 g NaCl, 15 g agar powder, and 30 mg ampicillin per liter of LB solid medium) plate, and cultured in an incubator at 37° C. overnight. 6 clones were picked for pegRNA/ngRNA respectively and cultured in 1 mL of liquid LB medium, 500 μL was taken and sent for Sanger sequencing (the sequencing primer was U6-F: atggactcatgcttaccgta), and the correctly linked clones were selected and added to 200 mL of liquid LB medium, and cultured on a shaker at 37° C. overnight, and a Takata MidiBest Kit (Takara, China) kit was used to extract high-purity and endotoxin-free plasmid DNA.

### 3.2 Cell Culture, Electro Transformation and Puromycin Screening

### 3.2.1 Cell Culture

The immortalized BFP fluorescence reporter PFF cell line was cultured in high glucose DMEM (Gibco, USA) medium containing 12% fetal bovine serum ((v/v), Excell, China) and 1% penicillin ((v/v), BI, China). The cell line was constructed by the laboratory, firstly, the SV40 large T antigen was site-directed knocked into the H11 safe harbor site of the primary PFFs of Bama pig, and then the BFP vector was site-directed integrated into the Rosa26 safe harbor site of immortalized PFF by the CRISPR Cas9 technology, and finally the BFP fluorescence reporter PFF cell line was obtained by serial passage.

### 3.2.2 Electro Transformation

When the cell confluence reached 85-90%, the cells were digested with 0.125% trypsin (Gibco, USA), and counted with a blood cell counting plate, 4×10⁵ cells were taken for each electro transformation reaction in a 15 mL centrifuge tube, centrifuged at 200×g for 5 minutes, 20 μL of Entranster-E electrotransformation solution (Engan, China), 1 μg of PE editor, 0.5 μg of pegRNA, and 0.5 μg of ngRNA were added after the supernatant was discarded, the cell suspension was transferred to a 16-well electric shock strip (Lonza, Germany) after mixing, and placed in an electric shock strip scaffold of a 4D-Nucleofector™ electrotransfer instrument, PFF cells were subjected to continuous electric shock twice using the CA137 program, and the electrotransformed cells were cultured in a 6-well cell culture dish. The culture medium was replaced 7 hours after electro transformation to remove dead cells caused by electric shock.

### 3.2.2 Puromycin Screening

2 μg/mL puromycin (Sigma, Japan) was added 48 hours after the electroporation for screening for 1 day, and then the culture medium was changed for further culture for 3 days.

### 3.3 Editing Efficiency Detection

After the cells were digested with 0.125% trypsin (Gibco, USA), the cells were collected into a 1.5 mL EP tube, centrifuged at 200×g for 5 minutes, the supernatant was discarded, 50 μL of 1×PBS buffer was added to resuspend the cells, and the cells were detected by using a flow cytometer (BD, USA), the wild-type fluorescence reporter cells were used as the background, and GFP-positive cells were circled by using two channels of FITC+PE, and the GFP ratio was the editing efficiency.

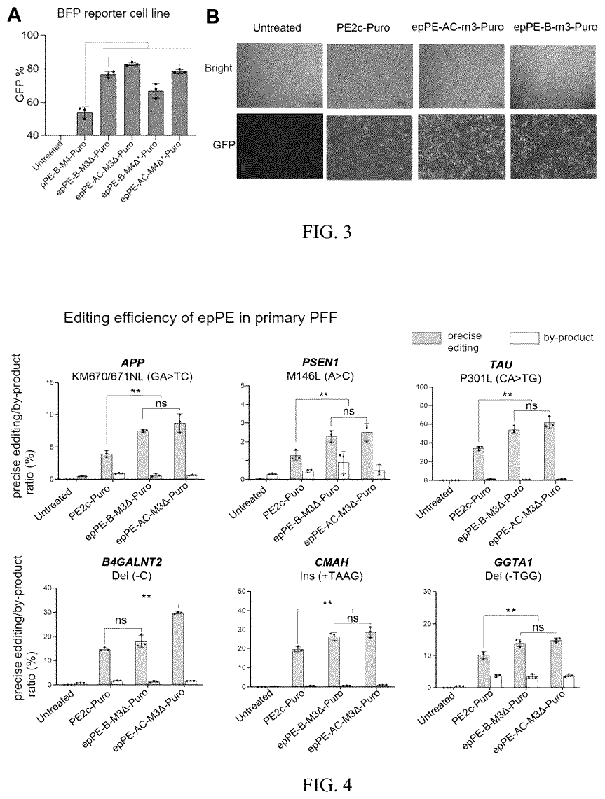

It was shown by flow cytometry that the efficiency of the four PERV-RT derived epPE editors was significantly higher (p<0.01) than that of the mouse RT PE2c-Puro editor, and the efficiency of epPE-AC-M3Δ-Puro was higher than that of epPE-B-M3Δ-Puro (p=0.007), and the efficiency of epPE-AC-M4Δ*-Puro was higher than that of epPE-B-M4Δ*-Puro (p=0.012). (FIG. 3)

### Example 4. Testing the Efficiency of AC- and B-Type PERV-RT epPE in Primary PFF Cells

### 4.1 Acquisition of Primary PFF Cells

The sows for 31 days gestation were sacrificed and dissected, the whole uterus was taken out, the fetus and the fetal membrane were taken out one by one, and transferred into a DMEM solution containing 5% (v/v) penicillin and streptomycin (v/v; Gibco: 15140-122) for storage in a refrigerator at 4° C. In a clean bench, the isolated fetus was transferred one by one to a sterile 10 cm culture dish, and the head, viscera, limbs and tail of the fetus were removed with sterile forceps and scissors; the remaining part of the fetus was cleaned with a PBS solution containing 5% (v/v) penicillin and streptomycin (Gibco: 15140-122)), and then the remaining part of the fetus was cut to about 1 mm³ within 10-15 minutes, and 5 ml of collagenase working solution was added to the cut tissue; the tip of 1 ml of sterile pipettor was cut off with scissors, and the cut tissue pieces were transferred to a 15 ml sterile centrifuge tube, sealed and placed in a shaker at 37° C. for incubation for 2 hours. After that, the centrifuge tube was centrifuged at 1000 rpm for 5 minutes, the upper collagenase working solution was removed, and the incubated tissues were evenly transferred into two T75 culture dishes and cultured in a 37° C., 5% CO₂ sterile incubator. The culture was continued to the next morning (note: after about 20 hours of cell culture, if the color of the cell culture medium became yellow, it was needed to be replaced with a new culture medium), and the adherence of the cells and the separation from the cell tissues were observed under a microscope, and the construction of the porcine primary PFF cell line was completed when the cell proliferation and growth confluency reached more than 85%, and the cells were cryopreserved or subcultured.

### 4.2 Primary PFF Cell Culture, Electrotransformation and Puromycin Screening

Cells were cultured in 12% (v/v) fetal bovine serum (Excell, China) and 1% (v/v) penicillin-streptomycin (BI, China) high-glucose DMEM (Gibco, USA) medium at 37° C. in a 5% carbon dioxide incubator. When the cell confluence reached 85-90%, 0.125% trypsin (Gibco, USA) was used to digest the cells, and a blood cell counting plate was used for counting, 4×10⁵ cells were taken in a 15 mL centrifuge tube for each electrotransformation reaction, centrifuged at 200×g for 5 minutes, 20 μL of Entranster-E electrotransformation solution (Engn, China), 1 μg of PE editor, 0.5 μg of pegRNA, and 0.5 μg of ngRNA were added after the supernatant was discarded, the cell suspension was transferred to a 16-well electric shock strip (Lonza, Germany) after mixing, the cell suspension was placed in an electric shock strip scaffold of a 4 D-Nucleofector™ electrotransfer instrument, and electric shock was continuously performed twice using the CA-137 program, and the electrotransformed cells were cultured in a 6-well cell culture dish. The culture medium was replaced 7 hours after electrorotation to remove dead cells caused by electric shock. 2 μg/mL puromycin (Sigma, Japan) was added 48 hours after the electroporation for screening for 1 day, and then the culture medium was changed for further culture for 3 days.

### 4.3 Editing Efficiency Detection

### 4.3.1 Cell DNA Extraction

Cells were digested with 0.125% trypsin and collected into 200 μL EP tubes, centrifuged at 750×g for 5 minutes, and the supernatant was removed using a vacuum pipette. 12 μL of cell lysate (0.45% NP40 lysate (Gibco, USA), 6% protease K (Sigma, Japan) were added to resuspend the cells, and the cells were placed in a PCR instrument, and the reaction procedure was: 1.5 hours at 56° C., and 15 minutes at 85° C.

### 4.3.2 Amplification of the Target Site

Two rounds of PCR amplification were performed on the target site using the corresponding primers, wherein the first round of PCR amplified the target site and the second round of PCR labeled the sample using the tagged primers. Both rounds of PCR were amplified using PrimeSTAR Max DNA Polymerase (Takara, Japan). Each reaction contained 8 μL of PrimeSTAR Max DNA Polymerase buffer, 1 μL of forward and reverse primers, 1 μL of DNA template, and 5 μL of ultrapure water. PCR reaction conditions: 94 for 5 minutes; (94 for 30 seconds, 68° C. (0.5° C. lower per cycle) for 30 seconds, 72 for 1 minute) for 26 cycles; (94 for 30 seconds, 55 for 30 seconds, 72 for 1 minute) for 14 cycles; 72 for 5 minutes.

4.3.3 The samples was mixed and purified by gel cutting. The amplification products with different tags were mixed in a manner of 5 μL of each sample and 8-12 samples as a group. After the loading buffer was added to the mixed sample, the mixed sample was separated by electrophoresis using a 2% agarose gel at a voltage of 180V for 20 minutes, and then the target fragment band was cut off on an ultraviolet gel cutting table, and purified and recovered using an Axypre DNA Gel Extraction Kit (Axygen, USA) according to the method instructed by the specification, and finally DNA was dissolved using ultrapure water.
4.3.4 High throughput sequencing. 2 μg of purified PCR product was submitted to Beijing Novo-Genet Technology Co., Ltd., and sequenced. DNA was first A-tailed using the Next® Ultra™ DNA Library Prep Kit for Illumina kit and ligated to Illumina sequencing adapters. The DNA was then purified using AMPure XP system and quantified, diluted using Qubit 3.0. The insertion fragments of the library were detected using Agilent 2100, and the effective concentration thereof was accurately quantified using the Q-PCR method for libraries whose insertion fragment size met expectations. After that, the PE150 platform of NovaSeq 6000 of Illumina high-throughput sequencer was used for paired-end sequencing.

### 4.3.5 Editing Efficiency Analysis

First, Fasstp (V 0.19.7) software was used to filter the sequences with the base quality value Qphred≤5 in the sequencing data and the reads where the content of N in the read exceeding 10% of the base number of the read to obtain Clean Data. The mixed sample data was split using fastq-multx (V 1.31) software and each sample would yield R1.fastq and R2.fastq. The editing efficiency was analyzed using CRISPR esso2 (V 2.0.43) software, each sample provided parameters such as ref_seq, pegRNA_spacer_seq, pegRNA_extension_pegRNA_scaffold_seq, and nicking_guide_seq, with default values for the other parameters. ref_seq was a reference sequence for PCR amplification, and the pegRNA_extension_pegRNA_scaffold_seq sequence was a complete sgRNA scaffold sequence. For the final result, Prime-edited UNMODIFILED was considered as an accurate edit, and other edit types were considered as unwanted by-products.

The results showed that the average editing efficiency of the PERV-RT-derived epPE editor was higher than that of the murine RT PE2c-Puro editor at 6 editing sites tested in the primary PFF, and the average efficiency of epPE-AC-M3Δ-Puro was higher than that of epPE-B-M3Δ-Puro (FIG. 4).

### Example 5 Testing the Efficiency of epPE in Other Cells

### 5.1 Cell Culture

Canine fetal fibroblasts (DFF) were isolated from a female canine fetus on 30 days of gestation, and the specific establishment method was the same as that of PFF; goat fetal fibroblasts (SFF) were given by Yunnan Agriculture University Weijiang Laboratory, and the cells were isolated from a small tailed han sheep fetus; 293T cells were purchased from the Cell Bank of the Type Culture Collection Committee of Chinese Academy. All three cells were cultured in high-glucose DMEM (Gibco, USA) culture medium containing 12% (v/v) fetal bovine serum (Excell, China) and 1% (v/v) penicillin-streptomycin (BI, China) at 37° C. in a 5% CO₂ incubator.

### 5.2 Cell Transfection

DFF and SFF: The method is similar to the transfection method of PFF, and the difference lies in that when DFF and SFF were transformed, the cells were shocked by using the EN-150 program, then again by using the CA-113 program.

293T cells: cells were inoculated into a 24-well culture dish 1-2 days before cell transfection, and when the cell confluency reached 50-60%, the cells were transfected with Lipofectamine 3000 reagent (Invitrogen, USA), and each transfection reaction included 1 μg PE editor, 0.5 μg pegRNA, 0.5 μg ngRNA and 1 μL Lipofectamine 3000 reagent.

### 5.3 Puromycin Screening

SFF and DFF cells: 3 μg/mL puromycin (Sigma, Japan) was added 48 hours after electrotransformation for screening for 2 days, and then the culture medium was changed for further culture for 2 days.

293T cells: after transfection, 3 μg/mL puromycin (Sigma, Japan) was added for screening for 3 days, and then the culture medium was changed and cultured for another 2 days.

### 5.4 Editing Efficiency Detection

The method is same with the detection of editing efficiency of PFF.

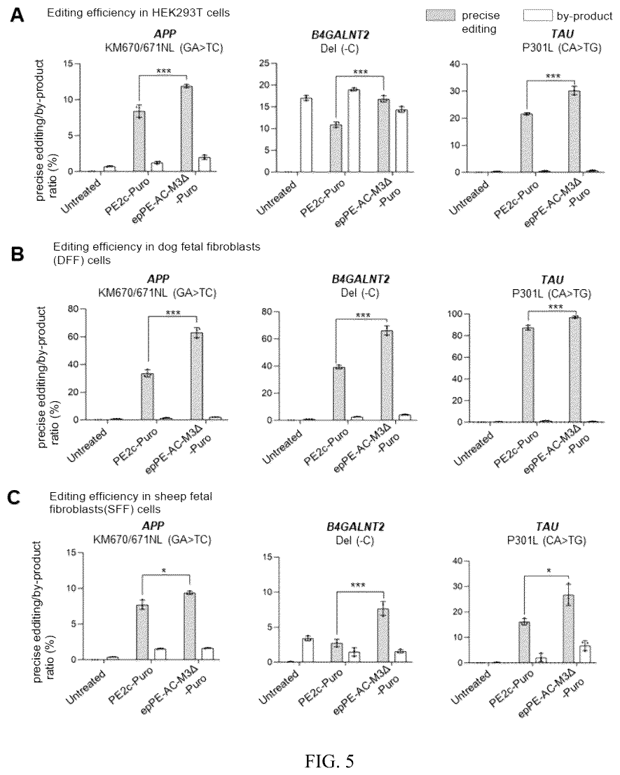

After analysis, as shown in FIG. 5, the efficiency of the epPE editor in other mammals such as SFF, DFF and 293T is higher than that of the PE2c-Puro vector of murine MMLV-RT.

### Example 6. Use of PERV RT Isolated from Bama Pig in Prime Editing

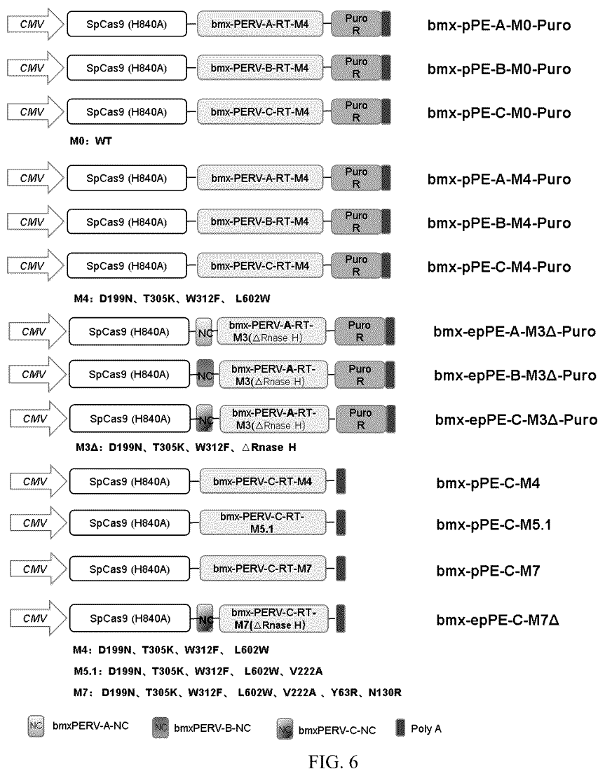

The inventors further isolated 3 types of PERV-RT (bmx-PERV-A-RT-M0, bmx-PERV-B-RT-M0 and bmx-PERV-C-RT-M0) from the Bama pig DNA by PCR amplification, which were used to respectively replace MMLV-RT in the PE2c-Puro vector to obtain pPE editors of three PERV-RTs, A, B and C, respectively, and PE editors carrying different mutation sites were constructed for bmx-PERV-RT: M0 represents wild type and does not contain mutations; M4 represents four mutations (D199N, T305K, W312F and L602W), as shown in FIG. 6. The PERV-RTs and the corresponding primer editors derived from Bama Swine were all labeled with “bmx-” to distinguish from the PERV-RT and the corresponding primer editors from NCBI.

The editing activities of different editors in different cell lines were tested as described above.

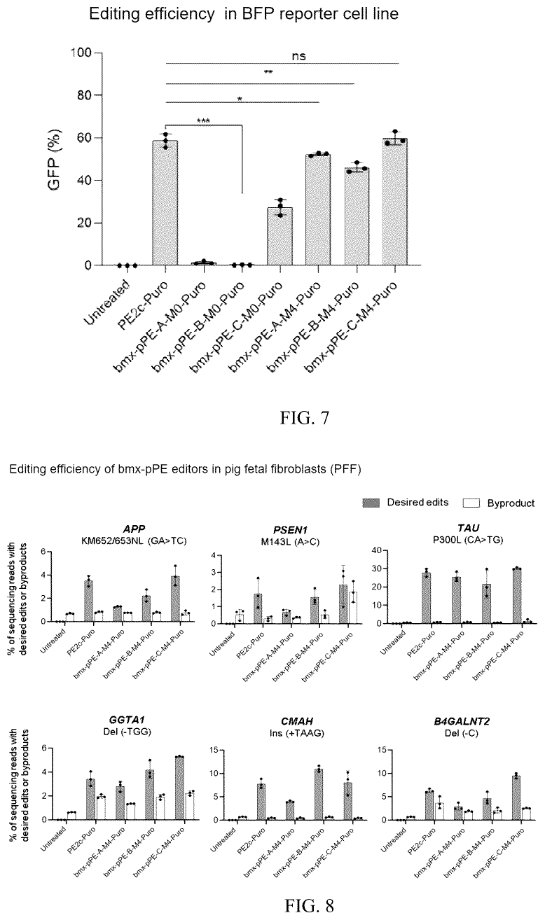

FIG. 7 shows that in the BFP reporter cell line, the pPE editor containing Bama pig Type C PERV reverse transcriptase has higher editing activity, and after introducing the mutations, all three PERV-RTs can obtain higher editing activity.

FIG. 8 shows the editing efficiency of the pPE editors of Bama Pig PERV reverse transcriptase containing 4 point mutations in PFF, and bmx-pPE-C-M4-puro has overall higher efficiency.

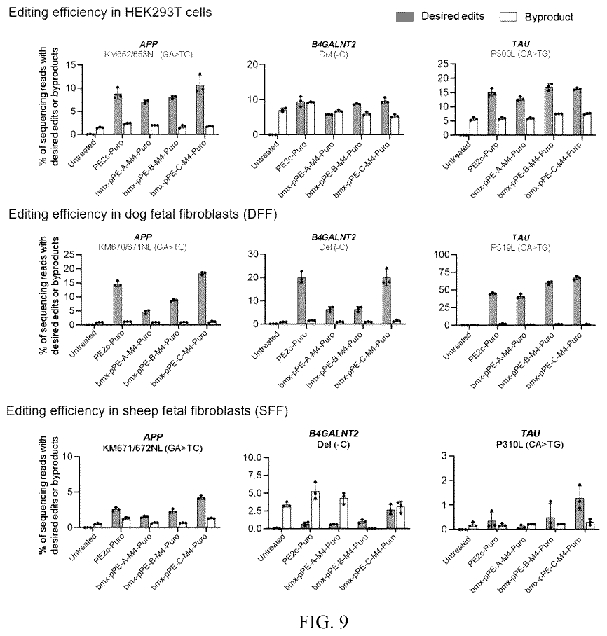

FIG. 9 shows the editing efficiency of the pPE editors of Bama pig PERV reverse transcriptase containing 4 point mutations in HEK293T, canine fetal fibroblasts and goat fetal fibroblasts, and bmx-pPE-C-M4-puro exhibits higher efficiency at most sites.

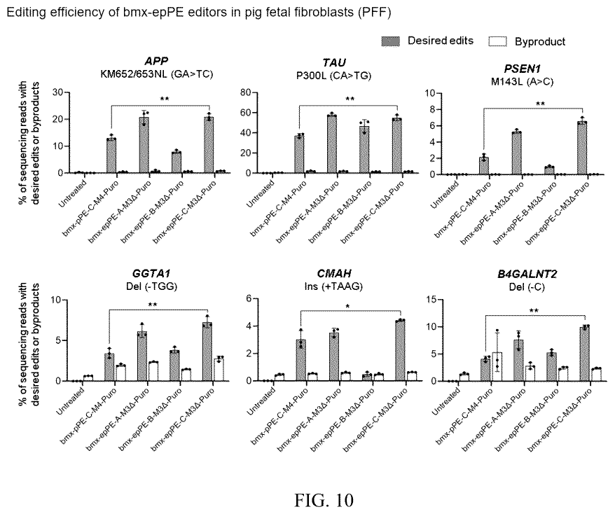

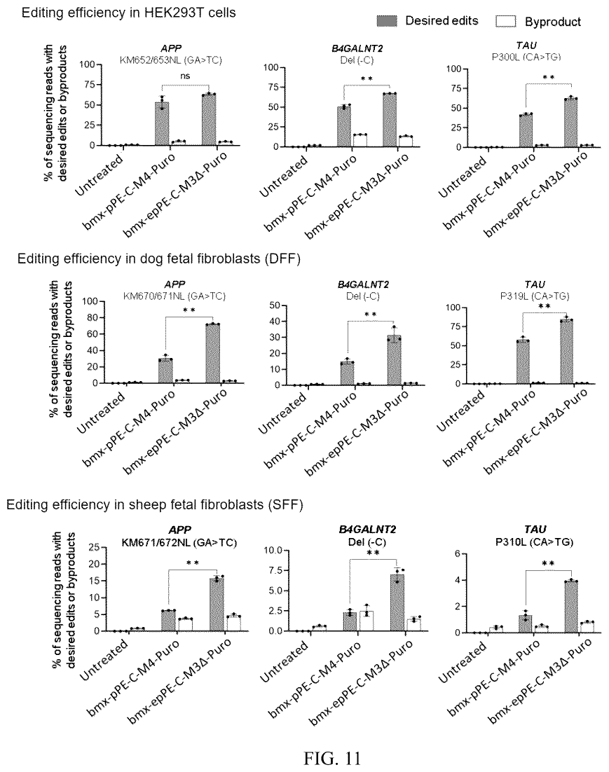

FIG. 10 shows the editing efficiency of the epPE editors of Bama pig PERV reverse transcriptase containing 4 point mutations in PFF, and compared with the bmx-epPE of type A and type B, the bmx-epPE editor of type C exhibits overall higher efficiency which is significantly higher than that of bmx-pPE.

FIG. 11 shows the editing efficiency of bmx-epPE editor C (bmx-epPE-C-M3Δ-Puro) in HEK293T, canine fetal fibroblasts and goat fetal fibroblasts, and the overall editing efficiency of bmx-epPE-C-M3Δ-Puro in three cell lines is significantly higher than that of bmx-pPE-C-M4-Puro.

### Example 7. The Bama Pig PERV-RT Prime Editing System Carrying 7 Point Mutations has Higher Efficiency

On the basis that bmx-PERV-RT of type C has higher editing efficiency in the prime editing system, the inventors performed a large number of mutation site screening on bmx-PERV-RT of type C in 293T cells.

### 7.1 Selection of Mutation Sites

7.1.1 Mutation Sites from the MMLV-RT Prime Editing System which have been Reported and have Homology with PERV-RT.

According to the reports of Anzalone et al. Nature (2019), Ni et al. Genome Biology (2023), six potential favorable sites of D199N, T305K, W312F, E329P, L602W and V222A were obtained by selecting sites that are effective in the MMLV-RT prime editing system and have homology with PERV-RT.

7.1.2 Potential Favorable Mutation Sites were Obtained by Molecular Docking.

Mutating polar negatively charged amino acids (D, E) and polar uncharged amino acids (S, T, C, N, Q, Y, G) that bind to DNA or RNA in RT into polar positively charged amino acids (R, K) may enhance the interaction between RT and DNA/RNA, thereby improving the prime editing efficiency. To this end, the present inventors used Phyre2 software to obtain the predicted protein structure of the bmx-PERV-C-RT-M5.1 variant, downloaded the PDB file of the MMLV-RT-DNA-RNA complex: 4KHQ from the PDB number database, and used PyMOL software to align the bmx-PERV-C-RT-M5.1 variant with 4KHQ to find out the amino acids of the interaction between bmx-PERV-C-RT-M5.1 and DNA/RNA, and selected all polar negative charged amino acid and polarity uncharged amino acids to be mutated into R, to obtain a total of 11 mutation sites: Y43R, Y108R, Q112R, D113R, E116R, D149R, D152R, G190R, N118R, N130R, G269R, T305R, Y324R.

7.1.3 Obtaining Potential Favorable Sites from Literature Reports
7.1.3.1 7 potential mutation sites: S86R, Q112R, Q189F, Q220R, T286A, E332Q, and I434R were obtained in total by selecting mutation sites that have been reported to improve MMLV-RT reverse transcription performance but have not been verified in the PE system and have homology with PERV-RT.
7.1.3.2 Doman et al. Cell (2023) used phage assisted evolution system to obtain a large number of mutation sites, but the efficiency of these mutation sites has not been verified in PE system, and the present inventors screened out 9 high frequency and homology mutation sites with PERV-RT: T127N, P131S, P195S, K232N, E338N, C408R, D423V, M4561, T4571.

### 7.2 293 T Cell Fluorescence Reporter Gene Co-Transfection Experiment

1×10⁵ cells were inoculated into a 24-well culture dish 17-24 hours before cell transfection, and when the cell confluence reached 50-80%, transfection was performed using H4000 reagent (Ingran, China), and each transfection reaction included 200 ng PE editor, 100 ng pegRNA, 100 ng ngRNA, 10 ng fluorescent reporter gene vector and 0.5 μL H4000 reagent. The cells were collected 48 hours after transfection, and detected by flow cytometry (BD, USA), with wild-type fluorescence reporter cells as background, and GFP positive cells were circled with FITC+PE double channel, with GFP ratio as editing efficiency.

### 7.3 293 T-Cell Endogenous Editing Site Editing Experiment

1×10⁵ cells were inoculated into a 24-well culture dish 17-24 h before cell transfection, and when the cell confluency reached 50-80%, transfection was performed using H4000 reagent (Ingran, China), and each transfection reaction included 400 ng PE editor, 200 ng pegRNA, 200 ng ngRNA and 1 μL H4000 reagent. The culture medium was changed 24 hours after transfection, and the cells were collected at about 72 hours after transfection, and the editing efficiency was detected with reference to the method of 4.3.

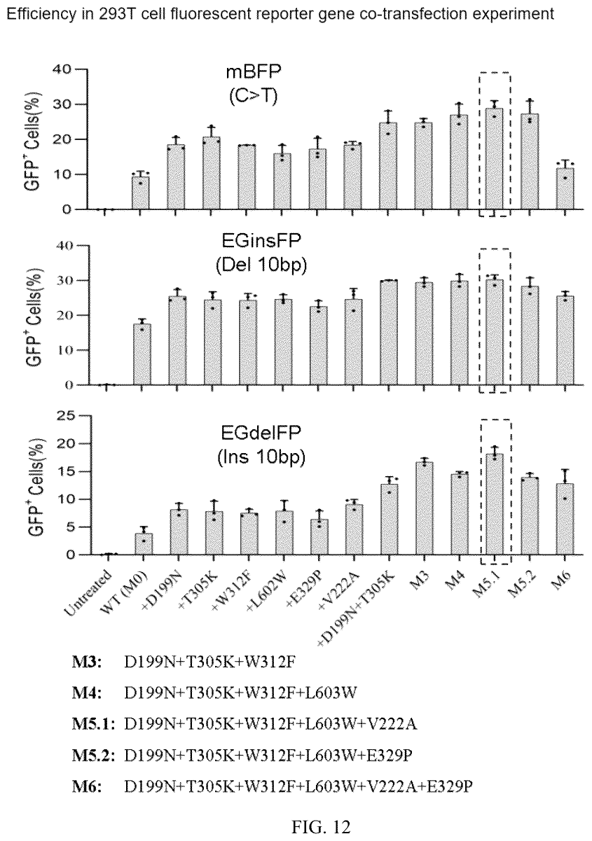

FIG. 12 shows the efficiency of Bmx-PERV-RT carrying 6 mutation sites and combinations thereof reported in the MMLV-RT prime editing system and having homology with PERV-RT in the co-transfection experiment of 293T cell fluorescent reporter gene, and the data show that the PERV-RT variant carrying 5 amino acid mutations (D199N, T305K, W312F, L602W, V222A) (named as M5.1) exhibits the overall optimal efficiency in 3 editing forms.

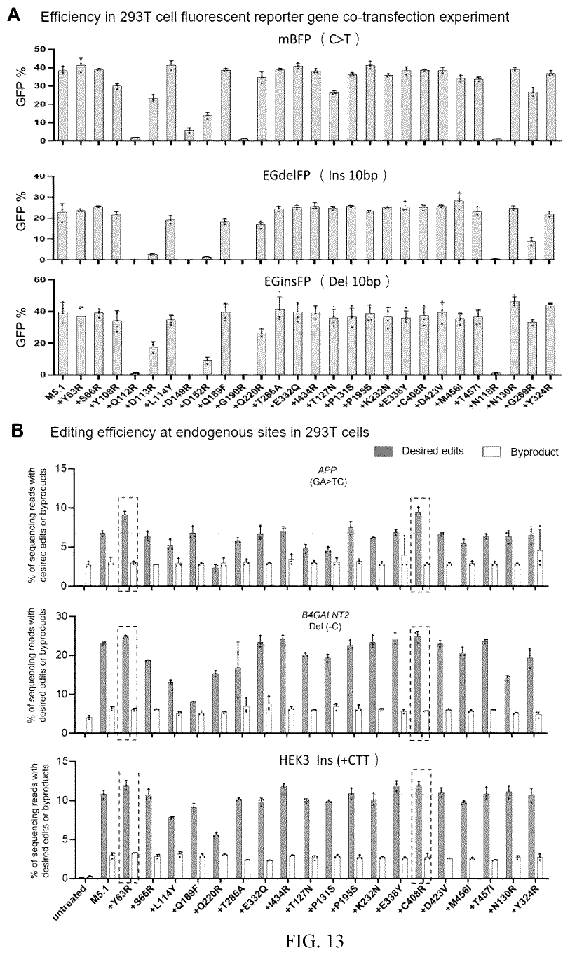

FIG. 13 shows the efficiency of the M5.1-based pPE editor with additional new potential favorable mutation sites in 293T cells. FIG. 13A shows the efficiency of a total of 27 new potential mutation sites in 293T cell fluorescence reporter gene co-transfection experiments obtained using molecular docking and reported in the literature, of which 8 sites significantly reduced PE editing efficiency. FIG. 13B shows the efficiency of the remaining 19 mutation sites at the endogenous sites in 293T cells, wherein Y63R and C408R showed different degrees of improvement in editing efficiency.

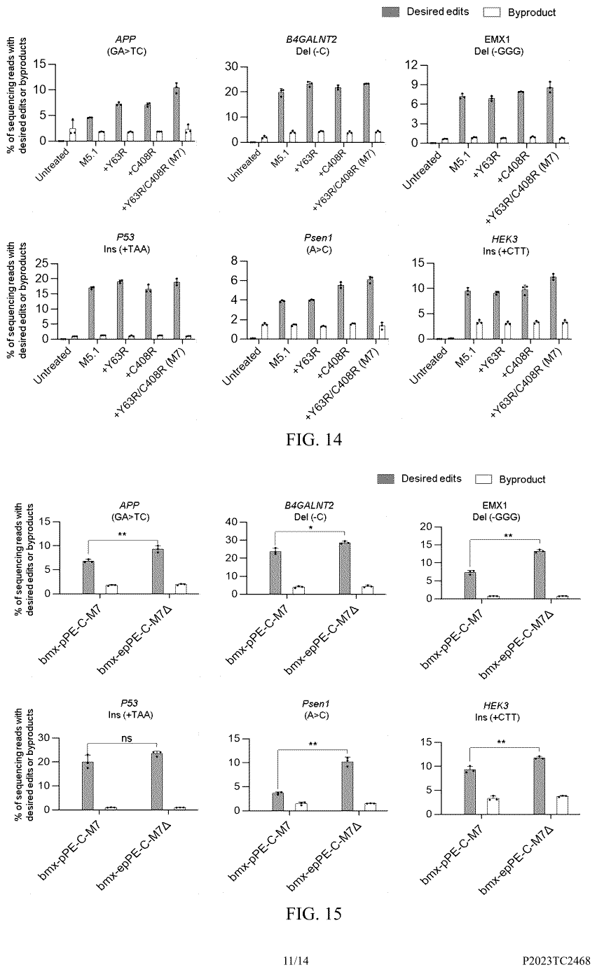

FIG. 14 shows the editing efficiency of M5.1, M5.1+Y63R, M5.1+C408R and its combination M5.1+Y63R+C408R (M7) at the endogenous sites in 293T cells, and the results showed that M7 has the synergistic editing efficiency at multiple sites, and the efficiency at all sites is significantly better than that of M5.1.

FIG. 15 shows the editing efficiency of the epPE editor based on M7 in 293T cells, and the efficiency of bmx-epPE-C-M7A with additional 7 point mutations is further improved compared with bmx-pPE-C-M7.

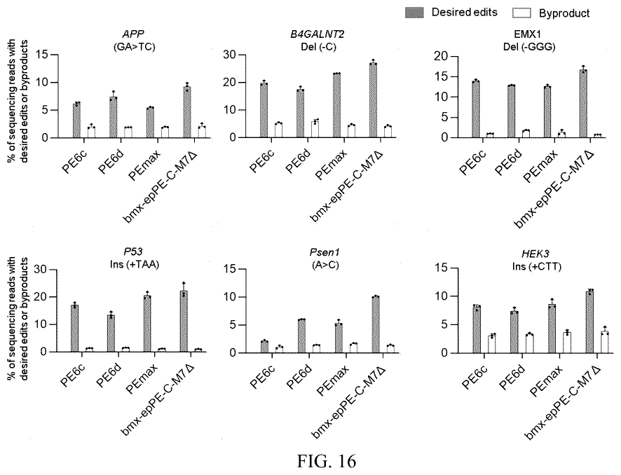

FIG. 16 shows the efficiency comparison between bmx-epPE-C-M7A and the current known PEmax, PE6c, PE6d editors with optimal efficiency in 293T cells. The results show that the overall efficiency of bmx-epPE-C-M7A is higher than that of PEmax, PE6c and PE6d.

### Example 8. tCsy4-pegRNA has Higher Editing Efficiency

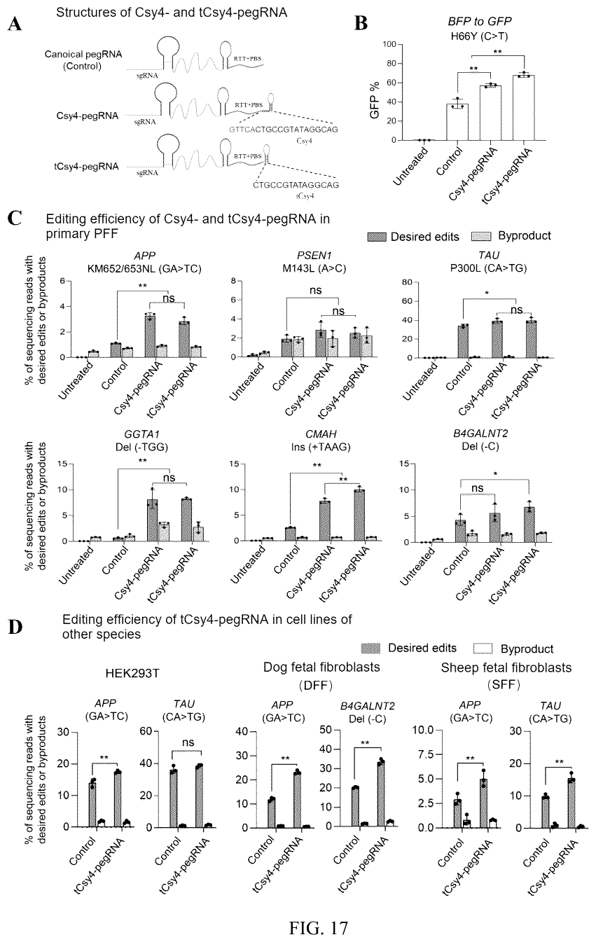

The 3′ end of the conventional pegRNA is a naked RNA, which is easily degraded in cells, thereby affecting the efficiency of the pegRNA. In addition, since PBS is typically 10-16 nt at the 3′ end of the pegRNA and complementary to the partial spacer at the 5′ end of the pegRNA, their annealing is expected to result in pegRNA circularization, which may hinder editing. It has been reported that by adding the Csy4 recognition sequence (hairpin structure) to the end of the pegRNA, the efficiency of PE in HKE293T, HeLa and Neuro-2a cells is significantly improved. The inventors also designed a truncated Csy4 (tCsy4, SEQ ID NO: 51). The editing efficiency of tCsy4-pegRNA was compared with Csy4-pegRNA and conventional pegRNA (FIG. 17A). The results showed that the editing efficiency of Csy4-pegRNA (5 of 7 sites) and tCsy4-pegRNA (6 of 7 sites) was significantly higher than that of conventional pegRNA, and the editing efficiency of tCsy4-pegRNA was similar to or higher than that of Csy4-pegRNA (FIG. 17B, C). In addition, the efficiency of tCsy4-pegRNA in HEK293T, DFF and SFF was verified, and the data showed that tCsy4-pegRNA could further improve the PE efficiency in three cell lines (FIG. 17D).

### Example 9. Small Molecules can Significantly Improve the Efficiency of Prime Editing in Mammalian Cells

It is known that some small molecule compounds can significantly improve the genome editing efficiency of CRISPR/Cas9. From this, it can be expected that some small molecules may also improve the editing efficiency of PE. Nine small molecules that have been demonstrated to be effective in the CRISPR/Cas9 system were first tested in the fluorescent reporter PFF cell line: 10 μM Scr7, 2 μM Nu7441, 25 μM L189, 40 μM L755507, 5 μM Yu238259, 0.1 μM Brefeldin A, 10 nM TSA, 20 μM RS-1, 200 ng/mL Nocodazole, 25 μM L189. Cells were treated by adding the corresponding concentrations of small molecules or combinations of small molecules, respectively, to the medium after transfection.

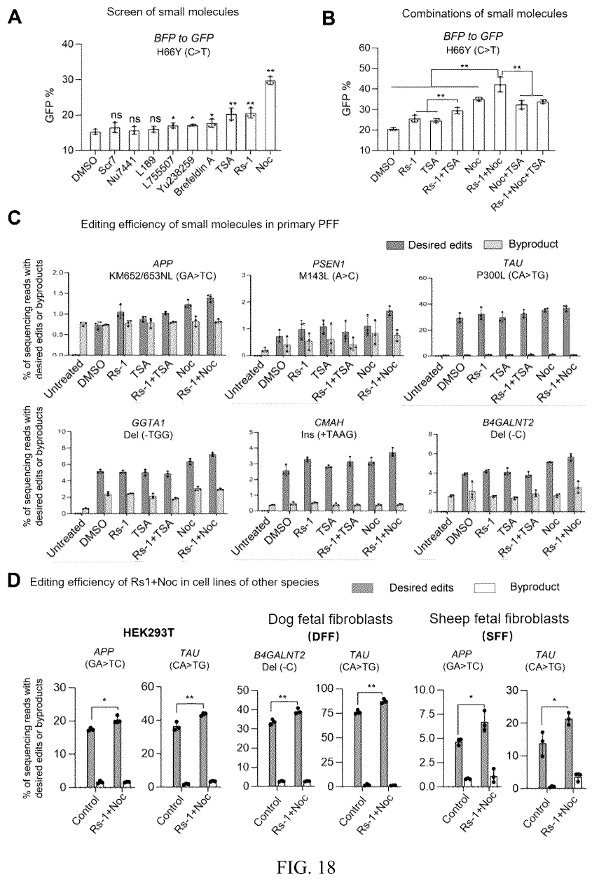

The data shows that L755507, BrefeldinA, TSA, Rs-1, and Nocodazole significantly (P<0.05 or P<0.01) improved PE efficiency, with TSA, Rs-1, and Nocodazole being more efficient, increasing editing efficiency by 34.4%, 32.7%, and 66.9% (FIG. 18A). The combination of Rs-1+TSA or Rs-1+Nocodazole significantly improved (P<0.01) editing efficiency compared to using a single molecule. The editing efficiency of the Rs-1+Nocodazole group was the highest, and the efficiency was improved by 106.0% compared with the DMSO group (FIG. 18B). The effect of Rs-1, Nocodazole, TSA, and the combination of Rs-1+TSA and Rs-1+Nocodazole in primary PFFs was then verified. The data show that in primary PFF using Rs-1, TSA, and Nocodazole, the overall editing efficiency for the 6 sites was improved by 20.8%, 14.3%, and 36.4%, respectively. The Rs-1+Nocodazole combination improved the editing efficiency by 60.54%, while the Rs-1+TSA combination showed no additive effect (FIG. 18C). In addition, the efficiency of the Rs-1+Nocodazole combination in HEK293T, DFF and SFF was verified, and the data showed that the Rs-1+Nocodazole combination can further improve the PE efficiency in three cell lines (FIG. 18D).

### Information of the Small Molecule Compounds Involved in the Present Invention

### RS-1:

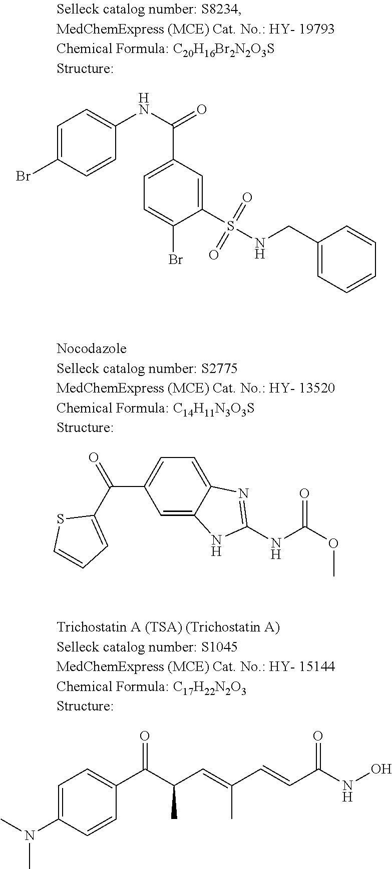

### Sequence Information Related to the Present Invention

EQ ID NO: 1 PERV-AC-RT-M0
TLQLDDEYRLYSPQVKPDQDIQSWLEQFPQAWAETAGMGLAKQVPPQVIQLKASATPVSVRQYPLSREARE
GIWPHVQRLIQQGILVPVQSPWNTPLLPVRKPGTNDYRPVQDLREVNKRVQDIHPTVPNPYNLLSALPPER
NWYTVLDLKDAFFCLRLHPTSQPLFAFEWRDPGTGRTGQLTWTRLPQGFKNSPTIFDEALHRDLANFRIQH
PQVTLLQYVDDLLLAGATKQDCLEGTKALLLELSDLGYRASAKKAQICRREVTYLGYSLRGGQRWLTEARK
KTVVQIPAPTTAKQVREFLGTAGFCRLWIPGFATLAAPLYPLTKEKGEFSWAPEHQKTEDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLLTERVTFAPPAALNPATLLPEETDEPVTHDCHQL
LIEETGVRKDLTDIPLTGEVLTWFTDGSSYVVEGKRMAGAAVVDGTHTIWASSLPEGTSAQKAELMALTQA
LRLAEGKSINIYTDSRYAFATAHVHGAIYKQRGLLTSAGREIKNKEEILSLLEALHLPKRLAIIHCPGHQK
AKDLISRGNQMADRVAKQAAQAVNLLPIIETPKAPEPRRQ
Note: underlined is the RNase H domain
SEQ ID NO: 2 PERV-B-RT-M0
TLQLDDEYRLYSPLVKPDQNIQFWLEQFPQAWAETAGMGLAKQVPPQVIQLKASATPVSVRQYPLSKEAQE
GIRPHVQRLIQQGILVPVQSPWNTPLLPVRKPGTNDYRPVQDLREVNKRVQDIHPTVPNPYNLLCALPPQR
SWYTVLDLKDAFFCLRLHPTSQPLFAFEWRDPGTGRTGQLTWTRLPQGFKNSPTIFDEALHRDLANFRIQH
PQVTLLQYVDDLLLAGATKQDCLEGTKALLLELSDLGYRASAKKAQICRREVTYLGYSLRDGQRWLTEARK
KTVVQIPAPTTAKQVREFLGTAGFCRLWIPGFATLAAPLYPLTKEKGEFSWAPEHQKAFDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLLTERVTFAPPAALNPATLLPEETDEPVTHDCHQL
LIEETGVRKDLTDIPLTGEVLTWFTDGSSYVVEGKRMAGAAVVDGTRTIWASSLPEGTSAQKAELMALTQA
LRLAEGKSINIYTDSRYAFATAHVHGAIYKQRGLLTSAGREIKNKEEILSLLEALHLPKRLAIIHCPGHQK
AKDPISRGNQMADRVAKQAAQGVNLLPMIETPKAPEPGRQ
Note: underlined is the RNase H domain
SEQ ID NO: 3 bmx-PERV-A-RT-M0
TLQLDDEYRLYSPLVKPDQNIQFWLEQFPQAWAETAGMGLAKQVPPQVIQLKASATPVSVRQYPLSKEARE
GIRSHVQRLIQQGILVPVQSPWNTPLLPVRKPGTNDYRPVQDLREVNKRVQDIHPTVPNPYNLLCALPPQR
SWYTVLDLKDAFFCLRLHPTSQPLFAFEWRDPGAGRTGQLTWTRLPQGFKNSPTIFDEALHRDLANFRIQH
PQVTLLQYVDDLLLAGATKQDCLEGTKALLLELSDLGYRASAKKAQICRREVTYLGYSLRGGQRWLMEARK
KTVVQIPAPTTAKQVREFLGTAGFCRLWIPGFATLAAPLYPLTKEKGEFSWAPEHQKAFDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLLTERVTFAPPAALNPATLLPEEADEPVTHDCHQL
LIEETGVRKDLTDIPLTGEVLTWFTDRSSYVVEGKRMAGAAVVDGTHTIWASSLPEGTSAQKAELMALTQA
LRLAEGKSINIYTDSRYAFATAHVHRAIYKQRGLLTSAGREIKNKEEILSLLEALHLPKRLAIIHCPGHQK
AKDPISRGNQMADRVTKQAAQGVNLLPIIETPKAPEP
Note: underlined is the RNase H domain
SEQ ID NO: 4 bmx-PERV-B-RT-M0
TLQLDDEYRLYSPQVKPDQDIQSWLEQFPQAWAETAGMGLAKQVPPQVIQLKASATPVSVRQYPLSREARE
GIWPHVQRLIQQGILVPVQSPWNTPLLPVRKPGTNDYRPVQDLREVNKRVQDIHPTVPNPYNLLSALPPEQ
NWYTVLDLKDAFFCLRLHPTSQPLFAFKWRDPGTGRTGQLTWTRLPQGFKNSPTIFDEALHRDLANFRIQH
PQVTLLQYVDDLLLAGATKQDCLEGTKALLLELSDLGYRASAKKAQICRREVTYLGYSLRGGQRWLTEARK
KTVVQIPAPTTAKQVREFLGTAGFCRLWIPGFATLAAPLYLLTKEKGEFSWAPEHQKAFDAIKKSLLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNACMTHYQSLLLTERVTFAPPAALNPATLLPEETDEPVTHDCHQL
LIEETGVRKDLTDIPLTGEVLTWFTDGSSYVVEGKRMAGAAVVDGTRTIWASSLPEGTSAQKAELMALTQA
LRLAEGKSINIYTDSRYAFATAHVHGAIYKQRGLLTSAGREIKNKEEILSLLEALHLPKRLAIIHCPGHQK
AKDLISRGNQMADRVAKQAAQAVNLLPIIETPKAPEP
Note: underlined is the RNase H domain
SEQ ID NO: 5 bmx-PERV-C-RT-M0
TLQLDDEYRLYSPLVKPDQNIQFWLEQFPQAWAETAGVGLAKQVPPQVIQLKASATPVSVRQYPLSKEARE
GIRPHVQRLIQQGILVPVQSPWNTPLLPVRKPGTNDYRPVQDLREVNKRVQDIHPTVPNPYNLLCALPPQR
SWYTVLDLKDAFFCLRLHPTSQPLFAFEWRDPGAGRTGQLTWTRLPQGFKNSPTIFDEALHRDLANFRIQH
PQVTLLQYVDDLLLAGATKQDCLEGTKALLLELSDLGYRASAKKAQICRREVTYLGYSLRGGQRWLTEARK
RTVVQIPAPTTAKQVREFLGTAGFCRLWIPGFATLAAPLYPLTKEKGEFSWAPEHQKAFDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLLTERVTFAPPAALNPATLLPEETDEPVTHDCHQL
LIEETGVRKDLTDIPLTGEVLTWFTDGSSYVVEGKRMAGAAVVDGTRTIWASSLPEGTSAQKAELMALTQA
LRLAEGKSINIYTDSRYAFATAHVHGAIYKQRGLLTSAGREIKNKEEILSLLEALHLPKRLAIIHCPGHQK
AKDPISRGNQMADRVAKQAAQGVNLLPIIETPKAPEP
Note: underlined is the RNase H domain
SEQ ID NO: 6 AC-RT-M0Δ
TLQLDDEYRLYSPLVKPDQNIQFWLEQFPQAWAETAGMGLAKQVPPQVIQLKASATPVSVRQYPLSKEAQE
GIRPHVQRLIQQGILVPVQSPWNTPLLPVRKPGTNDYRPVQDLREVNKRVQDIHPTVPNPYNLLCALPPQR
SWYTVLDLKDAFFCLRLHPTSQPLFAFEWRDPGTGRTGQLTWTRLPQGFKNSPTIFDEALHRDLANFRIQH
PQVTLLQYVDDLLLAGATKQDCLEGTKALLLELSDLGYRASAKKAQICRREVTYLGYSLRDGQRWLTEARK
KTVVQIPAPTTAKQVREFLGTAGFCRLWIPGFATLAAPLYPLTKEKGEFSWAPEHQKAFDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLLTERVTFAPPAALNPATLLPEETDEPVTHDCHQL
LIEETGVRKDLTDIPL
SEQ ID NO: 7 AC-RT-M3Δ
TLQLDDEYRLYSPLVKPDQNIQFWLEQFPQAWAETAGMGLAKQVPPQVIQLKASATPVSVRQYPLSKEAQE
GIRPHVQRLIQQGILVPVQSPWNTPLLPVRKPGTNDYRPVQDLREVNKRVQDIHPTVPNPYNLLCALPPQR
SWYTVLDLKDAFFCLRLHPTSQPLFAFEWRDPGTGRTGQLTWTRLPQGFKNSPTIFNEALHRDLANFRIQH
PQVTLLQYVDDLLLAGATKQDCLEGTKALLLELSDLGYRASAKKAQICRREVTYLGYSLRDGQRWLTEARK
KTVVQIPAPTTAKQVREFLGKAGFCRLFIPGFATLAAPLYPLTKEKGEFSWAPEHQKAFDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLLTERVTFAPPAALNPATLLPEETDEPVTHDCHQL
LIEETGVRKDLTDIPL
SEQ ID NO: 8 AC-RT-M4Δ
TLQLDDEYRLYSPLVKPDQNIQFWLEQFPQAWAETAGMGLAKQVPPQVIQLKASATPVSVRQYPLSKEAQE
GIRPHVQRLIQQGILVPVQSPWNTPLLPVRKPGTNDYRPVQDLREVNKRVQDIHPTVPNPYNLLCALPPQR
SWYTVLDLKDAFFCLRLHPTSQPLFAFEWRDPGTGRTGQLTWTRLPQGFKNSPTIFNEALHRDLANFRIQH
PQVTLLQYVDDLLLAGATKQDCLEGTKALLLELSDLGYRASAKKAQICRREVTYLGYSLRDGQRWLTEARK
KTVVQIPAPTTAKQVREFLGKAGFCRLFIPGFATLAAPLYPLTKPGTLESWAPEHQKAFDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLLTERVTFAPPAALNPATLLPEETDEPVTHDCHQL
LIEETGVRKDLTDIPL
SEQ ID NO: 9 B-RT-M0Δ
TLQLDDEYRLYSPQVKPDQDIQSWLEQFPQAWAETAGMGLAKQVPPQVIQLKASATPVSVRQYPLSREARE
GIWPHVQRLIQQGILVPVQSPWNTPLLPVRKPGTNDYRPVQDLREVNKRVQDIHPTVPNPYNLLSALPPER
NWYTVLDLKDAFFCLRLHPTSQPLFAFEWRDPGTGRTGQLTWTRLPQGEKNSPTIFDEALHRDLANFRIQH
PQVTLLQYVDDLLLAGATKQDCLEGTKALLLELSDLGYRASAKKAQICRREVTYLGYSLRGGQRWLTEARK
KTVVQIPAPTTAKQVREFLGTAGFCRLWIPGFATLAAPLYPLTKEKGEFSWAPEHQKTEDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLLTERVTFAPPAALNPATLLPEETDEPVTHDCHQL
LIEETGVRKDLTDIPL
SEQ ID NO: 10 B-RT-M3Δ
TLQLDDEYRLYSPQVKPDQDIQSWLEQFPQAWAETAGMGLAKQVPPQVIQLKASATPVSVRQYPLSREARE
GIWPHVQRLIQQGILVPVQSPWNTPLLPVRKPGTNDYRPVQDLREVNKRVQDIHPTVPNPYNLLSALPPER
NWYTVLDLKDAFFCLRLHPTSQPLFAFEWRDPGTGRTGQLTWTRLPQGFKNSPTIFNEALHRDLANFRIQH
PQVTLLQYVDDLLLAGATKQDCLEGTKALLLELSDLGYRASAKKAQICRREVTYLGYSLRGGQRWLTEARK
KTVVQIPAPTTAKQVREFLGKAGFCRLFIPGFATLAAPLYPLTKEKGEFSWAPEHQKTFDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLLTERVTFAPPAALNPATLLPEETDEPVTHDCHQL
LIEETGVRKDLTDIPL
SEQ ID NO: 11 B-RT-M4Δ*
TLQLDDEYRLYSPQVKPDQDIQSWLEQFPQAWAETAGMGLAKQVPPQVIQLKASATPVSVRQYPLSREARE
GIWPHVQRLIQQGILVPVQSPWNTPLLPVRKPGTNDYRPVQDLREVNKRVQDIHPTVPNPYNLLSALPPER
NWYTVLDLKDAFFCLRLHPTSQPLFAFEWRDPGTGRTGQLTWTRLPQGFKNSPTIFNEALHRDLANFRIQH
PQVTLLQYVDDLLLAGATKQDCLEGTKALLLELSDLGYRASAKKAQICRREVTYLGYSLRGGQRWLTEARK
KTVVQIPAPTTAKQVREFLGKAGFCRLFIPGFATLAAPLYPLTKPGTLESWAPEHQKTEDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLLTERVTFAPPAALNPATLLPEETDEPVTHDCHQL
LIEETGVRKDLTDIPL
SEQ ID NO: 12 bmx-A-RT-M4
TLQLDDEYRLYSPLVKPDQNIQFWLEQFPQAWAETAGMGLAKQVPPQVIQLKASATPVSVRQYPLSKEARE
GIRSHVQRLIQQGILVPVQSPWNTPLLPVRKPGTNDYRPVQDLREVNKRVQDIHPTVPNPYNLLCALPPQR
SWYTVLDLKDAFFCLRLHPTSQPLFAFEWRDPGAGRTGQLTWTRLPQGFKNSPTIFNEALHRDLANFRIQH
PQVTLLQYVDDLLLAGATKQDCLEGTKALLLELSDLGYRASAKKAQICRREVTYLGYSLRGGQRWLMEARK
KTVVQIPAPTTAKQVREFLGKAGFCRLFIPGFATLAAPLYPLTKEKGEFSWAPEHQKAFDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLLTERVTFAPPAALNPATLLPEEADEPVTHDCHQL
LIEETGVRKDLTDIPLTGEVLTWFTDRSSYVVEGKRMAGAAVVDGTHTIWASSLPEGTSAQKAELMALTQA
LRLAEGKSINIYTDSRYAFATAHVHRAIYKQRGWLTSAGREIKNKEEILSLLEALHLPKRLAIIHCPGHQK
AKDPISRGNQMADRVTKQAAQGVNLLPIIETPKAPEP
Note: underlined is the RNase H domain
SEQ ID NO: 13 bmx-B-RT-M4
TLQLDDEYRLYSPQVKPDQDIQSWLEQFPQAWAETAGMGLAKQVPPQVIQLKASATPVSVRQYPLSREARE
GIWPHVQRLIQQGILVPVQSPWNTPLLPVRKPGTNDYRPVQDLREVNKRVQDIHPTVPNPYNLLSALPPEQ
NWYTVLDLKDAFFCLRLHPTSQPLFAFKWRDPGTGRTGQLTWTRLPQGFKNSPTIFNEALHRDLANFRIQH
PQVTLLQYVDDLLLAGATKQDCLEGTKALLLELSDLGYRASAKKAQICRREVTYLGYSLRGGQRWLTEARK
KTVVQIPAPTTAKQVREFLGKAGFCRLFIPGFATLAAPLYLLTKEKGEFSWAPEHQKAFDAIKKSLLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNACMTHYQSLLLTERVTFAPPAALNPATLLPEETDEPVTHDCHQL
LIEETGVRKDLTDIPLTGEVLTWFTDGSSYVVEGKRMAGAAVVDGTRTIWASSLPEGTSAQKAELMALTQA
LRLAEGKSINIYTDSRYAFATAHVHGAIYKQRGWLTSAGREIKNKEEILSLLEALHLPKRLAIIHCPGHQK
AKDLISRGNQMADRVAKQAAQAVNLLPIIETPKAPEP
Note: underlined is the RNase H domain
SEQ ID NO: 14 bmx-C-RT-M4
TLQLDDEYRLYSPLVKPDQNIQFWLEQFPQAWAETAGVGLAKQVPPQVIQLKASATPVSVRQYPLSKEARE
GIRPHVQRLIQQGILVPVQSPWNTPLLPVRKPGTNDYRPVQDLREVNKRVQDIHPTVPNPYNLLCALPPQR
SWYTVLDLKDAFFCLRLHPTSQPLFAFEWRDPGAGRTGQLTWTRLPQGFKNSPTIFNEALHRDLANFRIQH
PQVTLLQYVDDLLLAGATKQDCLEGTKALLLELSDLGYRASAKKAQICRREVTYLGYSLRGGQRWLTEARK
RTVVQIPAPTTAKQVREFLGKAGFCRLFIPGFATLAAPLYPLTKEKGEFSWAPEHQKAFDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLLTERVTFAPPAALNPATLLPEETDEPVTHDCHQL
LIEETGVRKDLTDIPLTGEVLTWFTDGSSYVVEGKRMAGAAVVDGTRTIWASSLPEGTSAQKAELMALTQA
LRLAEGKSINIYTDSRYAFATAHVHGAIYKQRGWLTSAGREIKNKEEILSLLEALHLPKRLAIIHCPGHQK
AKDPISRGNQMADRVAKQAAQGVNLLPIIETPKAPEP
Note: underlined is the RNase H domain
SEQ ID NO: 15 bmx-A-RT-M3Δ
TLQLDDEYRLYSPLVKPDQNIQFWLEQFPQAWAETAGMGLAKQVPPQVIQLKASATPVSVRQYPLSKEARE
GIRSHVQRLIQQGILVPVQSPWNTPLLPVRKPGTNDYRPVQDLREVNKRVQDIHPTVPNPYNLLCALPPQR
SWYTVLDLKDAFFCLRLHPTSQPLFAFEWRDPGAGRTGQLTWTRLPQGFKNSPTIFNEALHRDLANFRIQH
PQVTLLQYVDDLLLAGATKQDCLEGTKALLLELSDLGYRASAKKAQICRREVTYLGYSLRGGQRWLMEARK
KTVVQIPAPTTAKQVREFLGKAGFCRLFIPGFATLAAPLYPLTKEKGEFSWAPEHQKAFDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLLTERVTFAPPAALNPATLLPEEADEPVTHDCHQL
LIEETGVRKDLTDIPL
SEQ ID NO: 16 bmx-B-RT-M3Δ
TLQLDDEYRLYSPQVKPDQDIQSWLEQFPQAWAETAGMGLAKQVPPQVIQLKASATPVSVRQYPLSREARE
GIWPHVQRLIQQGILVPVQSPWNTPLLPVRKPGTNDYRPVQDLREVNKRVQDIHPTVPNPYNLLSALPPEQ
NWYTVLDLKDAFFCLRLHPTSQPLFAFKWRDPGTGRTGQLTWTRLPQGFKNSPTIFNEALHRDLANFRIQH
PQVTLLQYVDDLLLAGATKQDCLEGTKALLLELSDLGYRASAKKAQICRREVTYLGYSLRGGQRWLTEARK
KTVVQIPAPTTAKQVREFLGKAGFCRLFIPGFATLAAPLYLLTKEKGEFSWAPEHQKAFDAIKKSLLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNACMTHYQSLLLTERVTFAPPAALNPATLLPEETDEPVTHDCHQL
LIEETGVRKDLTDIPL
SEQ ID NO: 17 bmx-C-RT-M3Δ
TLQLDDEYRLYSPLVKPDQNIQFWLEQFPQAWAETAGVGLAKQVPPQVIQLKASATPVSVRQYPLSKEARE
GIRPHVQRLIQQGILVPVQSPWNTPLLPVRKPGTNDYRPVQDLREVNKRVQDIHPTVPNPYNLLCALPPQR
SWYTVLDLKDAFFCLRLHPTSQPLFAFEWRDPGAGRTGQLTWTRLPQGFKNSPTIFNEALHRDLANFRIQH
PQVTLLQYVDDLLLAGATKQDCLEGTKALLLELSDLGYRASAKKAQICRREVTYLGYSLRGGQRWLTEARK
RTVVQIPAPTTAKQVREFLGKAGFCRLFIPGFATLAAPLYPLTKEKGEFSWAPEHQKAFDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLLTERVTFAPPAALNPATLLPEETDEPVTHDCHQL
LIEETGVRKDLTDIPL
SEQ ID NO: 18 SpCas9 (H840A)
MKRTADGSEFESPKKKRKVDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFDLAEDAKLQL
SKDTYDDDLDNLLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKIIKDKDFLDNEENEDILEDIVL
TLTLFEDREMIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVSDFRKDFQFYKVR
EINNYHHAHDAYLNAVVGTALIKKYPKLESEFVYGDYKVYDVRKMIAKSEQEIGKATAKYFFYSNIMNFFK
TEITLANGEIRKRPLIETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAAFKYFDTTI
DRKRYTSTKEVLDATLIHQSITGLYETRIDLSQLGGD
SEQ ID NO: 19 PERV-AC-NC
DFRKIRSGPRQSGNLGNRTPLDKDQCAYCKEKGHWARNCPKKGNKGLKVLALEEDK
SEQ ID NO: 20 XTEN
SGGSSGGSSGSETPGTSESATPESSGGSSGGSS
SEQ ID NO: 21 gRNA scaffold
GTTTTAGAGCTAGAAATAGCAAGTTAAAATAAGGCTAGTCCGTTATCAACTTGAAAAAGTGGCACCGAGTC
GGTGC
SEQ ID NO: 22 epPE-AC-M3Δ
MKRTADGSEFESPKKKRKVDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESELVEEDKKHERHPIFGNIVD
EVAYHEKYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNEKSNFDLAEDAKLQL
SKDTYDDDLDNLLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKIIKDKDFLDNEENEDILEDIVL
TLTLFEDREMIEERLKTYAHLFDDKVMKQLKRRRYTGWGRLSRKLINGIRDKQSGKTILDELKSDGFANRN
FMQLIHDDSLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVSDFRKDFQFYKVR
EINNYHHAHDAYLNAVVGTALIKKYPKLESEFVYGDYKVYDVRKMIAKSEQEIGKATAKYFFYSNIMNFFK
TEITLANGEIRKRPLIETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAAFKYEDTTI
DRKRYTSTKEVLDATLIHQSITGLYETRIDLSQLGGDSGGSSGGSSGSETPGTSESATPESSGGSSGGSSD
FRKIRSGPRQSGNLGNRTPLDKDQCAYCKEKGHWARNCPKKGNKGLKVLALEEDKSGSETPGTSESATPES
TLQLDDEYRLYSPLVKPDQNIQFWLEQFPQAWAETAGMGLAKQVPPQVIQLKASATPVSVRQYPLSKEAQE
GIRPHVQRLIQQGILVPVQSPWNTPLLPVRKPGTNDYRPVQDLREVNKRVQDIHPTVPNPYNLLCALPPQR
SWYTVLDLKDAFFCLRLHPTSQPLFAFEWRDPGTGRTGQLTWTRLPQGFKNSPTIFNEALHRDLANFRIQH
PQVTLLQYVDDLLLAGATKQDCLEGTKALLLELSDLGYRASAKKAQICRREVTYLGYSLRDGQRWLTEARK
KTVVQIPAPTTAKQVREFLGKAGFCRLFIPGFATLAAPLYPLTKEKGEFSWAPEHQKAFDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLLTERVTFAPPAALNPATLLPEETDEPVTHDCHQL
LIEETGVRKDLTDIPLSGGSKRTADGSEFEPKKKRKV
SEQ ID NO:23 epPE-AC-M4Δ*
MKRTADGSEFESPKKKRKVDKKYSIGLDIGTNSVGWAVITDEYKVPSKKFKVLGNTDRHSIKKNLIGALLE
DSGETAEATRLKRTARRRYTRRKNRICYLQEIFSNEMAKVDDSFFHRLEESELVEEDKKHERHPIFGNIVD
EVAYHEKYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNEKSNFDLAEDAKLQL
SKDTYDDDLDNLLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKIIKDKDFLDNEENEDILEDIVL
TLTLFEDREMIEERLKTYAHLFDDKVMKQLKRRRYTGWGRLSRKLINGIRDKQSGKTILDELKSDGFANRN
FMQLIHDDSLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVSDFRKDFQFYKVR
EINNYHHAHDAYLNAVVGTALIKKYPKLESEFVYGDYKVYDVRKMIAKSEQEIGKATAKYFFYSNIMNFFK
TEITLANGEIRKRPLIETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEKNPIDELEAKGYKE
VKKDLIIKLPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAAFKYEDTTI
DRKRYTSTKEVLDATLIHQSITGLYETRIDLSQLGGDSGGSSGGSSGSETPGTSESATPESSGGSSGGSSD
FRKIRSGPRQSGNLGNRTPLDKDQCAYCKEKGHWARNCPKKGNKGLKVLALEEDKSGSETPGTSESATPES
TLQLDDEYRLYSPLVKPDQNIQFWLEQFPQAWAETAGMGLAKQVPPQVIQLKASATPVSVRQYPLSKEAQE
GIRPHVQRLIQQGILVPVQSPWNTPLLPVRKPGTNDYRPVQDLREVNKRVQDIHPTVPNPYNLLCALPPQR
SWYTVLDLKDAFFCLRLHPTSQPLFAFEWRDPGTGRTGQLTWTRLPQGFKNSPTIFNEALHRDLANFRIQH
PQVTLLQYVDDLLLAGATKQDCLEGTKALLLELSDLGYRASAKKAQICRREVTYLGYSLRDGQRWLTEARK
KTVVQIPAPTTAKQVREFLGKAGFCRLFIPGFATLAAPLYPLTKPGTLESWAPEHQKAFDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLLTERVTFAPPAALNPATLLPEETDEPVTHDCHQL
LIEETGVRKDLTDIPLSGGSKRTADGSEFEPKKKRKV
SEQ ID NO: 24 epPE-B-M3Δ
MKRTADGSEFESPKKKRKVDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESELVEEDKKHERHPIFGNIVD
EVAYHEKYPTIYHLRKKLVDSTDKADLRLIYLALAHMIKERGHFLIEGDLNPDNSDVDKLFIQLVQTYNQL
FEENPINASGVDAKAILSARLSKSRRLENLIAQLPGEKKNGLFGNLIALSLGLTPNEKSNFDLAEDAKLQL
SKDTYDDDLDNLLAQIGDQYADLELAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRENASLGTYHDLLKIIKDKDFLDNEENEDILEDIVL
TLTLFEDREMIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQLLNAKLITQRKEDNLTKAER
GGLSELDKAGFIKRQLVETRQITKHVAQILDSRMNTKYDENDKLIREVKVITLKSKLVSDERKDFQFYKVR
EINNYHHAHDAYLNAVVGTALIKKYPKLESEFVYGDYKVYDVRKMIAKSEQEIGKATAKYFFYSNIMNFFK
TEITLANGEIRKRPLIETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAAFKYEDTTI
DRKRYTSTKEVLDATLIHQSITGLYETRIDLSQLGGDSGGSSGGSSGSETPGTSESATPESSGGSSGGSSD
FRKIRSGPRQSGNLGNRTPLDKDQCAYCKEKGHWARNCPKKGNKGPKVLALEEDKSGSETPGTSESATPES
TLQLDDEYRLYSPQVKPDQDIQSWLEQFPQAWAETAGMGLAKQVPPQVIQLKASATPVSVRQYPLSREARE
GIWPHVQRLIQQGILVPVQSPWNTPLLPVRKPGTNDYRPVQDLREVNKRVQDIHPTVPNPYNLLSALPPER
NWYTVLDLKDAFFCLRLHPTSQPLFAFEWRDPGTGRTGQLTWTRLPQGFKNSPTIFNEALHRDLANFRIQH
PQVTLLQYVDDLLLAGATKQDCLEGTKALLLELSDLGYRASAKKAQICRREVTYLGYSLRGGQRWLTEARK
KTVVQIPAPTTAKQVREFLGKAGFCRLFIPGFATLAAPLYPLTKEKGEFSWAPEHQKTEDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLLTERVTFAPPAALNPATLLPEETDEPVTHDCHQL
LIEETGVRKDLTDIPLSGGSKRTADGSEFEPKKKRKV
SEQ ID NO: 25 epPE-B-M4Δ*
MKRTADGSEFESPKKKRKVDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESELVEEDKKHERHPIFGNIVD
EVAYHEKYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNEKSNFDLAEDAKLQL
SKDTYDDDLDNLLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLLRKQRTFDNG
SIPHQIHLGELHAILRRQEDFYPELKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRENASLGTYHDLLKIIKDKDFLDNEENEDILEDIVL
TLTLFEDREMIEERLKTYAHLEDDKVMKQLKRRRYTGWGRLSRKLINGIRDKQSGKTILDELKSDGFANRN
FMQLIHDDSLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVSDFRKDFQFYKVR
EINNYHHAHDAYLNAVVGTALIKKYPKLESEFVYGDYKVYDVRKMIAKSEQEIGKATAKYFFYSNIMNFFK
TEITLANGEIRKRPLIETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAAFKYEDTTI
DRKRYTSTKEVLDATLIHQSITGLYETRIDLSQLGGDSGGSSGGSSGSETPGTSESATPESSGGSSGGSSD
FRKIRSGPRQSGNLGNRTPLDKDQCAYCKEKGHWARNCPKKGNKGPKVLALEEDKSGSETPGTSESATPES
TLQLDDEYRLYSPQVKPDQDIQSWLEQFPQAWAETAGMGLAKQVPPQVIQLKASATPVSVRQYPLSREARE
GIWPHVQRLIQQGILVPVQSPWNTPLLPVRKPGTNDYRPVQDLREVNKRVQDIHPTVPNPYNLLSALPPER
NWYTVLDLKDAFFCLRLHPTSQPLFAFEWRDPGTGRTGQLTWTRLPQGFKNSPTIFNEALHRDLANFRIQH
PQVTLLQYVDDLLLAGATKQDCLEGTKALLLELSDLGYRASAKKAQICRREVTYLGYSLRGGQRWLTEARK
KTVVQIPAPTTAKQVREFLGKAGFCRLFIPGFATLAAPLYPLTKPGTLESWAPEHQKTEDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLLTERVTFAPPAALNPATLLPEETDEPVTHDCHQL
LIEETGVRKDLTDIPLSGGSKRTADGSEFEPKKKRKV
SEQ ID NO: 26 bmx-pPE-A-M0
MKRTADGSEFESPKKKRKVDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKERGHFLIEGDLNPDNSDVDKLFIQLVQTYNQL
FEENPINASGVDAKAILSARLSKSRRLENLIAQLPGEKKNGLFGNLIALSLGLTPNFKSNFDLAEDAKLQL
SKDTYDDDLDNLLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKIIKDKDELDNEENEDILEDIVL
TLTLFEDREMIEERLKTYAHLFDDKVMKQLKRRRYTGWGRLSRKLINGIRDKQSGKTILDELKSDGFANRN
FMQLIHDDSLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVSDERKDFQFYKVR
EINNYHHAHDAYLNAVVGTALIKKYPKLESEFVYGDYKVYDVRKMIAKSEQEIGKATAKYFFYSNIMNFFK
TEITLANGEIRKRPLIETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAAFKYFDTTI
DRKRYTSTKEVLDATLIHQSITGLYETRIDLSQLGGDSGGSSGGSSGSETPGTSESATPESSGGSSGGSST
LQLDDEYRLYSPLVKPDQNIQFWLEQFPQAWAETAGMGLAKQVPPQVIQLKASATPVSVRQYPLSKEAREG
IRSHVQRLIQQGILVPVQSPWNTPLLPVRKPGTNDYRPVQDLREVNKRVQDIHPTVPNPYNLLCALPPQRS
WYTVLDLKDAFFCLRLHPTSQPLFAFEWRDPGAGRTGQLTWTRLPQGFKNSPTIFDEALHRDLANFRIQHP
QVTLLQYVDDLLLAGATKQDCLEGTKALLLELSDLGYRASAKKAQICRREVTYLGYSLRGGQRWLMEARKK
TVVQIPAPTTAKQVREFLGTAGFCRLWIPGFATLAAPLYPLTKEKGEFSWAPEHQKAFDAIKKALLSAPAL
ALPDVTKPFTLYVDERKGVARGVLTQTLGPWRRPVAYLSKKLDPVASGWPVCLKAIAAVAILVKDADKLTL
GQNITVIAPHALENIVRQPPDRWMTNARMTHYQSLLLTERVTFAPPAALNPATLLPEEADEPVTHDCHQLL
IEETGVRKDLTDIPLTGEVLTWFTDRSSYVVEGKRMAGAAVVDGTHTIWASSLPEGTSAQKAELMALTQAL
RLAEGKSINIYTDSRYAFATAHVHRAIYKQRGLLTSAGREIKNKEEILSLLEALHLPKRLAIIHCPGHQKA
KDPISRGNQMADRVTKQAAQGVNLLPIIETPKAPEPSGGSKRTADGSEFEPKKKRKV
SEQ ID NO: 27 bmx-pPE-B-M0
MKRTADGSEFESPKKKRKVDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFDLAEDAKLQL
SKDTYDDDLDNLLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRENASLGTYHDLLKIIKDKDELDNEENEDILEDIVL
TLTLFEDREMIEERLKTYAHLEDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVSDFRKDFQFYKVR
EINNYHHAHDAYLNAVVGTALIKKYPKLESEFVYGDYKVYDVRKMIAKSEQEIGKATAKYFFYSNIMNFFK
TEITLANGEIRKRPLIETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAAFKYEDTTI
DRKRYTSTKEVLDATLIHQSITGLYETRIDLSQLGGDSGGSSGGSSGSETPGTSESATPESSGGSSGGSST
LQLDDEYRLYSPQVKPDQDIQSWLEQFPQAWAETAGMGLAKQVPPQVIQLKASATPVSVRQYPLSREAREG
IWPHVQRLIQQGILVPVQSPWNTPLLPVRKPGTNDYRPVQDLREVNKRVQDIHPTVPNPYNLLSALPPEQN
WYTVLDLKDAFFCLRLHPTSQPLFAFKWRDPGTGRTGQLTWTRLPQGFKNSPTIFDEALHRDLANFRIQHP
QVTLLQYVDDLLLAGATKQDCLEGTKALLLELSDLGYRASAKKAQICRREVTYLGYSLRGGQRWLTEARKK
TVVQIPAPTTAKQVREFLGTAGFCRLWIPGFATLAAPLYLLTKEKGEFSWAPEHQKAFDAIKKSLLSAPAL
ALPDVTKPFTLYVDERKGVARGVLTQTLGPWRRPVAYLSKKLDPVASGWPVCLKAIAAVAILVKDADKLTL
GQNITVIAPHALENIVRQPPDRWMTNACMTHYQSLLLTERVTFAPPAALNPATLLPEETDEPVTHDCHQLL
IEETGVRKDLTDIPLTGEVLTWFTDGSSYVVEGKRMAGAAVVDGTRTIWASSLPEGTSAQKAELMALTQAL
RLAEGKSINIYTDSRYAFATAHVHGAIYKQRGLLTSAGREIKNKEEILSLLEALHLPKRLAIIHCPGHQKA
KDLISRGNQMADRVAKQAAQAVNLLPIIETPKAPEPSGGSKRTADGSEFEPKKKRKV
SEQ ID NO: 28 bmx-pPE-C-M0
MKRTADGSEFESPKKKRKVDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNEKSNFDLAEDAKLQL
SKDTYDDDLDNLLAQIGDQYADLELAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRENASLGTYHDLLKIIKDKDELDNEENEDILEDIVL
TLTLFEDREMIEERLKTYAHLFDDKVMKQLKRRRYTGWGRLSRKLINGIRDKQSGKTILDELKSDGFANRN
FMQLIHDDSLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVSDFRKDFQFYKVR
EINNYHHAHDAYLNAVVGTALIKKYPKLESEFVYGDYKVYDVRKMIAKSEQEIGKATAKYFFYSNIMNFFK
TEITLANGEIRKRPLIETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAAFKYFDTTI
DRKRYTSTKEVLDATLIHQSITGLYETRIDLSQLGGDSGGSSGGSSGSETPGTSESATPESSGGSSGGSST
LQLDDEYRLYSPLVKPDQNIQFWLEQFPQAWAETAGVGLAKQVPPQVIQLKASATPVSVRQYPLSKEAREG
IRPHVQRLIQQGILVPVQSPWNTPLLPVRKPGTNDYRPVQDLREVNKRVQDIHPTVPNPYNLLCALPPQRS
WYTVLDLKDAFFCLRLHPTSQPLFAFEWRDPGAGRTGQLTWTRLPQGFKNSPTIFDEALHRDLANFRIQHP
QVTLLQYVDDLLLAGATKQDCLEGTKALLLELSDLGYRASAKKAQICRREVTYLGYSLRGGQRWLTEARKR
TVVQIPAPTTAKQVREFLGTAGFCRLWIPGFATLAAPLYPLTKEKGEFSWAPEHQKAFDAIKKALLSAPAL
ALPDVTKPFTLYVDERKGVARGVLTQTLGPWRRPVAYLSKKLDPVASGWPVCLKAIAAVAILVKDADKLTL
GQNITVIAPHALENIVRQPPDRWMTNARMTHYQSLLLTERVTFAPPAALNPATLLPEETDEPVTHDCHQLL
IEETGVRKDLTDIPLTGEVLTWFTDGSSYVVEGKRMAGAAVVDGTRTIWASSLPEGTSAQKAELMALTQAL
RLAEGKSINIYTDSRYAFATAHVHGAIYKQRGLLTSAGREIKNKEEILSLLEALHLPKRLAIIHCPGHQKA
KDPISRGNQMADRVAKQAAQGVNLLPIIETPKAPEPSGGSKRTADGSEFEPKKKRKV
SEQ ID NO: 29 bmx-pPE-A-M4
MKRTADGSEFESPKKKRKVDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFDLAEDAKLQL
SKDTYDDDLDNLLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRENASLGTYHDLLKIIKDKDELDNEENEDILEDIVL
TLTLFEDREMIEERLKTYAHLFDDKVMKQLKRRRYTGWGRLSRKLINGIRDKQSGKTILDELKSDGFANRN
FMQLIHDDSLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVSDERKDFQFYKVR
EINNYHHAHDAYLNAVVGTALIKKYPKLESEFVYGDYKVYDVRKMIAKSEQEIGKATAKYFFYSNIMNFFK
TEITLANGEIRKRPLIETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAAFKYFDTTI
DRKRYTSTKEVLDATLIHQSITGLYETRIDLSQLGGDSGGSSGGSSGSETPGTSESATPESSGGSSGGSST
LQLDDEYRLYSPLVKPDQNIQFWLEQFPQAWAETAGMGLAKQVPPQVIQLKASATPVSVRQYPLSKEAREG
IRSHVQRLIQQGILVPVQSPWNTPLLPVRKPGTNDYRPVQDLREVNKRVQDIHPTVPNPYNLLCALPPQRS
WYTVLDLKDAFFCLRLHPTSQPLFAFEWRDPGAGRTGQLTWTRLPQGFKNSPTIFNEALHRDLANFRIQHP
QVTLLQYVDDLLLAGATKQDCLEGTKALLLELSDLGYRASAKKAQICRREVTYLGYSLRGGQRWLMEARKK
TVVQIPAPTTAKQVREFLGKAGFCRLFIPGFATLAAPLYPLTKEKGEFSWAPEHQKAFDAIKKALLSAPAL
ALPDVTKPFTLYVDERKGVARGVLTQTLGPWRRPVAYLSKKLDPVASGWPVCLKAIAAVAILVKDADKLTL
GQNITVIAPHALENIVRQPPDRWMTNARMTHYQSLLLTERVTFAPPAALNPATLLPEEADEPVTHDCHQLL
IEETGVRKDLTDIPLTGEVLTWFTDRSSYVVEGKRMAGAAVVDGTHTIWASSLPEGTSAQKAELMALTQAL
RLAEGKSINIYTDSRYAFATAHVHRAIYKQRGWLTSAGREIKNKEEILSLLEALHLPKRLAIIHCPGHQKA
KDPISRGNQMADRVTKQAAQGVNLLPIIETPKAPEPSGGSKRTADGSEFEPKKKRKV
SEQ ID NO: 30 bmx-pPE-B-M4
MKRTADGSEFESPKKKRKVDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESELVEEDKKHERHPIFGNIVD
EVAYHEKYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNEKSNFDLAEDAKLQL
SKDTYDDDLDNLLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKIIKDKDELDNEENEDILEDIVL
TLTLFEDREMIEERLKTYAHLEDDKVMKQLKRRRYTGWGRLSRKLINGIRDKQSGKTILDELKSDGFANRN
FMQLIHDDSLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVSDERKDFQFYKVR
EINNYHHAHDAYLNAVVGTALIKKYPKLESEFVYGDYKVYDVRKMIAKSEQEIGKATAKYFFYSNIMNFFK
TEITLANGEIRKRPLIETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAAFKYFDTTI
DRKRYTSTKEVLDATLIHQSITGLYETRIDLSQLGGDSGGSSGGSSGSETPGTSESATPESSGGSSGGSST
LQLDDEYRLYSPQVKPDQDIQSWLEQFPQAWAETAGMGLAKQVPPQVIQLKASATPVSVRQYPLSREAREG
IWPHVQRLIQQGILVPVQSPWNTPLLPVRKPGTNDYRPVQDLREVNKRVQDIHPTVPNPYNLLSALPPEQN
WYTVLDLKDAFFCLRLHPTSQPLFAFKWRDPGTGRTGQLTWTRLPQGFKNSPTIFNEALHRDLANFRIQHP
QVTLLQYVDDLLLAGATKQDCLEGTKALLLELSDLGYRASAKKAQICRREVTYLGYSLRGGQRWLTEARKK
TVVQIPAPTTAKQVREFLGKAGFCRLFIPGFATLAAPLYLLTKEKGEFSWAPEHQKAFDAIKKSLLSAPAL
ALPDVTKPFTLYVDERKGVARGVLTQTLGPWRRPVAYLSKKLDPVASGWPVCLKAIAAVAILVKDADKLTL
GQNITVIAPHALENIVRQPPDRWMTNACMTHYQSLLLTERVTFAPPAALNPATLLPEETDEPVTHDCHQLL
IEETGVRKDLTDIPLTGEVLTWFTDGSSYVVEGKRMAGAAVVDGTRTIWASSLPEGTSAQKAELMALTQAL
RLAEGKSINIYTDSRYAFATAHVHGAIYKQRGWLTSAGREIKNKEEILSLLEALHLPKRLAIIHCPGHQKA
KDLISRGNQMADRVAKQAAQAVNLLPIIETPKAPEPSGGSKRTADGSEFEPKKKRKV
SEQ ID NO: 31 bmx-pPE-C-M4
MKRTADGSEFESPKKKRKVDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESELVEEDKKHERHPIFGNIVD
EVAYHEKYPTIYHLRKKLVDSTDKADLRLIYLALAHMIKERGHFLIEGDLNPDNSDVDKLFIQLVQTYNQL
FEENPINASGVDAKAILSARLSKSRRLENLIAQLPGEKKNGLFGNLIALSLGLTPNFKSNFDLAEDAKLQL
SKDTYDDDLDNLLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRENASLGTYHDLLKIIKDKDELDNEENEDILEDIVL
TLTLFEDREMIEERLKTYAHLFDDKVMKQLKRRRYTGWGRLSRKLINGIRDKQSGKTILDELKSDGFANRN
FMQLIHDDSLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVSDFRKDFQFYKVR
EINNYHHAHDAYLNAVVGTALIKKYPKLESEFVYGDYKVYDVRKMIAKSEQEIGKATAKYFFYSNIMNFFK
TEITLANGEIRKRPLIETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAAFKYFDTTI
DRKRYTSTKEVLDATLIHQSITGLYETRIDLSQLGGDSGGSSGGSSGSETPGTSESATPESSGGSSGGSST
LQLDDEYRLYSPLVKPDQNIQFWLEQFPQAWAETAGVGLAKQVPPQVIQLKASATPVSVRQYPLSKEAREG
IRPHVQRLIQQGILVPVQSPWNTPLLPVRKPGTNDYRPVQDLREVNKRVQDIHPTVPNPYNLLCALPPQRS
WYTVLDLKDAFFCLRLHPTSQPLFAFEWRDPGAGRTGQLTWTRLPQGEKNSPTIFNEALHRDLANFRIQHP
QVTLLQYVDDLLLAGATKQDCLEGTKALLLELSDLGYRASAKKAQICRREVTYLGYSLRGGQRWLTEARKR
TVVQIPAPTTAKQVREFLGKAGFCRLFIPGFATLAAPLYPLTKEKGEFSWAPEHQKAFDAIKKALLSAPAL
ALPDVTKPFTLYVDERKGVARGVLTQTLGPWRRPVAYLSKKLDPVASGWPVCLKAIAAVAILVKDADKLTL
GQNITVIAPHALENIVRQPPDRWMTNARMTHYQSLLLTERVTFAPPAALNPATLLPEETDEPVTHDCHQLL
IEETGVRKDLTDIPLTGEVLTWFTDGSSYVVEGKRMAGAAVVDGTRTIWASSLPEGTSAQKAELMALTQAL
RLAEGKSINIYTDSRYAFATAHVHGAIYKQRGWLTSAGREIKNKEEILSLLEALHLPKRLAIIHCPGHQKA
KDPISRGNQMADRVAKQAAQGVNLLPIIETPKAPEPSGGSKRTADGSEFEPKKKRKV
SEQ ID NO: 32 bmx-epPE-A-M3Δ
MKRTADGSEFESPKKKRKVDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNEKSNFDLAEDAKLQL
SKDTYDDDLDNLLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRENASLGTYHDLLKIIKDKDELDNEENEDILEDIVL
TLTLFEDREMIEERLKTYAHLEDDKVMKQLKRRRYTGWGRLSRKLINGIRDKQSGKTILDELKSDGFANRN
FMQLIHDDSLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQLLNAKLITQRKEDNLTKAER
GGLSELDKAGFIKRQLVETRQITKHVAQILDSRMNTKYDENDKLIREVKVITLKSKLVSDERKDFQFYKVR
EINNYHHAHDAYLNAVVGTALIKKYPKLESEFVYGDYKVYDVRKMIAKSEQEIGKATAKYFFYSNIMNFFK
TEITLANGEIRKRPLIETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAAFKYFDTTI
DRKRYTSTKEVLDATLIHQSITGLYETRIDLSQLGGDSGGSSGGSSGSETPGTSESATPESSGGSSGGSSD
FRKIRSGPRQSGNLGNRTPLDKDQCAYCKEKGHWARDCPKKGNKGLKVLALEEDKSGSETPGTSESATPES
TLQLDDEYRLYSPLVKPDQNIQFWLEQFPQAWAETAGMGLAKQVPPQVIQLKASATPVSVRQYPLSKEARE
GIRSHVQRLIQQGILVPVQSPWNTPLLPVRKPGTNDYRPVQDLREVNKRVQDIHPTVPNPYNLLCALPPQR
SWYTVLDLKDAFFCLRLHPTSQPLFAFEWRDPGAGRTGQLTWTRLPQGFKNSPTIFNEALHRDLANFRIQH
PQVTLLQYVDDLLLAGATKQDCLEGTKALLLELSDLGYRASAKKAQICRREVTYLGYSLRGGQRWLMEARK
KTVVQIPAPTTAKQVREFLGKAGFCRLFIPGFATLAAPLYPLTKEKGEFSWAPEHQKAFDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLLTERVTFAPPAALNPATLLPEEADEPVTHDCHQL
LIEETGVRKDLTDIPLTGEVLTWFTDRSSYVVEGKRMAGAAVVDGTHTIWASSLPEGTSAQKAELMALTQA
LRLAEGKSINIYTDSRYAFATAHVHRAIYKQRGWLTSAGREIKNKEEILSLLEALHLPKRLAIIHCPGHQK
AKDPISRGNQMADRVTKQAAQGVNLLPIIETPKAPEPSGGSKRTADGSEFEPKKKRKV
SEQ ID NO: 33 bmx-epPE-B-M3Δ
MKRTADGSEFESPKKKRKVDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESELVEEDKKHERHPIFGNIVD
EVAYHEKYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFDLAEDAKLQL
SKDTYDDDLDNLLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKIIKDKDELDNEENEDILEDIVL
TLTLFEDREMIEERLKTYAHLEDDKVMKQLKRRRYTGWGRLSRKLINGIRDKQSGKTILDELKSDGFANRN
FMQLIHDDSLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQLLNAKLITQRKEDNLTKAER
GGLSELDKAGFIKRQLVETRQITKHVAQILDSRMNTKYDENDKLIREVKVITLKSKLVSDFRKDFQFYKVR
EINNYHHAHDAYLNAVVGTALIKKYPKLESEFVYGDYKVYDVRKMIAKSEQEIGKATAKYFFYSNIMNEFK
TEITLANGEIRKRPLIETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQLEVE
QHKHYLDEIIEQISEFSKRVILADANLDKVLSAYNKHRDKPIREQAENIIHLFTLTNLGAPAAFKYFDTTI
DRKRYTSTKEVLDATLIHQSITGLYETRIDLSQLGGDSGGSSGGSSGSETPGTSESATPESSGGSSGGSSD
FRKIRSGPRQSGNLGNRTPLDKDQCAYCKKKGHWARNCPKKGNKGPKVLALEEDKSGSETPGTSESATPES
TLQLDDEYRLYSPQVKPDQDIQSWLEQFPQAWAETAGMGLAKQVPPQVIQLKASATPVSVRQYPLSREARE
GIWPHVQRLIQQGILVPVQSPWNTPLLPVRKPGTNDYRPVQDLREVNKRVQDIHPTVPNPYNLLSALPPEQ
NWYTVLDLKDAFFCLRLHPTSQPLFAFKWRDPGTGRTGQLTWTRLPQGFKNSPTIFNEALHRDLANFRIQH
PQVTLLQYVDDLLLAGATKQDCLEGTKALLLELSDLGYRASAKKAQICRREVTYLGYSLRGGQRWLTEARK
KTVVQIPAPTTAKQVREFLGKAGFCRLFIPGFATLAAPLYLLTKEKGEFSWAPEHQKAFDAIKKSLLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNACMTHYQSLLLTERVTFAPPAALNPATLLPEETDEPVTHDCHQL
LIEETGVRKDLTDIPLTGEVLTWFTDGSSYVVEGKRMAGAAVVDGTRTIWASSLPEGTSAQKAELMALTQA
LRLAEGKSINIYTDSRYAFATAHVHGAIYKQRGWLTSAGREIKNKEEILSLLEALHLPKRLAIIHCPGHQK
AKDLISRGNQMADRVAKQAAQAVNLLPIIETPKAPEPSGGSKRTADGSEFEPKKKRKV
SEQ ID NO:3 4 bmx-epPE-C-M3Δ
MKRTADGSEFESPKKKRKVDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESELVEEDKKHERHPIFGNIVD
EVAYHEKYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNEKSNFDLAEDAKLQL
SKDTYDDDLDNLLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKIIKDKDELDNEENEDILEDIVL
TLTLFEDREMIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQLLNAKLITQRKEDNLTKAER
GGLSELDKAGFIKRQLVETRQITKHVAQILDSRMNTKYDENDKLIREVKVITLKSKLVSDERKDFQFYKVR
EINNYHHAHDAYLNAVVGTALIKKYPKLESEFVYGDYKVYDVRKMIAKSEQEIGKATAKYFFYSNIMNFFK
TEITLANGEIRKRPLIETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQLEVE
QHKHYLDEIIEQISEFSKRVILADANLDKVLSAYNKHRDKPIREQAENIIHLFTLTNLGAPAAFKYFDTTI
DRKRYTSTKEVLDATLIHQSITGLYETRIDLSQLGGDSGGSSGGSSGSETPGTSESATPESSGGSSGGSSD
FRKIRSGPRQLGNLGNRTPLDKDQCAYCKEKGHWARDCPKKGNKGLKVLALEEDKSGSETPGTSESATPES
TLQLDDEYRLYSPLVKPDQNIQFWLEQFPQAWAETAGVGLAKQVPPQVIQLKASATPVSVRQYPLSKEARE
GIRPHVQRLIQQGILVPVQSPWNTPLLPVRKPGTNDYRPVQDLREVNKRVQDIHPTVPNPYNLLCALPPQR
SWYTVLDLKDAFFCLRLHPTSQPLFAFEWRDPGAGRTGQLTWTRLPQGFKNSPTIFNEALHRDLANFRIQH
PQVTLLQYVDDLLLAGATKQDCLEGTKALLLELSDLGYRASAKKAQICRREVTYLGYSLRGGQRWLTEARK
RTVVQIPAPTTAKQVREFLGKAGFCRLFIPGFATLAAPLYPLTKEKGEFSWAPEHQKAFDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLLTERVTFAPPAALNPATLLPEETDEPVTHDCHQL
LIEETGVRKDLTDIPLTGEVLTWFTDGSSYVVEGKRMAGAAVVDGTRTIWASSLPEGTSAQKAELMALTQA
LRLAEGKSINIYTDSRYAFATAHVHGAIYKQRGWLTSAGREIKNKEEILSLLEALHLPKRLAIIHCPGHQK
AKDPISRGNQMADRVAKQAAQGVNLLPIIETPKAPEPSGGSKRTADGSEFEPKKKRKV
SEQ ID NO: 35 Puro R amino acid sequence
MTEYKPTVRLATRDDVPRAVRTLAAAFADYPATRHTVDPDRHIERVTELQELELTRVGLDIGKVWVADDGA
AVAVWTTPESVEAGAVFAEIGPRMAELSGSRLAAQQQMEGLLAPHRPKEPAWFLATVGVSPDHQGKGLGSA
VVLPGVEAAERAGVPAFLETSAPRNLPFYERLGFTVTADVEVPEGPRTWCMTRKPGA
SEQ ID NO: 36 PERV-B-NC
DERKIRSGPRQSGNLGNRTPLDKDQCAYCKEKGHWARNCPKKGNKGPKVLALEEDK
SEQ ID NO: 37 bmx-PERV-A-NC
DERKIRSGPRQSGNLGNRTPLDKDQCAYCKEKGHWARDCPKKGNKGLKVLALEEDK
SEQ ID NO: 38 bmx-PERV-B-NC
DFRKIRSGPRQSGNLGNRTPLDKDQCAYCKKKGHWARNCPKKGNKGPKVLALEEDK
SEQ ID NO: 39 bmx-PERV-C-NC
DERKIRSGPRQLGNLGNRTPLDKDQCAYCKEKGHWARDCPKKGNKGLKVLALEEDK
SEQ ID NO: 40 bmx-C-RT-M5.1
TLQLDDEYRLYSPLVKPDQNIQFWLEQFPQAWAETAGVGLAKQVPPQVIQLKASATPVSVRQYPLSKEARE
GIRPHVQRLIQQGILVPVQSPWNTPLLPVRKPGTNDYRPVQDLREVNKRVQDIHPTVPNPYNLLCALPPQR
SWYTVLDLKDAFFCLRLHPTSQPLFAFEWRDPGAGRTGQLTWTRLPQGFKNSPTIENEALHRDLANFRIQH
PQVTLLQYADDLLLAGATKQDCLEGTKALLLELSDLGYRASAKKAQICRREVTYLGYSLRGGQRWLTEARK
RTVVQIPAPTTAKQVREFLGKAGFCRLFIPGFATLAAPLYPLTKEKGEFSWAPEHQKAFDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLLTERVTFAPPAALNPATLLPEETDEPVTHDCHQL
LIEETGVRKDLTDIPLTGEVLTWFTDGSSYVVEGKRMAGAAVVDGTRTIWASSLPEGTSAQKAELMALTQA
IRLAEGKSINIYTDSRYAFATAHVAGAIYKQRGWLTSAGREIKNKEEILSLLEALHLPKRLAIIHCPGHQK
AKDPISRGNQMADRVAKQAA
SEQ ID NO: 41 bmx-C-RT-M5.1 + Y63R
TLQLDDEYRLYSPLVKPDQNIQFWLEQFPQAWAETAGVGLAKQVPPQVIQLKASATPVSVRQRPLSKEARE
GIRPHVQRLIQQGILVPVQSPWNTPLLPVRKPGINDYRPVQDLREVNKRVQDIHPTVPNPYNLLCALPPQR
SWYTVLDLKDAFFCLRLAPTSQPLFAFEWRDPGAGRTGQLTWIRLPQGEKNSPTIENEALARDLANFRIQH
PQVTLLQYADDLLLAGATKQDCLEGTKALLLELSDLGYRASAKKAQICRREVTYLGYSLRGGQRWLTEARK
RTVVQIPAPTTAKQVREFLGKAGFCRLFIPGFATLAAPLYPLTKEKGEFSWAPEHQKAFDAIKKALLSAPA
LALPDVTKPFTLYVDERKGVARGVLTQTLGPWRRPVAYLSKKLDPVASGWPVCLKAIAAVAILVKDADKLT
LGQNITVIAPHALENIVRQPPDRWMTNARMTHYQSLLITERVTFAPPAALNPATLLPEETDEPVTHDCHQL
LIEETGVRKDLIDIPLTGEVLTWFTDGSSYVVEGKRMAGAAVVDGTRTIWASSLPEGTSAQKAELMALTQA
IRLAEGKSINIYTDSRYAFATAHVAGAIYKQRGWITSAGREIKNKEEILSLLEALHLPKRLAIIHCPGHQK
AKDPISRGNQMADRVAKQAA
SEQ ID NO: 42 bmx-C-RT-M5.1 + C408R
TLQLDDEYRLYSPLVKPDQNIQFWLEQFPQAWAETAGVGLAKQVPPQVIQLKASATPVSVRQYPLSKEARE
GIRPHVQRLIQQGILVPVQSPWNTPLLPVRKPGINDYRPVQDLREVNKRVQDIHPTVPNPYNLLCALPPQR
SWYTVLDLKDAFFCLRLAPTSQPLFAFEWRDPGAGRTGQLTWTRIPQGEKNSPTIENEALARDLANFRIQH
PQVTLLQYADDLLLAGATKQDCLEGTKALLLELSDLGYRASAKKAQICRREVTYLGYSLRGGQRWLTEARK
RTVVQIPAPTTAKQVREFLGKAGFCRLFIPGFATLAAPLYPLTKEKGEFSWAPEHQKAFDAIKKALLSAPA
LALPDVTKPFTLYVDERKGVARGVLTQTLGPWRRPVAYLSKKLDPVASGWPVRIKAIAAVAILVKDADKLT
LGQNITVIAPHALENIVRQPPDRWMTNARMTHYQSLLITERVTFAPPAALNPATLLPEETDEPVTHDCHQL
LIEETGVRKDLTDIPLTGEVLTWFTDGSSYVVEGKRMAGAAVVDGTRTIWASSLPEGTSAQKAELMALTQA
IRLAEGKSINIYTDSRYAFATAHVHGAIYKQRGWLTSAGREIKNKEEILSLLEALHLPKRLAIIHCPGHQK
AKDPISRGNQMADRVAKQAA
SEQ ID NO: 43bmx-C-RT-M7
TLQLDDEYRLYSPLVKPDQNIQFWLEQFPQAWAETAGVGLAKQVPPQVIQLKASATPVSVRQRPLSKEARE
GIRPHVQRLIQQGILVPVQSPWNTPLLPVRKPGTNDYRPVQDLREVNKRVQDIHPTVPNPYNLLCALPPQR
SWYTVLDLKDAFFCLRLHPTSQPLFAFEWRDPGAGRTGQLTWTRLPQGFKNSPTIFNEALHRDLANFRIQH
PQVTLLQYADDLLLAGATKQDCLEGTKALLLELSDLGYRASAKKAQICRREVTYLGYSLRGGQRWLTEARK
RTVVQIPAPTTAKQVREFLGKAGFCRLFIPGFATLAAPLYPLTKEKGEFSWAPEHQKAFDAIKKALLSAPA
LALPDVTKPFTLYVDERKGVARGVLTQTLGPWRRPVAYLSKKLDPVASGWPVRLKAIAAVAILVKDADKLT
LGQNITVIAPHALENIVRQPPDRWMTNARMTHYQSLLLTERVTFAPPAALNPATLLPEETDEPVTHDCHQL
LIEETGVRKDLTDIPLTGEVLTWFTDGSSYVVEGKRMAGAAVVDGTRTIWASSIPEGTSAQKAELMALTQA
LRLAEGKSINIYTDSRYAFATAHVHGAIYKQRGWLTSAGREIKNKEEILSLLEALHLPKRLAIIHCPGHQK
AKDPISRGNQMADRVAKQAA
SEQ ID NO: 44 bmx-C-RT-M7Δ
TLQLDDEYRLYSPLVKPDQNIQFWLEQFPQAWAETAGVGLAKQVPPQVIQLKASATPVSVRQRPLSKEARE
GIRPHVQRLIQQGILVPVQSPWNTPLLPVRKPGTNDYRPVQDLREVNKRVQDIAPTVPNPYNLLCALPPQR
SWYTVLDLKDAFFCLRLHPTSQPLFAFEWRDPGAGRTGQLTWTRLPQGFKNSPTIENEALHRDLANFRIQH
PQVILLQYADDLLLAGATKQDCLEGTKALLLELSDLGYRASAKKAQICRREVTYLGYSLRGGQRWLTEARK
RTVVQIPAPTTAKQVREFLGKAGFCRLFIPGFATLAAPLYPLTKEKGEFSWAPEHQKAFDAIKKALLSAPA
LALPDVTKPFTLYVDERKGVARGVLTQTLGPWRRPVAYLSKKLDPVASGWPVRLKAIAAVAILVKDADKLT
LGQNITVIAPHALENIVRQPPDRWMTNARMTHYQSLLITERVTFAPPAALNPATLLPEETDEPVTHDCHQL
LIEETGVRKDLTDIPL
SEQ ID NO: 45 bmx-pPE-C-M5.1
MKRTADGSEFESPKKKRKVDKKYSIGLDIGTNSVGWAVITDEYKVPSKKEKVLGNTDRHSIKKNLIGALLF
DSGETAEATRLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKERGHFLIEGDLNPDNSDVDKLFIQLVQTYNQL
FEENPINASGVDAKAILSARLSKSRRLENLIAQLPGEKKNGLFGNLIALSIGLTPNEKSNFDLAEDAKLQL
SKDTYDDDLDNLLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKINREDLERKQRTFDNG
SIPHQIHLGELHAILRRQEDFYPFLKDNREKIEKILTFRIPYYVGPLARGNSRFAWMIRKSEETITPWNFE
EVVDKGASAQSFIERMINEDKNLPNEKVLPKHSLLYEYFTVYNELTKVKYVTEGMRKPAFLSGEQKKAIVD
LLFKTNRKVTVKQLKEDYFKKIECFDSVEISGVEDRENASLGTYHDLLKIIKDKDELDNEENEDILEDIVL
TLTLFEDREMIEERLKTYAHLEDDKVMKQLKRRRYTGWGRISRKLINGIRDKQSGKTILDELKSDGFANRN
FMQLIHDDSLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQLLNAKLITQRKEDNLTKAER
GGLSELDKAGFIKRQLVETRQITKHVAQILDSRMNTKYDENDKLIREVKVITLKSKLVSDERKDFQFYKVR
EINNYHHAHDAYLNAVVGTALIKKYPKLESEEVYGDYKVYDVRKMIAKSEQEIGKATAKYFFYSNIMNFFK
TEITLANGEIRKRPLIEINGETGEIVWDKGRDFATVRKVLSMPQVNIVKKTEVQTGGFSKESILPKRNSDK
LIARKKDWDPKKYGGEDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQLEVE
QHKHYLDEIIEQISEFSKRVILADANLDKVLSAYNKARDKPIREQAENIIHLFTLTNLGAPAAFKYFDTTI
DRKRYTSTKEVLDATLIHQSITGLYETRIDLSQLGGDSGGSSGGSSGSETPGTSESATPESSGGSSGGSST
LQLDDEYRLYSPLVKPDQNIQFWLEQFPQAWAETAGVGLAKQVPPQVIQLKASATPVSVRQYPLSKEAREG
IRPHVQRLIQQGILVPVQSPWNTPLLPVRKPGINDYRPVQDLREVNKRVQDIHPTVPNPYNLLCALPPQRS
WYTVLDLKDAFFCLRLHPTSQPLFAFEWRDPGAGRTGQLTWTRLPQGFKNSPTIENEALHRDLANFRIQHP
QVTLEQYADDLLLAGATKQDCLEGTKALLLELSDLGYRASAKKAQICRREVTYLGYSLRGGQRWLTEARKR
TVVQIPAPTTAKQVREFLGKAGFCRLFIPGFATLAAPLYPLTKEKGEFSWAPEHQKAFDAIKKALLSAPAL
ALPDVTKPFTLYVDERKGVARGVLTQTLGPWRRPVAYLSKKLDPVASGWPVCLKAIAAVAILVKDADKLTL
GQNITVIAPHALENIVRQPPDRWMTNARMTHYQSLLLTERVTFAPPAALNPATLLPEETDEPVTHDCHQLL
IEETGVRKDLTDIPLTGEVLTWFTDGSSYVVEGKRMAGAAVVDGTRTIWASSLPEGTSAQKAELMALTQAL
RLAEGKSINIYTDSRYAFATAHVEGAIYKQRGWLTSAGREIKNKEEILSLLEALHLPKRLAIIHCPGHQKA
KDPISRGNQMADRVAKQAA.
SEQ ID NO: 46 bmx-pPE-C-M5.1 + Y63R
MKRTADGSEFESPKKKRKVDKKYSIGLDIGINSVGWAVITDEYKVPSKKFKVLGNTDRHSIKKNLIGALLF
DSGETAEATRLKRTARRRYTRRKNRICYLQEIFSNEMAKVDDSFFHRLEESFLVEEDKKHERHPIFGNIVD
EVAYHEKYPTIYHLRKKLVDSTDKADERLIYLALAHMIKERGHFLIEGDLNPDNSDVDKLFIQLVQTYNQL
FEENPINASGVDAKAILSARLSKSRRLENLIAQLPGEKKNGLFGNLIALSLGLTPNEKSNFDLAEDAKLQL
SKDTYDDDLDNLLAQIGDQYADLFLAAKNLSDAILLSDILRVNTEITKAPLSASMIKRYDEHHQDLTELKA
LVRQQLPEKYKEIFFDQSKNGYAGYIDGGASQEEFYKFIKPILEKMDGTEELLVKINREDLLRKQRTFDNG
SIPHQIHLGELHAILRRQEDFYPELKDNREKIEKILTFRIPYYVGPLARGNSRFAWMIRKSEETITPWNFE
EVVDKGASAQSFIERMTNFDKNLPNEKVLPKHSLLYEYETVYNELIKVKYVTEGMRKPAFLSGEQKKAIVD
LLFKTNRKVTVKQLKEDYFKKIECFDSVEISGVEDRFNASLGTYHDLLKIIKDKDELDNEENEDILEDIVL
TITLFEDREMIEERLKTYAHLEDDKVMKQLKRRRYTGWGRLSRKLINGIRDKQSGKTILDELKSDGEANRN
FMQLIKDDSLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQLLNAKLITQRKEDNITKAER
GGLSELDKAGFIKRQLVETRQITKHVAQILDSRMNTKYDENDKLIREVKVITLKSKEVSDERKDFQFYKVR
EINNYHHAHDAYLNAVVGTALIKKYPKLESEFVYGDYKVYDVRKMIAKSEQEIGKATAKYFFYSNIMNFFK
TEITLANGEIRKRPLIETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEKNPIDFLEAKGYKE
VKKDLIIKLPKYSIFELENGRKRMLASAGELQKGNELALPSKYVNFLYLASHYEKLKGSPEDNEQKQLEVE
QHKHYLDEIIEQISEFSKRVILADANLDKVLSAYNKHRDKPIREQAENIIHLFTLTNLGAPAAFKYFDTTI
DRKRYTSTKEVLDATLIHQSITGLYETRIDLSQLGGDSGGSSGGSSGSETPGTSESATPESSGGSSGGSST
LQLDDEYRLYSPLVKPDQNIQFWLEQFPQAWAETAGVGLAKQVPPQVIQLKASATPVSVRQRPLSKEAREG
IRPHVQRLIQQGILVPVQSPWNTPLLPVRKPGINDYRPVQDLREVNKRVQDIHPTVPNPYNLLCALPPQRS
WYTVLDLKDAFFCLRLAPTSQPLEAFEWRDPGAGRTGQLTWTRLPQGEKNSPTIENEALARDLANFRIQHP
QVTLLQYADDLLLAGATKQDCLEGTKALLLELSDLGYRASAKKAQICRREVTYLGYSLRGGQRWLTEARKR
TVVQIPAPTTAKQVREFLGKAGFCRLFIPGFATLAAPLYPLTKEKGEFSWAPEHQKAFDAIKKALLSAPAL
ALPDVTKPFTLYVDERKGVARGVLTQTLGPWRRPVAYLSKKLDPVASGWPVCLKAIAAVAILVKDADKLTL
GQNITVIAPHALENIVRQPPDRWMTNARMTHYQSLLLTERVTFAPPAALNPATLLPEETDEPVTHDCHQLL
IEETGVRKDLTDIPLTGEVLTWFTDGSSYVVEGKRMAGAAVVDGTRTIWASSLPEGTSAQKAELMALTQAL
RLAEGKSINIYTDSRYAFATAHVHGAIYKQRGWITSAGREIKNKEEILSLLEALHLPKRLAIIHCPGHQKA
KDPISRGNQMADRVAKQAA.
SEQ ID NO: 47 bmx-pPE-C-M5.1 + C408R
MKRTADGSEFESPKKKRKVDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESFIVEEDKKHERHPIFGNIVD
EVAYHEKYPTIYHLRKKLVDSTDKADERLIYLALAHMIKERGHFLIEGDLNPDNSDVDKLFIQLVQTYNQL
FEENPINASGVDAKAILSARLSKSRRLENLIAQLPGEKKNGLEGNLIALSLGLTPNFKSNFDLAEDAKLQL
SKDTYDDDLDNLLAQIGDQYADLFLAAKNLSDAILLSDILRVNTEITKAPLSASMIKRYDEHHQDLTELKA
LVRQQLPEKYKEIFFDQSKNGYAGYIDGGASQEEFYKFIKPILEKMDGTEELLVKINREDLLRKQRTFDNG
SIPHQIHLGELHAILRRQEDFYPFLKDNREKIEKILTFRIPYYVGPLARGNSRFAWMIRKSEETITPWNFE
EVVDKGASAQSFIERMINEDKNLPNEKVLPKHSLLYEYETVYNELTKVKYVTEGMRKPAFLSGEQKKAIVD
LLFKTNRKVTVKQLKEDYFKKIECFDSVEISGVEDRENASLGTYHDLLKIIKDKDELDNEENEDILEDIVL
TITLFEDREMIEERLKTYAHLEDDKVMKQLKRRRYTGWGRLSRKLINGIRDKQSGKTILDELKSDGFANRN
FMQLIHDDSLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQELDINRLS
DYDVDAIVPQSFLKDDSIDNKVUTRSDKNRGKSDNVPSEEVVKKMKNYWRQLINAKLITQRKEDNITKAER
GGLSELDKAGFIKRQLVETRQITKHVAQILDSRMNTKYDENDKLIREVKVITLKSKLVSDERKDFQFYKVR
EINNYHHAHDAYLNAVVGTALIKKYPKLESEFVYGDYKVYDVRKMIAKSEQEIGKATAKYFFYSNIMNEFK
TEITLANGEIRKRPLIEINGETGEIVWDKGRDFATVRKVLSMPQVNIVKKTEVQTGGFSKESILPKRNSDK
LIARKKDWDPKKYGGEDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQLEVE
QHKHYLDEIIEQISEFSKRVILADANLDKVLSAYNKHRDKPIREQAENIIHIFTLINLGAPAAFKYFDTTI
DRKRYTSTKEVLDATLIHQSITGLYETRIDLSQLGGDSGGSSGGSSGSETPGTSESATPESSGGSSGGSST
LQLDDEYRLYSPLVKPDQNIQFWLEQFPQAWAETAGVGLAKQVPPQVIQLKASATPVSVRQYPLSKEAREG
IRPHVQRLIQQGILVPVQSPWNTPLLPVRKPGTNDYRPVQDLREVNKRVQDIHPTVPNPYNLLCALPPQRS
WYTVLDLKDAFFCLRLHPTSQPLFAFEWRDPGAGRTGQLTWTRLPQGFKNSPTIFNEALHRDLANFRIQHP
QVTLLQYADDLLLAGATKQDCLEGTKALLLELSDLGYRASAKKAQICRREVTYLGYSLRGGQRWLTEARKR
TVVQIPAPTTAKQVREFLGKAGFCRLFIPGFATLAAPLYPLTKEKGEFSWAPEHQKAFDAIKKALLSAPAL
ALPDVTKPFTLYVDERKGVARGVLTQTLGPWRRPVAYLSKKLDPVASGWPVRLKAIAAVAILVKDADKLIL
GQNITVIAPHALENIVRQPPDRWMTNARMTHYQSLLLTERVTFAPPAALNPATLLPEETDEPVTHDCHQLL
IEETGVRKDLTDIPLTGEVLTWFTDGSSYVVEGKRMAGAAVVDGTRTIWASSLPEGTSAQKAELMALTQAL
RLAEGKSINIYTDSRYAFATAHVHGAIYKQRGWLTSAGREIKNKEEILSLLEALHLPKRLAIIHCPGHQKA
KDPISRGNQMADRVAKQAA
SEQ ID NO: 48 bmx-pPE-C-M7
MKRTADGSEFESPKKKRKVDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKERGHFLIEGDLNPDNSDVDKLFIQLVQTYNQL
FEENPINASGVDAKAILSARLSKSRRLENLIAQLPGEKKNGLFGNLIALSLGLTPNEKSNFDLAEDAKLQL
SKDTYDDDLDNLLAQIGDQYADLFLAAKNISDAILLSDILRVNTEITKAPISASMIKRYDEHHQDLTLLKA
LVRQQLPEKYKEIFFDQSKNGYAGYIDGGASQEEFYKFIKPILEKMDGTEELLVKINREDLLRKQRTFDNG
SIPHQIHLGELHAILRRQEDFYPELKDNREKIEKILTFRIPYYVGPLARGNSRFAWMIRKSEETITPWNFE
EVVDKGASAQSFIERMINFDKNIPNEKVLPKHSLLYEYFTVYNELTKVKYVTEGMRKPAFLSGEQKKAIVD
LLFKTNRKVTVKQLKEDYFKKIECFDSVEISGVEDRENASLGTYHDLLKIIKDKDELDNEENEDILEDIVL
TLTLFEDREMIEERLKTYAHLEDDKVMKQLKRRRYTGWGRLSRKLINGIRDKQSGKTILDFLKSDGFANRN
FMQLIKDDSLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQLLNAKLITQRKEDNITKAER
GGLSELDKAGFIKRQLVETRQITKHVAQILDSRMNTKYDENDKLIREVKVITLKSKLVSDERKDFQFYKVR
EINNYHHAHDAYINAVVGTALIKKYPKLESEFVYGDYKVYDVRKMIAKSEQEIGKATAKYFFYSNIMNFFK
TEITLANGEIRKRPLIEINGETGEIVWDKGRDEATVRKVLSMPQVNIVKKTEVQTGGFSKESILPKRNSDK
LIARKKDWDPKKYGGEDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLINLGAPAAFKYFDTTI
DRKRYTSTKEVLDATLIKQSITGLYETRIDLSQLGGDSGGSSGGSSGSETPGTSESATPESSGGSSGGSST
LQLDDEYRLYSPLVKPDQNIQFWLEQFPQAWAETAGVGLAKQVPPQVIQLKASATPVSVRQRPLSKEAREG
IRPHVQRLIQQGILVPVQSPWNTPLLPVRKPGTNDYRPVQDLREVNKRVQDIHPTVPNPYNLLCALPPQRS
WYTVLDLKDAFFCLRLHPTSQPLFAFEWRDPGAGRTGQLTWTRLPQGFKNSPTIENEALHRDLANFRIQHP
QVTLLQYADDLLLAGATKQDCLEGTKALLLELSDLGYRASAKKAQICRREVTYLGYSLRGGQRWLTEARKR
TVVQIPAPTTAKQVREFLGKAGFCRLFIPGFATLAAPLYPLTKEKGEFSWAPEHQKAFDAIKKALLSAPAL
ALPDVTKPETLYVDERKGVARGVLTQTLGPWRRPVAYLSKKLDPVASGWPVRLKAIAAVAILVKDADKLTL
GQNITVIAPHALENIVRQPPDRWMTNARMTHYQSLLITERVTFAPPAALNPATLLPEETDEPVTHDCHQLL
IEETGVRKDETDIPLTGEVLTWFTDGSSYVVEGKRMAGAAVVDGTRTIWASSLPEGTSAQKAELMALTQAL
RLAEGKSINIYTDSRYAFATAHVAGAIYKQRGWITSAGREIKNKEEILSLLEALHLPKRLAIIHCPGHQKA
KDPISRGNQMADRVAKQAA
SEQ ID NO: 49 bmx-epPE-C-M7Δ
MKRTADGSEFESPKKKRKVDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESELVEEDKKHERHPIFGNIVD
EVAYHEKYPTIYHLRKKLVDSTDKADLRLIYLALAHMIKERGHFLIEGDLNPDNSDVDKLFIQLVQTYNQL
FEENPINASGVDAKAILSARLSKSRRLENLIAQLPGEKKNGLFGNLIALSLGLTPNEKSNFDLAEDAKLQL
SKDTYDDDLDNLLAQIGDQYADLFLAAKNLSDAILLSDILRVNTEITKAPLSASMIKRYDEHHQDLTELKA
LVRQQLPEKYKEIFFDQSKNGYAGYIDGGASQEEFYKFIKPILEKMDGTEELLVKINREDLLRKQRTFDNG
SIPHQIHLGELHAILRRQEDFYPFLKDNREKIEKILTFRIPYYVGPLARGNSRFAWMIRKSEETITPWNFE
EVVDKGASAQSFIERMTNEDKNLPNEKVLPKASLLYEYFTVYNELIKVKYVTEGMRKPAFLSGEQKKAIVD
LLEKTNRKVTVKQLKEDYFKKIECFDSVEISGVEDRFNASLGTYHDLLKIIKDKDELDNEENEDILEDIVL
TLTLFEDREMIEERLKTYAHLEDDKVMKQLKRRRYTGWGRLSRKLINGIRDKQSGKTILDELKSDGFANRN
FMQLIKDDSLTEKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQELDINRIS
DYDVDAIVPQSFLKDDSIDNKVLTRSDKNRGKSDNVPSEEVVKKMKNYWRQLLNAKLITQRKEDNLTKAER
GGLSELDKAGFIKRQLVETRQITKHVAQILDSRMNTKYDENDKLIREVKVITLKSKEVSDERKDFQFYKVR
EINNYHHAHDAYLNAVVGTALIKKYPKLESEFVYGDYKVYDVRKMIAKSEQEIGKATAKYFFYSNIMNFFK
TEITLANGEIRKRPLIEINGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEKNPIDELEAKGYKE
VKKDLIIKLPKYSLFELENGRKRMLASAGELQKGNELALPSKYVNFLYLASHYEKLKGSPEDNEQKQLEVE
QHKHYLDEIIEQISEFSKRVILADANLDKVLSAYNKHRDKPIREQAENIIHLFTLINLGAPAAFKYFDTTI
DRKRYTSTKEVLDATLIHQSITGLYETRIDLSQLGGDSGGSSGGSSGSETPGTSESATPESSGGSSGGSST
LQLDDEYRLYSPLVKPDQNIQFWLEQFPQAWAETAGVGLAKQVPPQVIQLKASATPVSVRQRPLSKEAREG
IRPHVQRLIQQGILVPVQSPWNTPLLPVRKPGINDYRPVQDLREVNKRVQDIHPTVPNPYNLLCALPPQRS
WYTVLDLKDAFFCLRLHPTSQPLFAFEWRDPGAGRTGQLTWTRLPQGFKNSPTIENEALARDLANFRIQHP
QVTLLQYADDLLLAGATKQDCLEGTKALLLELSDLGYRASAKKAQICRREVTYLGYSLRGGQRWLTEARKR
TVVQIPAPTTAKQVREFLGKAGFCRLFIPGFATLAAPLYPLTKEKGEFSWAPEHQKAFDAIKKALLSAPAL
ALPDVTKPFTLYVDERKGVARGVLTQTLGPWRRPVAYLSKKLDPVASGWPVRLKAIAAVAILVKDADKUTL
GQNITVIAPHALENIVRQPPDRWMTNARMTHYQSLLLTERVTFAPPAALNPATLLPEETDEPVTHDCHQLL
IEETGVRKDLTDIPL
SEQ ID NO: 50 Csy 4
GTTCACTGCCGTATAGGCAG
SEQ ID NO: 51 tCsy4
CTGCCGTATAGGCAG

1. A prime editing system for targeted modification of a genomic DNA sequence in an organism, comprising:
i) (a) a CRISPR nuclease such as a CRISPR nickase and/or an expression construct comprising a nucleotide sequence encoding said CRISPR nuclease such as a CRISPR nickase, and a PERV reverse transcriptase or a functional variant thereof and/or an expression construct comprising a nucleotide sequence encoding said PERV reverse transcriptase or functional variant thereof; or
(b) a prime editing fusion protein and/or an expression construct comprising a nucleotide sequence encoding said prime editing fusion protein, wherein said prime editing fusion protein comprises a CRISPR nuclease such as a CRISPR nickase and a PERV reverse transcriptase or functional variant thereof; and/or
ii) at least one pegRNA and/or an expression construct comprising a nucleotide sequence encoding said at least one pegRNA,
wherein said at least one pegRNA comprises, in the 5′ to 3′ direction, a guide sequence, a scaffold sequence, a reverse transcription template (RTT) sequence, and a primer binding site (PBS) sequence.

2. The prime editing system of claim 1, wherein said PERV reverse transcriptase is derived from type A, B, or C PERV, or a recombinant from different PERV types, such as an A/C-type PERV recombinant.

3. The prime editing system of claim 1 or 2, wherein said PERV is isolated from Bama Xiang pigs.

4. The prime editing system of any one of claims 1-3, wherein said PERV reverse transcriptase or functional variant thereof comprises an amino acid sequence of any one of SEQ ID NOs: 1-5 or an amino acid sequence with having at least 75%, at least 80%, at least 90%, at least 95%, at least 96%, at least 97%, at least 98%, or at least 99% sequence identity to any one of SEQ ID NOs: 1-5.

5. The prime editing system of any one of claims 1-4, wherein the functional variant of said PERV reverse transcriptase lacks an RNase H domain.

6. The prime editing system of any one of claims 1-5, wherein the functional variant of said PERV reverse transcriptase comprises one or more amino acid substitutions at positions selected from 63, 199, 222, 305, 312, 329, 330, 331, 332, 408, or 602.

7. The prime editing system of claim 6, wherein the functional variant of said PERV reverse transcriptase comprises amino acid substitutions at positions 199, 305, and 312.

8. The prime editing system of claim 7, wherein the functional variant of said PERV reverse transcriptase comprises amino acid substitutions D199N, T305K, and W312F.

9. The prime editing system of claim 6, wherein the functional variant of said PERV reverse transcriptase comprises amino acid substitutions at positions 199, 305, 312, and 602.

10. The prime editing system of claim 9, wherein the functional variant of said PERV reverse transcriptase comprises amino acid substitutions D199N, T305K, W312F, and L602W.

11. The prime editing system of claim 6, wherein the functional variant of said PERV reverse transcriptase comprises amino acid substitutions at positions 199, 305, 312, 329, 330, 331, and 332.

12. The prime editing system of claim 11, wherein the functional variant of said PERV reverse transcriptase comprises amino acid substitutions D199N, T305K, W312F, E329P, K330G, G331T, and E332C.

13. The prime editing system of claim 6, wherein the functional variant of said PERV reverse transcriptase comprises amino acid substitutions at positions 199, 305, 312, 329, 330, 331, 332, and 602.

14. The prime editing system of claim 13, wherein the functional variant of said PERV reverse transcriptase comprises amino acid substitutions D199N, T305K, W312F, E329P, K330G, G331T, E332C, and L602W.

15. The prime editing system of claim 6, wherein the functional variant of said PERV reverse transcriptase comprises amino acid substitutions at positions 199, 222, 305, 312, and 602.

16. The prime editing system of claim 15, wherein the functional variant of said PERV reverse transcriptase comprises amino acid substitutions D199N, V222A, T305K, W312F, and L602W.

17. The prime editing system of claim 6, wherein the functional variant of said PERV reverse transcriptase comprises amino acid substitutions at positions 63, 199, 222, 305, 312, and 602.

18. The prime editing system of claim 17, wherein the functional variant of said PERV reverse transcriptase comprises amino acid substitutions Y63R, D199N, V222A, T305K, W312F, and L602W.

19. The prime editing system of claim 6, wherein the functional variant of said PERV reverse transcriptase comprises amino acid substitutions at positions 199, 222, 305, 312, 408, and 602.

20. The prime editing system of claim 19, wherein the functional variant of said PERV reverse transcriptase comprises amino acid substitutions D199N, V222A, T305K, W312F, C408R, and L602W.

21. The prime editing system of claim 6, wherein the functional variant of said PERV reverse transcriptase comprises amino acid substitutions at positions 63, 199, 222, 305, 312, 408, and 602.

22. The prime editing system of claim 13, wherein the functional variant of said PERV reverse transcriptase comprises amino acid substitutions Y63R, D199N, V222A, T305K, W312F, C408R, and L602W.

23. The prime editing system of any one of claims 1-4, wherein the functional variant of said PERV reverse transcriptase comprises an amino acid sequence of any one of SEQ ID NOs: 6-17 or 40-44.

24. The prime editing system of any one of claims 1-23, wherein in said fusion protein, said reverse transcriptase or functional variant thereof is fused directly or via a linker to a nucleocapsid protein (NC) at the N-terminus or C-terminus.

25. The prime editing system of claim 24, wherein said nucleocapsid protein (NC) is derived from PERV, preferably, said nucleocapsid protein and said reverse transcriptase are derived from the same type of PERV.

26. The prime editing system of claim 24 or 25, wherein said nucleocapsid protein comprises an amino acid sequence as shown in any one of SEQ ID NOs: 19 or 36-39.

27. The prime editing system of any one of claims 24-26, wherein said PERV reverse transcriptase or its functional variant is fused directly or via a linker to a nucleocapsid protein (NC) at the N-terminus.

28. The prime editing system of any one of claims 1-27, wherein in said prime editing fusion protein, said CRISPR nuclease such as a CRISPR nickase and said PERV reverse transcriptase or functional variant thereof are connected via a linker.

29. The prime editing system of any one of claims 1-28, wherein said CRISPR nickase is a Cas9 nickase, for example, said Cas9 nickase comprises an amino acid sequence as shown in SEQ ID NO: 18.

30. The prime editing system of any one of claims 1-29, wherein in said fusion protein, said CRISPR nuclease such as a CRISPR nickase is located at the N-terminus of said reverse transcriptase or functional variant thereof.

31. The prime editing system of any one of claims 1-30, wherein said fusion protein further comprises a nuclear localization sequence (NLS).

32. The prime editing system of any one of claims 1-31, wherein said fusion protein further comprises a selectable marker protein connected directly or via a self-cleaving peptide.

33. The prime editing system of claim 32, wherein said self-cleaving peptide is a 2A peptide, and said selectable marker protein is a Puro-R protein.

34. The prime editing system of claim 1, wherein said fusion protein comprises an amino acid sequence as shown in any one of SEQ ID NOs: 22-34 or SEQ ID NOs: 45-49.

35. The prime editing system of any one of claims 1-34, wherein said at least one pegRNA is capable of forming a complex with said fusion protein and targeting said fusion protein to a target sequence in the genome, resulting in a nick within said target sequence.

36. The prime editing system of any one of claims 1-35, wherein the guide sequence in said at least one pegRNA is designed to have sufficient sequence identity, preferably 100% identity, to the target sequence to enable sequence-specific targeting through base pairing with the complementary strand of the target sequence.

37. The prime editing system of any one of claims 1-36, wherein said scaffold sequence is as shown in SEQ ID NO: 21.

38. The prime editing system of any one of claims 1-37, wherein said primer binding sequence is designed to be complementary to at least a portion of said target sequence, preferably, said primer binding sequence is complementary to at least a portion of the 3′ overhang single strand generated by the nick, particularly complementary to the nucleotide sequence at the 3′ end of said 3′ overhang single strand.

39. The prime editing system of any one of claims 1-38, wherein said RT template sequence is designed to correspond to the sequence downstream of the nick and comprises desired modifications, including substitution, deletion, and/or addition of one or more nucleotides.

40. The prime editing system of any one of claims 1-39, further comprising a nick gRNA and/or an expression construct comprising a nucleotide sequence encoding said nick gRNA, wherein said nick gRNA comprises a guide sequence and a scaffold sequence, said guide sequence being designed to have sufficient sequence identity to a target sequence in the genome to enable targeting of said fusion protein to said target sequence and generation of a nick within said target sequence, wherein the target sequence of said nick gRNA and the target sequence of said pegRNA are located on opposite strands of the genomic DNA, and the nick induced by said nick gRNA and the nick induced by said pegRNA are spaced approximately 1 to approximately 300 nucleotides apart.

41. The prime editing system of any one of claims 1-40, comprising at least one pair of pegRNAs and/or an expression construct comprising a nucleotide sequence encoding said at least one pair of pegRNAs.

42. The prime editing system of claim 41, wherein the two pegRNAs in said pegRNA pair are designed to target different target sequences on the same strand of genomic DNA, or the two pegRNAs in said pegRNA pair are designed to target sequences on different strands of genomic DNA.

43. The prime editing system of claim 41 or 42, wherein the two pegRNAs in said pegRNA pair are designed to introduce the same desired modification.

44. The prime editing system of any one of claims 1-43, wherein said pegRNA comprises a truncated Csy4 recognition sequence as shown in SEQ ID NO: 51 at the 3′ end.

45. A method for generating a genetically modified cell, comprising introducing the prime editing system of any one of claims 1-44 into at least one said cell, thereby modifying the genomic sequence of said at least one cell.

46. The method of claim 45, wherein said prime editing system is introduced into said cell in the presence of one or more small molecule compounds selected from RS-1, Nocodazole, and Trichostatin A (TSA) (preferably RS-1 and Nocodazole); or
after introducing said prime editing system into said cell, treating said cell with one or more small molecule compounds selected from RS-1, Nocodazole, and Trichostatin A (TSA) (preferably RS-1 and Nocodazole), for example, culturing said cell in a medium comprising said one or more small molecule compounds (preferably RS-1 and Nocodazole).

47. The method of claim 45 or 46, wherein said cell is derived from a mammal, such as a human, mouse, rat, monkey, dog, pig, sheep, cow, or cat.
